(19) United States
(12) Patent Application Publication
(10) Pub. No.: US 2024/0116864 A1
(43) Pub. Date: Apr. 11, 2024

(54) XANTHOPHYLL DERIVATIVES

(71) Applicants: ORN ALMARSSON, CAMBRIDGE, MA (US); CARLOS N. SANRAME, LEXINGTON, MA (US); JOHN LUCAS, MAIDENHEAD (GB)

(72) Inventors: ORN ALMARSSON, CAMBRIDGE, MA (US); CARLOS N. SANRAME, LEXINGTON, MA (US); JOHN LUCAS, MAIDENHEAD (GB)

(21) Appl. No.: 18/269,636

(22) PCT Filed: Dec. 22, 2021
(86) PCT No.: PCT/US2021/064918

§ 371 Date: Jun. 26, 2023

Related U.S. Application Data
(60) Provisional application No. 63/130,435, filed on Dec. 24, 2020.

(51) Int. Cl. C07C 403/24; C07D 303/28; C07D 407/06

(52) U.S. Cl. CPC C07C 403/24; C07D 303/28; C07D 407/06; C07C 2601/16

(57) ABSTRACT

The present invention provides for xanthophyll derivatives and compositions comprising the xanthophyll derivatives as well methods of making and using the same. The compositions include pharmaceutical compositions as well as foodstuffs, food additives, food supplements, feedstuffs and feed additives.

### PRIORITY

This application claims priority to U.S. Provisional Application No. 63/130,435, filed on 24 Dec. 2020, which is hereby incorporated by reference in its entirety.

### FIELD OF THE INVENTION

The field of the invention relates to derivatives, including prodrugs, of xanthophylls as well as methods of making and using the same.

### BACKGROUND OF THE INVENTION

Carotenoids are a group of natural pigments produced principally by plants, yeast, and microalgae. All carotenoids share common chemical features, such as a polyisoprenoid structure, a long polyene chain forming the chromophore, and near symmetry around the central double bond. Carotenoids without oxygenated functional groups are called “carotenes”, reflecting their hydrocarbon nature. Oxygenated carotenes are known as “xanthophylls.”

Xanthophylls include astaxanthin formulas (I-VII), zeaxanthin formula (VIII), lutein formula (IX), cryptoxanthin formula (X), antheraxanthin formula (XI), fucoxanthin formula (XII), neoxanthin formulas (XIII and XIV) and violaxanthin formula (XV).

Astaxanthin is a red-orange, non-provitamin A carotenoid and potent antioxidant that is produced naturally, e.g., in the freshwater microalgae Haematococcus pluvialis and the basidiomycetous yeast Xanthophyllomyces dendrorhous (anamorph Phaffia rhodozyma). Animals that feed on the algae, such as salmon, red trout, red sea bream, flamingos, and crustaceans (i.e., shrimp, krill, crab, lobster, and crayfish), subsequently reflect the astaxanthin pigmentation to various degrees. The antioxidant mechanism of astaxanthin include singlet oxygen quenching, direct radical scavenging, and lipid peroxidation chain-breaking.

Astaxanthin has an asymmetric center in the 3 and 3′ position and can therefore exist as a (3S, 3′S) isomer as shown in formula (I); a (3S, 3′R) isomer as shown in formula (II); or a (3R, 3′R) isomer as shown in formula (III). Depending on the source, astaxanthin may be synthesized or isolated as mixed isomers, as a racemate of (3R, 3′R) and (3S, 3′S) with or without the meso-compound II, or as enantiomerically pure astaxanthin. Isomerism around C═C double bonds in the polyene chain yields distinctly different molecular structures that may be isolated as separate compounds (known as Z (“cis”) and E (“trans”), or geometric, isomers). The all-E configuration is an extended, linear molecule (formula (IV)). The presence of any Z in the polyene chain creates a bent-chain molecule (formulas (V-VI).

Absorption of carotenoids, including xanthophylls, from the diet occurs by passive diffusion into the intestinal epithelium, a process that requires fat (van het Hof et al. J Nutr 2000 March; 130(3):503-6; Ribaya-Mercado, Nutrition Reviews 2002 60(4):104-110) and is facilitated by pancreatic phospholipase A2 and lysophosphatidylcholine (Sugawara et al., J Nutr. 2001 November; 131(11):2921-7). Following absorption, carotenoids are incorporated in chylomicrons, transported to the liver via lymph and blood, and partly re-secreted with lipoproteins. Structural differences including geometrical E/Z isomerization cause individual patterns of absorption, plasma transport, and metabolism for carotenoids (Clevidence and Bieri, Methods in Enzymology, 1993, 214:33-46).

Astaxanthin is a strong antioxidant, anti-inflammatory, anti-apoptotic, and immune modulator that has gained growing interest as a multi-target pharmacological agent against various diseases. For example, in cardiovascular and metabolic health, astaxanthin lowers the blood sugar level and improves various parameters of metabolic syndrome (See e.g., K. Uchiyama, Redox. Rep. 2000, 7(5): 290-3; G. Hussein et al., J. Pharm. Sci. 2007, 100(Suppl. 1):176p; M. Ikeuchi et al. Biosci. Biotechnol. Biochem. 2007, 71:893-9; Naito, Y., K. Uchiyama, et al. Biofactors 2004, 20(1): 49-59). In blood hypertension models, astaxanthin increased blood flow and vascular tone (See e.g., H. Yanai et. al. (2008) Integrated Blood Pressure Control 1:1-3). Astaxanthin inhibits the oxidation of low-density lipoproteins and has been proposed for preventing arteriosclerosis (T. Iwamoto et al., J. Atheroscler, Thromb. 2000, 7: 216-222).

In immune health, astaxanthin is an immune system modulator with anti-inflammatory effect and has been proposed for the treatment of autoimmune diseases such as Crohn's disease and inflammatory joint diseases such as arthritis (See e.g., H. Jyonouchi et al. J. Nutr. 1995, 125: 1570-1573; J. Park, et al. Nutr. Metab. 2010, 7: 18-27; Kurashige et al. Physiol. Chem. Phys. Med. 1990, NMR22:27-38; K. Ohgami et al. Investigative Ophthalmology & Visual Sci. 2003, 44(6):2694-2701; and Y. Suzuki et al. Exp. Eye Res. 2006, 82(2): 275-281; Y. Nir, G. Spiller. J Am Coll Nutr, 2002, 21; WO 2019/065674 A1; WO 2019/159868).

In eye health, astaxanthin is used for dry eye, asthenopia, macular degeneration, cataracts, glaucoma diabetic retinopathy, ocular hypertension and ocular ischemia damage of the lens and of the retina by UV radiation (K. Sawaki et al. Journal of Clinical Therapeutics & Medicines, 2002, 18: 73-88; WO 2015/114900; WO 2019/119672; WO 2019/138995;).

In skin health, astaxanthin is used for pruritus, dermatitis, and in delaying skin aging and reducing the development of wrinkles, age spots and freckles (Suganuma K et al., Jichi Medical University Journal. 2012, 35:25-33; K. Tominaga K et al., Acta Biochim Pol. 2012; 59(1):43-7; E. Yamashita, Carotenoid Science. 2006; 10: 91-5; A. Satoh et al. Oyo Yakuri Pharmacometrics, 2011, 80 (1/2): 7-11; WO 2019/111896; WO 2019/111897).

In addition, astaxanthin has shown anti-cancer effects in many cancers, including liver cancer, colon cancer, bladder cancer, oral cancer, leukemia and brain cancer (WO 2020/059745; Zhang L, Mar Drugs. 2015; 13(7):4310-4330; Kowshik J, et al. PLoS One. 2014; 9(10):e109114; Kavitha K, et al. Biochim Biophys Acta. 2013; 1830(10):4433-4444; Li J, Dai W, Xia Y, et al. Mar Drugs. 2015; 13(10):6064-6081)

Other diseases and disorders indicated for astaxanthin include bacterial infections, such as Helicobacter pylori (M. Bennedsen et al., Immunol. Left. 1999, 70: 185-189).

Of particular interest is the use of xanthophylls, especially astaxanthin, in the prevention and treatment of inflammatory, cardiometabolic, neurologic and fibrotic diseases including primary biliary cholangitis (PBC), primary sclerosing cholangitis (PSC), idiopathic pulmonary fibrosis (IPF) and non-alcoholic fatty liver disease (NAFLD). Non-alcoholic fatty liver disease (“NAFLD”) is a condition in which excess fat is stored in the liver (Chalasani, N., et al. Hepatology 2012; 55:2005-2023). NAFLD encompasses a spectrum of chronic liver disease, which includes NAFL, non-alcoholic steatohepatitis (NASH), fibrosis, and cirrhosis (Younossi Z M, et al. Nat Rev Gastroenterol Hepatol. 2018; 15:11-20). Prevalence can be as high as 46% (Cimini, F A., et al. World J Gastroenterol 2017; 23:3407-341). Insulin resistance, a hallmark of physiology underlying NAFLD and may be the cause of fat accumulation in the liver (Neuschwander-Tetri, B A., et al. Hepatology 2003; 37:1202-1219). Two types of NAFLD are simple fatty liver (also called non-alcoholic fatty liver, or “NAFL”) and non-alcoholic steatohepatitis (“NASH”). NAFL is a form of NAFLD in which there is excess fat in the liver but little inflammation or liver cell damage. NASH occurs where there is hepatitis (inflammation of the liver) and liver cell damage in addition to excess fat (Obes Facts. 2016; 9 (2):65-90). Those with NASH are at increased risk of cirrhosis and death from liver disease.

Xanthophylls are effective in preventing and treating NAFLD by exerting antioxidant, lipid-lowering, anti-inflammatory, anti-fibrotic, and insulin-sensitizing properties. Astaxanthin, in particular, is effective in treating the pathogenesis of NAFLD, including NASH, from many aspects. Studies of astaxanthin supplementation on obese mice fed a high fat diet showed that astaxanthin inhibited the increase in body weight and adipose tissue caused by the high fat diet. In addition, astaxanthin also reduced liver weight and liver triglyceride, plasma triglyceride and total cholesterol levels (Ikeuchi M, et al. Biosci Biotechnol Biochem. 2007; 71(4):893-899).

The release of inflammatory factors is crucial in the pathogenesis of NAFLD. Astaxanthin was shown to significantly reduce M1 macrophages and increased M2 macrophages, reduce liver recruitment of CD4⁺ and CD8⁺ and inhibit inflammation in NAFLD. Astaxanthin was more effective than vitamin E at reducing lipid accumulation, improving insulin signal transduction and inhibiting pro-inflammatory signal transduction by inhibiting the activation of Jun N-terminal kinase (JNK)/p38 mitogen-activated protein kinase (MAPK) and NF-κB pathways (Ni Y, Nagashimada M, et al. Sci Rep. 2015; 5(1):17192). Similarly, astaxanthin reduced macrophage infiltration and the expression of macrophage markers in mice, inhibited inflammation and fibrosis in liver and adipose tissue, and enhanced the ability of skeletal muscle to oxidize mitochondrial fatty acids in obese mice (Kim B, et al. J Nutr Biochem. 2017; 43:27-35).

Peroxisome proliferator-activated receptors (PPARs) play an important role in the regulation of inflammation. Activated PPAR-α improves fatty acid transport, metabolism, oxidation and inhibit liver fat accumulation. Conversely, activation and overexpression of PPAR-γ promotes fatty acid storage and lipid accumulation in the liver (Zheng H, et al. Eur J Pharmacol. 2011; 659(2-3):244-251; Cao C Y, et al. Tohoku J Exp Med. 2012; 227(4):253-262. In mice fed a high-fat diet for 8 weeks, astaxanthin reduced liver lipid accumulation induced by a high-fat diet, reduced triglyceride levels in the liver, and decreased the number of inflammatory macrophages and Kupffer cells (Jia Y, et al. J Nutr Biochem. 2016; 28:9-18). These changes were attributed to the regulation of PPARs by astaxanthin. Astaxanthin activates PPAR-α and inhibits the expression of PPAR-γ and the levels of interleukin-6 and tumor necrosis factor-α in the liver, inhibits inflammation and reduces fat synthesis in the liver.

In addition, astaxanthin causes autophagy of hepatocytes by inhibiting the AKT-mTOR pathway and decomposes lipid droplets stored in the liver (Jia Y, et al. J Nutr Biochem. 2016; 28:9-18). Astaxanthin also significantly inhibits the expression of fatty acid synthase and acetyl coenzyme A carboxylase, increases SOD, CAT, GPX activity and glutathione (GSH) in the liver, and significantly reduces lipid peroxidation in the liver (Xu J, et al. Nutrients. 2017; 9(3):271).

In a study using diet-induced obesity (DIO) and nonalcoholic steatohepatitis mouse models, astaxanthin significantly inhibited inflammation and fibrosis in the liver and adipose tissue and enhances the skeletal muscle's capacity for mitochondrial fatty acid oxidation in obese mice (B. Kim, et al. J Nutr Biochem. 2017 May; 43:27-35). US 2016/0287534 A1 discloses the use of astaxanthin as an anti-fibrogenic agent for the reversal of fibrosis and fibrotic diseases including NASH. In U.S. Pat. No. '534, the inventors showed that astaxanthin can reverse the activation of hepatic stellate cells (HSCs) by shifting activated HSCs (aHSCs) to inactivated HSCs (iHCSs). Astaxanthin also inhibits the activation of quiescent HSCs (qH5Cs) to aHSCs.

A disadvantage with the use of free xanthophylls, e.g., astaxanthin, is that they suffer from poor stability, aqueous solubility, absorption and other properties. Previous attempts to create astaxanthin with improved properties, such as solubility and bioavailability can be found in PCT publications WO 2006/039685, WO 2004/011423, WO 2016/037785, WO 2003/066583, WO 2011/095 571, WO 2006/102576, WO 2005/102356, WO 2006/099015 WO 2006/105214, WO 2006/119168, WO 2007/027834, WO 2007/067957, WO 2007/090095, WO 2007/147163, WO 2008/106606, WO 2008/118862, WO 2020/055913, WO 2019/236772, WO 2006/119125, US Patent Applications US2005/0026874, US2005/0009788, US2005/0143475, US2018/0110741, US2005/0228188, US 2017/0081289, US 2018/0055788 and U.S. Pat. No. 10,125,104. However, further improvements leading to enhanced pharmacokinetic and pharmacodynamic profiles are needed. Accordingly, the present invention provides new xanthophyll, particularly astaxanthin, derivatives with improved properties including, but not limited to, greater stability, increased aqueous solubility, increased permeability, improved distribution, increased concentration in a target organ, e.g., liver, increased concentration in mitochondria, and improved bioavailability.

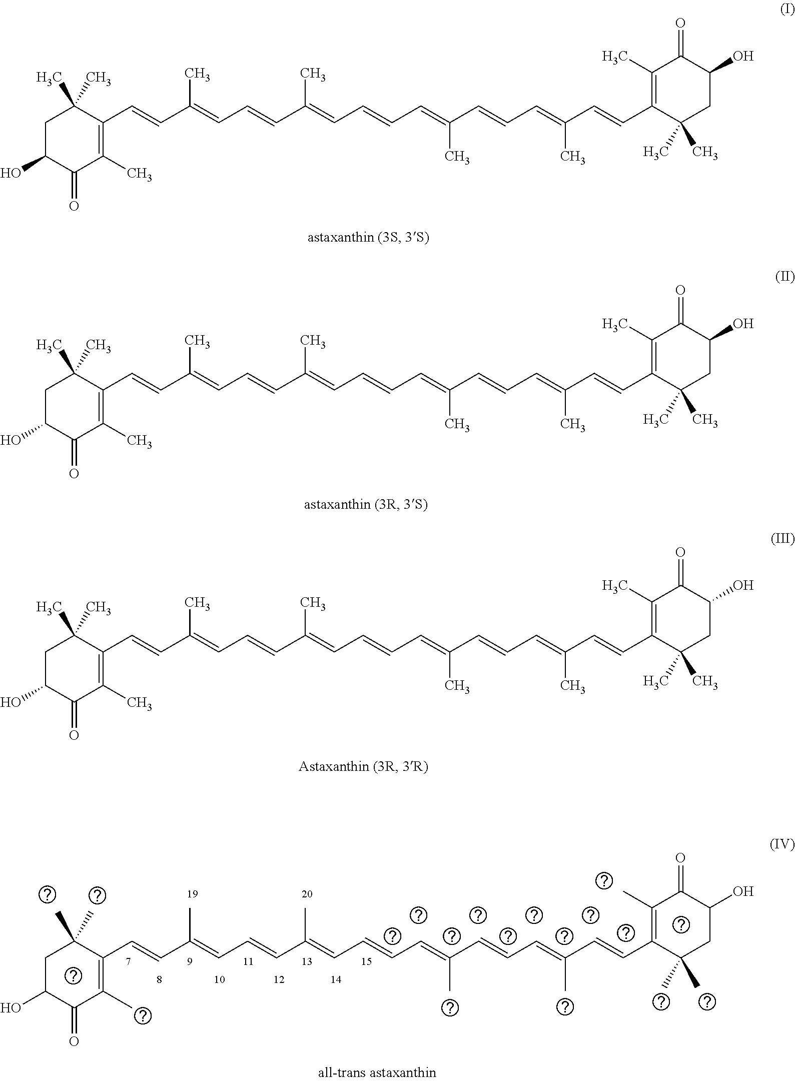

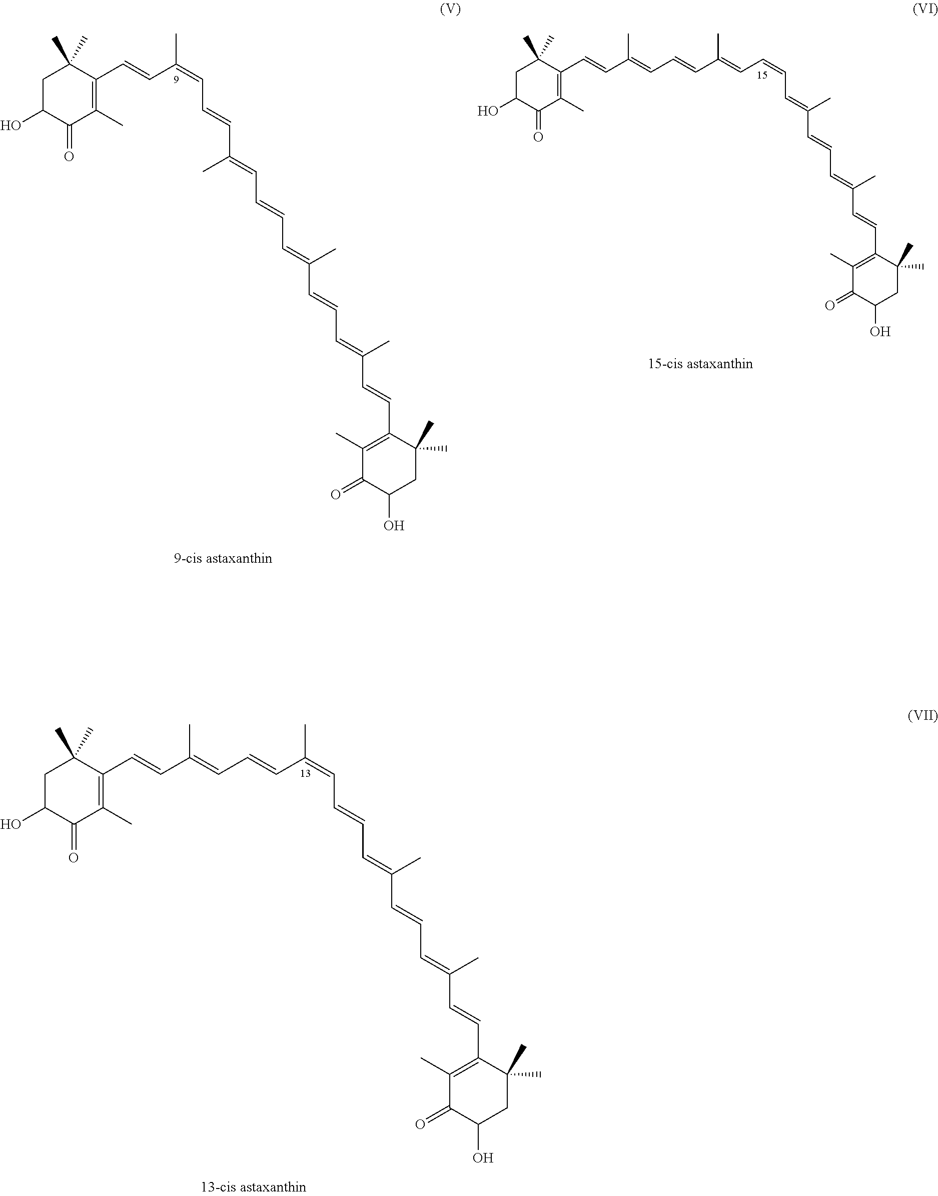

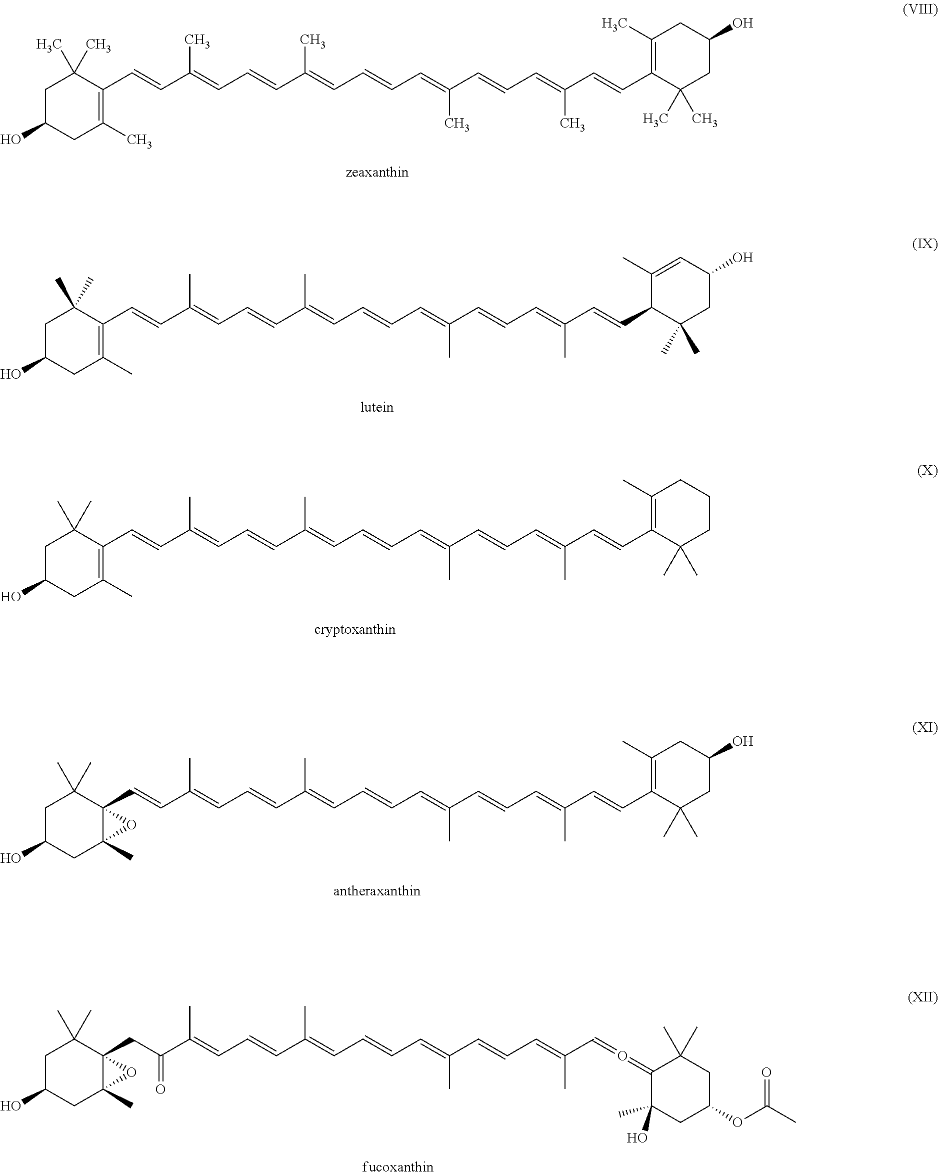

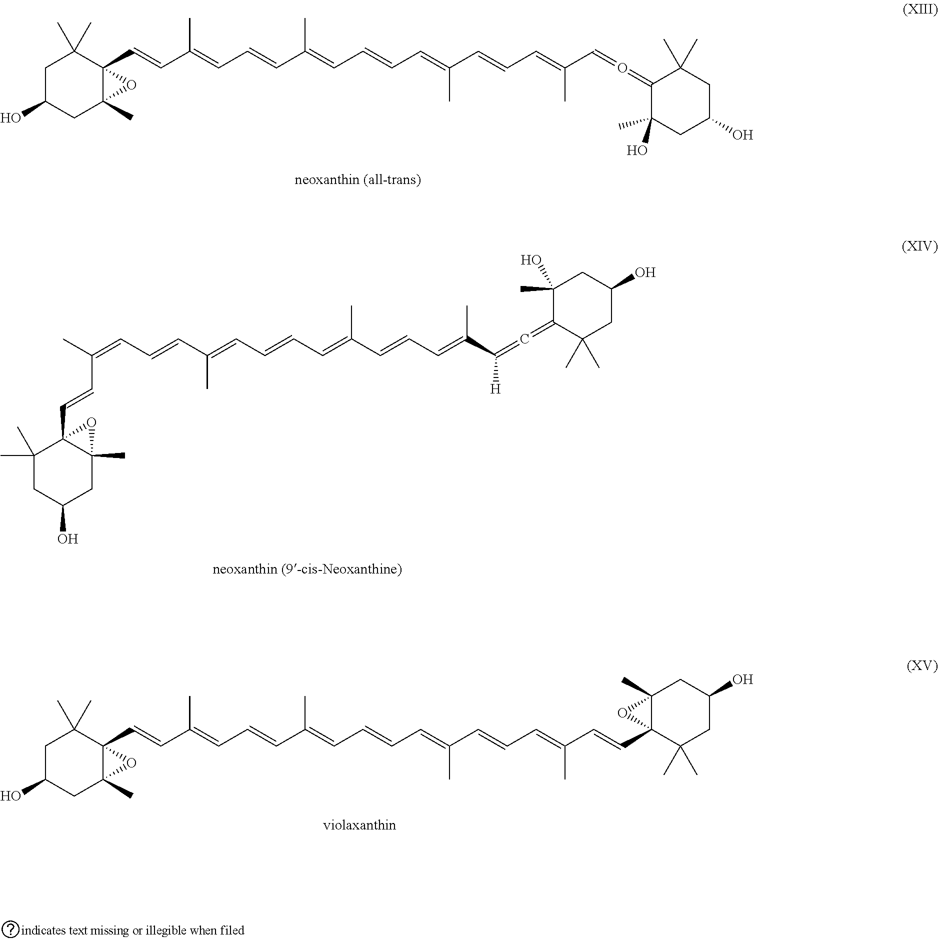

### SUMMARY OF THE INVENTION

The present invention is directed towards new forms of xanthophylls that have improved properties including improved physicochemical characteristics as well as methods for the preparation of compounds.

In one aspect, the invention provides for a compound with a formula selected from any one of formulas (XVI)-(XXX), wherein A1-A31 are independently R1 or —H, and wherein at least one substituent of on any given xanthophyll selected from A1-A31 is R1; and salts, co-crystals, geometric isomers and stereoisomers thereof.

Accordingly, another aspect provides for a method of treating a subject having a disease or disorder that would benefit from the administration of a xanthophyll derivative of the present invention, said method comprising the step of administering to said subject an effective amount of said xanthophyll derivative.

### DETAILED DESCRIPTION OF THE INVENTION

### Definitions

The term “comprising”, as used herein, is synonymous with “including,” “containing,” or “characterized by,” is inclusive or open-ended and does not exclude additional, unrecited elements or method steps, unless the context clearly requires otherwise. For example, a composition of the present invention “comprising an active ingredient” contains one or any number of active ingredients, unless otherwise specified.

The phrases “consists of” or “consisting of” are closed-ended and includes only those features specified. When used in a clause, the phrases “consists of” or “consisting of” limit only the element set forth in that clause.

The phrases “consists essentially of” and “consisting essentially of” are partially open and limited to features that do not materially affect the basic and novel characteristic(s)” of the claimed invention. For example, the phrases include an unrecited level of impurities that do not materially affect the basic and novel characteristic(s), e.g., activity or stability, of a composition of the invention.

As used herein, when a range is set forth as “between” two values, it is understood that the range is inclusive of the end values.

As used herein, the terms “treat”, “treating” or “treatment” means to alleviate, reduce or abrogate one or more symptoms or characteristics of a disease or condition and may be curative, palliative, prophylactic or slow the progression of the disease. The term “therapeutically effective amount” is intended to mean that amount of drug that will elicit a desired biological or pharmacological response, i.e., an amount sufficient to treat said disease.

The term “effective amount” means an amount of active ingredient(s) that will result in a desired effect or result. The term “therapeutically effective amount” means an amount of active ingredient(s) that will elicit a desired biological or pharmacological response, e.g., effective to prevent, alleviate, or ameliorate symptoms of a disease or disorder; slow, halt or reverse an underlying process or progression of a disease or disorder; partially or fully restore cellular function; or prolong the survival of the subject being treated.

The term “subject” means an animal, including mammals, non-human animals, and especially humans. In one embodiment, the subject is a human. In another embodiment, the subject is a human male and in another, the subject is a human female. A “subject” can include an animal, e.g., human subject, for medical or veterinary purposes, such as for the treatment of a disease or condition, but is not limited to such as the compositions of the present invention may be suitable for use in general dietary health and nutrition as foodstuffs, food additives, feedstuffs, feed additives and dietary/food supplements. Suitable animal subjects also include non-human primates and other mammals such as bovines, e.g., cattle, oxen; ovines, e.g., sheep; caprines, e.g., goats; porcines, e.g., pigs, hogs; equines, e.g., horses; felines, e.g., domestic cats; canines, e.g., dogs; lagomorphs, e.g., rabbits, hares; rodents, e.g., mice, rats; birds, particularly fowl for food purposes, e.g., chickens, turkeys, duck, geese; and fish and shellfish including carp, salmon, milkfish, trout, wuchang bream, snakehead, catfish, sea bass, turbot, halibut, sea bream, kingfish, barramundi, grouper and prawns. Further, a “subject” can include a patient afflicted with or suspected of being afflicted with a condition or disease. Thus, the terms “subject” and “patient” may be used interchangeably herein.

The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners.

The term “unit dose” refers to the amount of xanthophyll derivative(s) administered to a subject in a single dose.

The term “xanthophyll derivative” as used herein refers to a xanthophyll that has been covalently modified by removing, substituting, adding, or otherwise altering the chemical structure as disclosed herein. The xanthophyll derivatives of the present invention that are not prodrugs retain biological activity in the modified state. Preferred xanthophyll derivatives are xanthophyll prodrugs, astaxanthin derivatives and astaxanthin prodrugs. Astaxanthin derivatives that are prodrugs include prodrugs that are inactive until metabolized or that retain as least partial biological activity in the prodrug (non-metabolized) form.

The term “xanthophyll prodrug” as used herein refers to a xanthophyll, e.g., astaxanthin, that is covalently modified with a prodrug moiety, i.e., prodrug chemical group. After administration, the prodrug is chemically or enzymatically metabolized in vivo resulting in the formation of the parent xanthophyll compound (e.g., parent 3R, 3R′ all-trans astaxanthin). The xanthophyll prodrugs of the present invention may, in one embodiment, be biologically active or, in another embodiment, be biologically inactive in their prodrug form; wherein the biological activity is the biological activity of the parent xanthophyll. In either case, the prodrug is metabolized in vivo into the parent xanthophyll.

The term “feedstuff” as used herein refers to a product for the nutrition of nonhuman animals, particularly livestock.

The term “feed additive” as used herein refers to a formulation, for example in the form of a powder or a liquid, which can be incorporated into the feedstuff during its manufacture.

The term “foodstuff” as used herein refers to a product for the nutrition of humans, which serves primarily for the intake of nutrients such as fats, proteins and carbohydrates.

The term “food additive” as used herein refers to a formulation, for example in the form of a powder or a liquid, which can be incorporated into the foodstuff during its manufacture.

The term “food supplement” as used herein refers to a formulation which is suitable for oral administration and which is ingested by humans in addition to the traditional food, with the primary aim of administering active substances in order to achieve an improvement of the general diet or the general state of health.

The term “condensed” according to the present invention means that a ring or ring system is attached to another ring or ring system, whereby the terms “annulated” or “annelated” are also used by those skilled in the art to designate this kind of attachment.

### Compositions

In one aspect, the invention provides for a compound of any one formula selected from XVI through XXX, wherein A1 through A31 are each independently R1 or —H, with the proviso that at least one substituent on each of said formula XVI through XXX is R1; and salts, co-crystals, geometric isomers and stereoisomers thereof.

In one embodiment, said compound is a compound of formula (XVI), wherein A1 and A2 are independently selected from R1 or —H, wherein A1 and A2 are not both —H; and salts, co-crystals, geometric isomers and stereoisomers thereof. In a further embodiment, A1 is R1 and A2 is —H. In a further embodiment, A2 is R1 and A1 is —H. In a further embodiment, with respect to the 3 and 3′ positions of the parent astaxanthin, the compound is an isomer selected from the group consisting of: (3S, 3′S), (3S, 3′R), (3R, 3′S) and (3R, 3′R), or a mixture thereof, e.g. 1:2:1 3S,3′S:meso(3S,3′R, identical to 3R,3′S):3R,3′R. In a preferred embodiment, the isomer is (3S, 3′S). In a further embodiment, with respect to the isomerism around the C═C double bonds in the polyene chain, the compound is selected from the group consisting of: all-trans, 9-cis, 13-cis and 15-cis (with trans C═C in the remaining positions).

In another embodiment, said compound is a compound of formula (XVII), wherein A3 and A4 are independently selected from R1 or —H, wherein A3 and A4 are not both —H; and salts, co-crystals, geometric isomers and stereoisomers thereof. In a further embodiment, A3 is R1 and A4 is —H. In a further embodiment, A4 is R1 and A3 is —H

In another embodiment, said compound is a compound of formula (XVIII), wherein A5 and A6 are independently selected from R1 or —H, wherein A5 and A6 are not both —H; and salts, co-crystals, geometric isomers and stereoisomers thereof. In a further embodiment, A5 is R1 and A6 is —H. In a further embodiment, A6 is R1 and A5 is —H.

In another embodiment, said compound is a compound of formula (XIX), wherein A7 and A8 are independently selected from R1 or —H, wherein A5 and A6 are not both —H; and salts, co-crystals, geometric isomers and stereoisomers thereof. In a further embodiment, A7 is R1 and A8 is —H. In a further embodiment, A8 is R1 and A7 is —H.

In another embodiment, said compound is a compound of formula (XX), wherein A9 and A10 are independently selected from R1 or —H, wherein A9 and A10 are not both —H; and salts, co-crystals, geometric isomers and stereoisomers thereof. In a further embodiment, A9 is R1 and A10 is —H. In a further embodiment, A9 is —H and A10 is R1.

In another embodiment, said compound is a compound of formula (XXI), wherein A11 and A12 are independently selected from R1 or —H, wherein A11 and A12 are not both —H; and salts, co-crystals, geometric isomers and stereoisomers thereof. In a further embodiment, A11 is R1 and A12 is —H. In a further embodiment, A11 is —H and A12 is R1.

In another embodiment, said compound is a compound of formula (XXII), wherein A13 and A14 are independently selected from R1 or —H, wherein A13 and A14 are not both —H; and salts, co-crystals, geometric isomers and stereoisomers thereof. In a further embodiment, A13 is R1 and A14 is —H. In a further embodiment, A13 is —H and A14 is R1.

In another embodiment, said compound is a compound of formula (XXIII), wherein A15 and A16 are independently selected from R1 or —H, wherein A15 and A16 are not both —H; and salts, co-crystals, geometric isomers and stereoisomers thereof. In a further embodiment, A15 is R1 and A16 is —H. In a further embodiment, A15 is —H and A16 is R1.

In another embodiment, said compound is a compound of formula (XXIV), wherein A17 and A18 are independently selected from R1 or —H, wherein A17 and A18 are not both —H; and salts, co-crystals, geometric isomers and stereoisomers thereof. In a further embodiment, A17 is R1 and A18 is —H. In a further embodiment, A17 is —H and A18 is R1.

In another embodiment, said compound is a compound of formula (XXV), wherein A19 is R1; and salts, co-crystals, geometric isomers and stereoisomers thereof.

In another embodiment, said compound is a compound of formula (XXVI), wherein A20 and A21 are independently selected from R1 or —H, wherein A20 and A21 are not both —H; and salts, co-crystals, geometric isomers and stereoisomers thereof. In a further embodiment, A20 is R1 and A21 is —H. In a further embodiment, A20 is —H and A21 is R1.

In another embodiment, said compound is a compound of formula (XXVII), wherein A22 and A23 are independently selected from R1 or —H, wherein A22 and A23 are not both —H; and salts, co-crystals, geometric isomers and stereoisomers thereof. In a further embodiment, A22 is R1 and A23 is —H. In a further embodiment, A22 is —H and A23 is R1.

In another embodiment, said compound is a compound of formula (XXVIII), wherein A24, A26 and A26 are independently selected from R1 or —H, wherein A24, A25 and A26 are not all —H; and salts, co-crystals, geometric isomers and stereoisomers thereof. In a further embodiment, A24 and A25 are independently selected from R1 and A26 is —H. In a further embodiment, A24 and A26 are independently selected from R1 and A25 is —H. In a further embodiment, A25 and A26 are independently selected from R1 and A24 is —H.

In another embodiment, said compound is a compound of formula (XXIX), wherein A27, A28 and A29 are independently selected from R1 or —H, wherein A27, A28 and A29 are not all —H; and salts, co-crystals, geometric isomers and stereoisomers thereof. In a further embodiment, A27 and A28 are independently selected from R1 and A29 is —H. In a further embodiment, A27 and A29 are independently selected from R1 and A28 is —H. In a further embodiment, A28 and A29 are independently selected from R1 and A27 is —H.

In another embodiment, said compound is a compound of formula (XXX), wherein A30 and A31 are independently selected from R1 or —H, wherein A30 and A31 are not both —H; and salts, co-crystals, geometric isomers and stereoisomers thereof. In a further embodiment, A30 is R1 and A31 is —H. In a further embodiment, A30 is —H and A31 is R1.

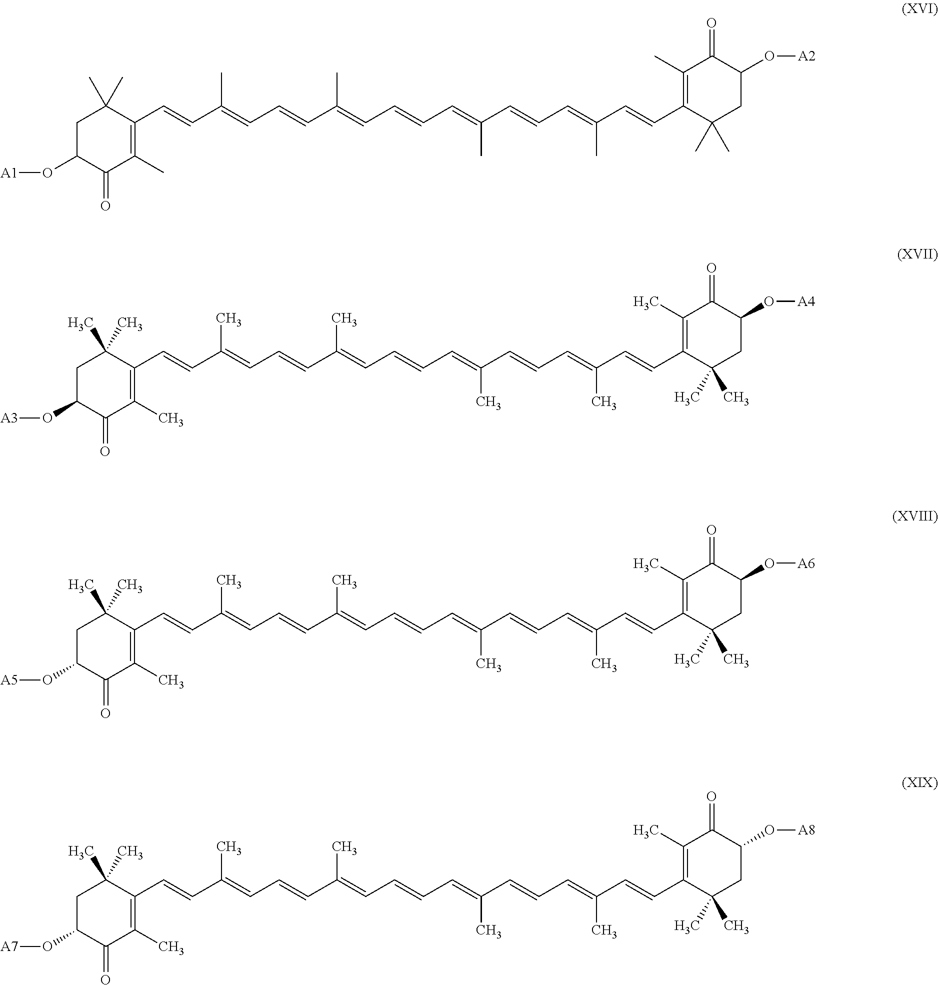

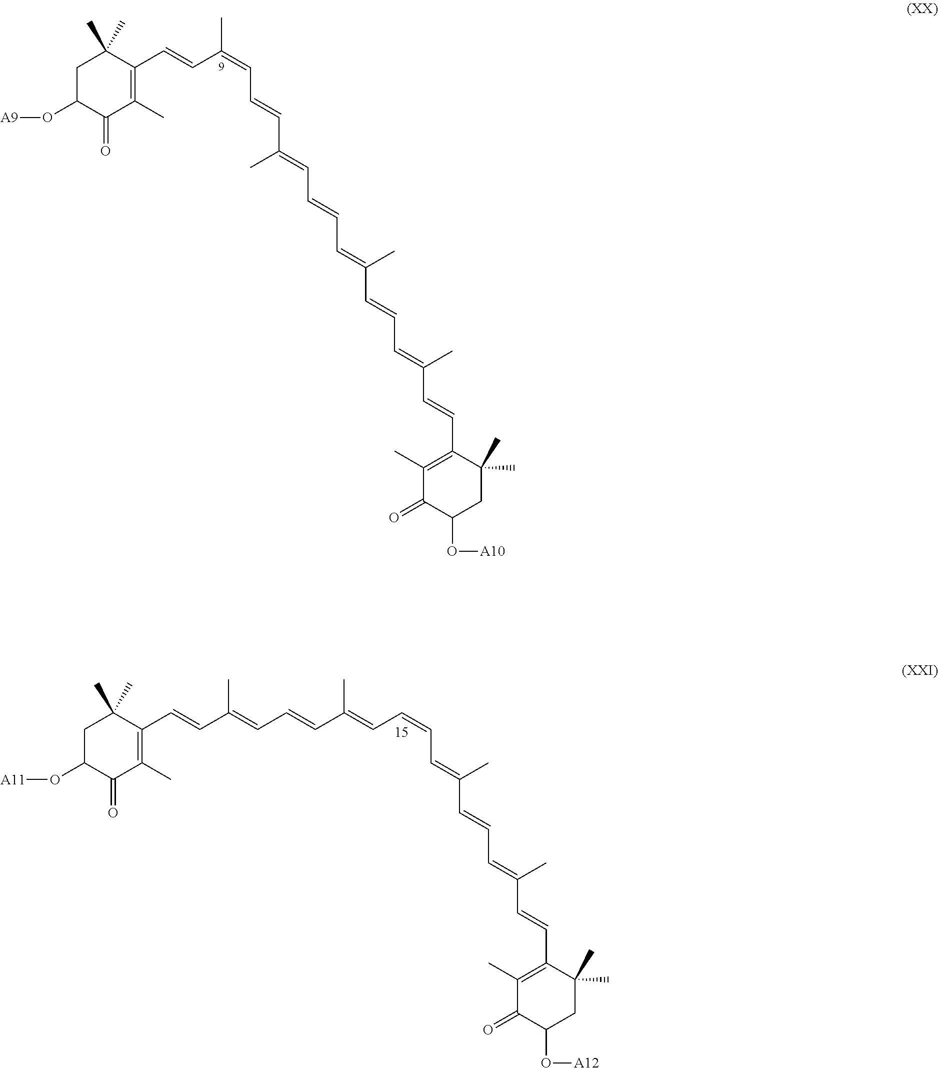

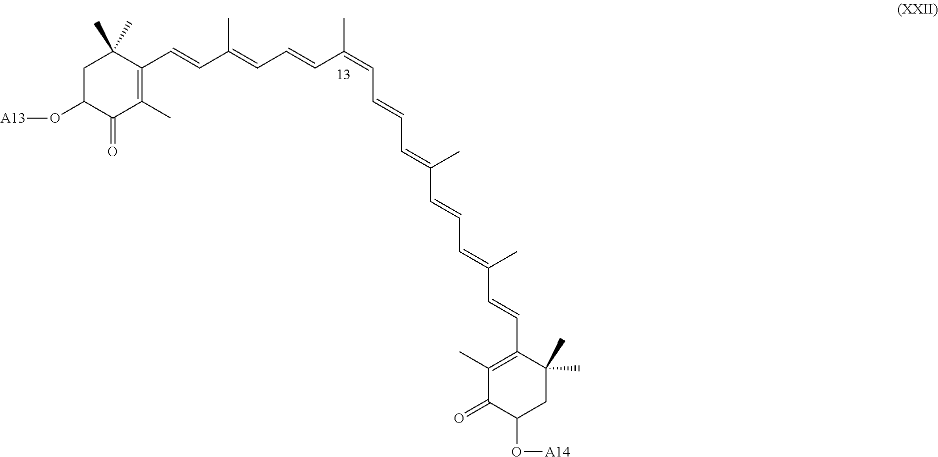

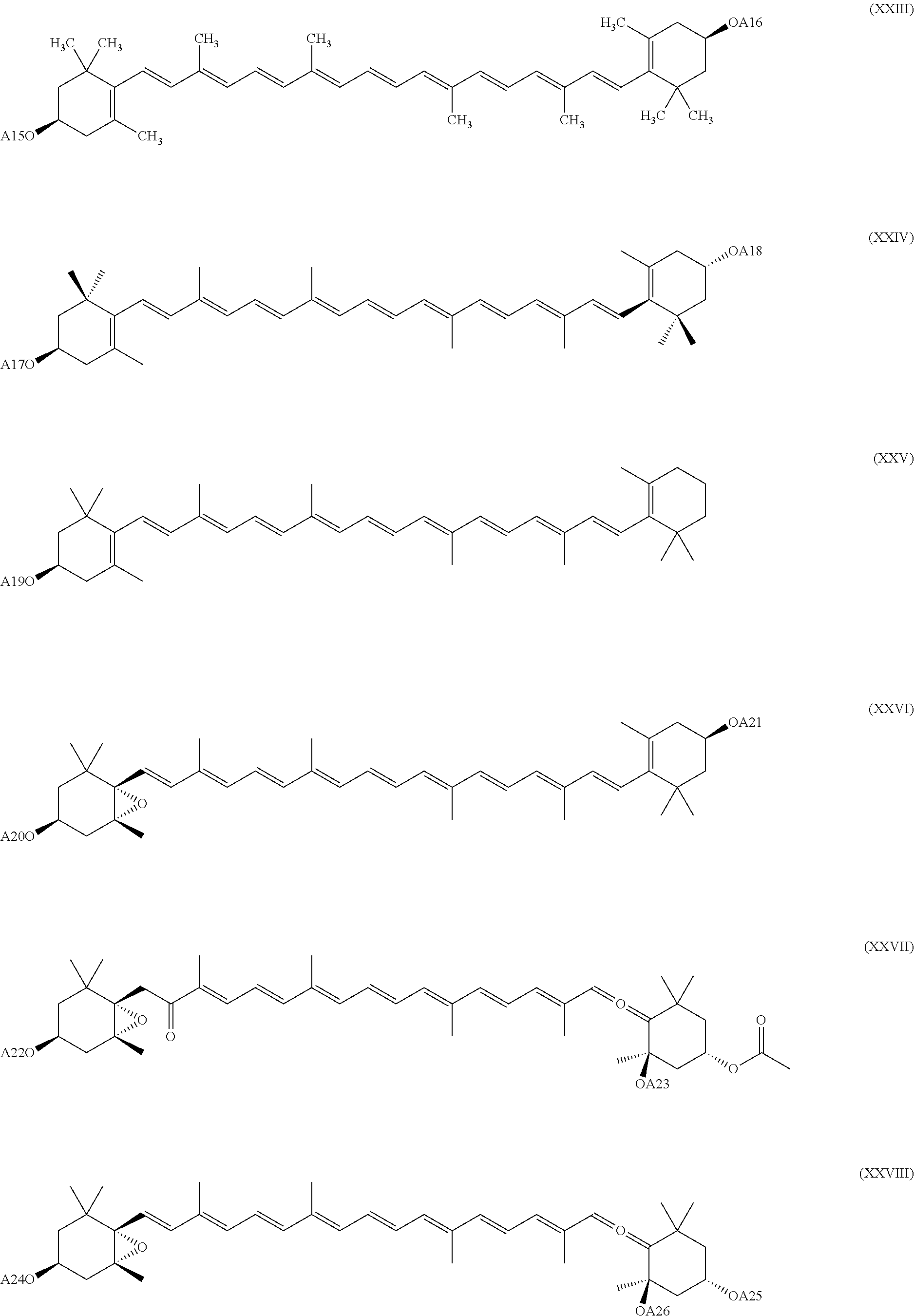

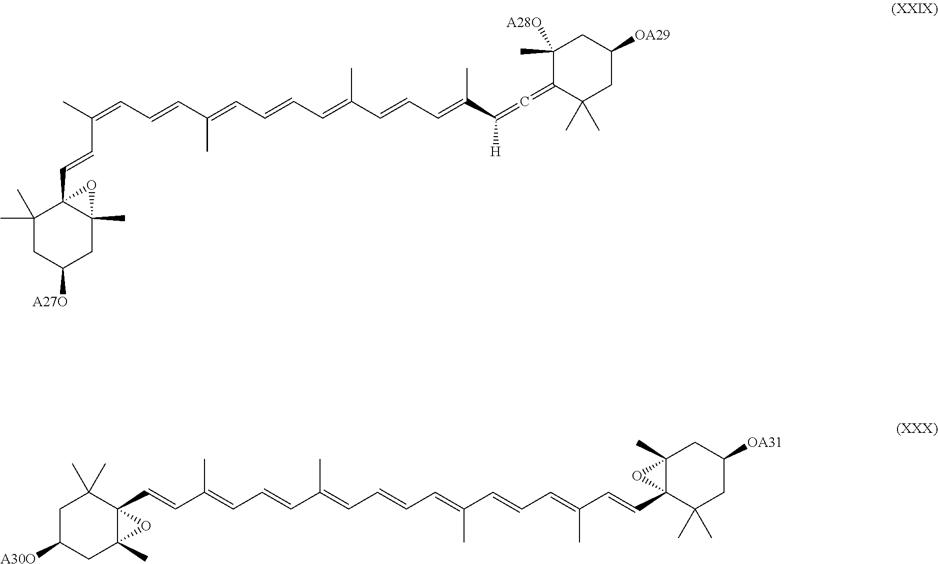

In one embodiment, R1 is a radical selected from: acyl or ester; straight or branched-chain, substituted or unsubstituted, saturated or unsaturated; amide, imide; ester; or —NO₂.

In another embodiment, R1 is —C(═O)R2, wherein R2 is a radical selected from: —H; —OH; a saturated or unsaturated, unsubstituted or substituted aliphatic radical; straight-chain, substituted or unsubstituted C₁-C₁₀-alkyl; branched-chain, substituted or unsubstituted C₃-C₁₀ alkyl.

In another embodiment, R1, together with the xanthophyll oxygen to which it is bound, is an ester of an acid selected from: acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric, or galacturonic acid.

In another embodiment, R1 is —C(O)CH(R3)NH₂, —C(O)CH(R3)NHC(O)CH(R4)NH₂, or —C(O)CH(R3)NHC(O)CH(R4)NHC(O)CH(R5)NH₂; wherein R3, R4, and R5 are independently an amino acid side chain group. In one embodiment, R3, R4, and R5 are independently selected from an amino acid side chain of valine, sarcosine, leucine, glutamine, tryptophan, tyrosine, alanine or 4-(4-aminophenyl)butyric acid.

In another embodiment, R1 is selected from: —C(O)(CH₂)₁₋₁₀C(O)OH; or —C(O)(CH₂)₁₋₁₀N(R6)R6, wherein each R6 is independently —H, alkyl, or together form a saturated, unsaturated, or substituted ring. In a further embodiment, said alkyl is C₁-C₅-alkyl or C₁-C₁₀-alkyl.

In one embodiment, R1 is selected from the group consisting of: acyl; —C(═O)H; —C(═O)R7; —C(═O)—(CHR8)ᵣ—R7, —C(═O)—(CHR8)ᵣ—R7, —C(═O)R7, —C(═O)OR7,—C(═O)OH, —P(═O)(OR7)₂, —P(═O) (OH)(OR7), —P(═O)(OH)₂, —C(═O)NH₂, —C(═O)NHR7, —C(═O)N(R7)R7,—S(═O)R7, —S(═O)₂R7, —C(═S)OR7 or —C(═S)R7; where r is 1, 2, 3, 4, 5, 6, 7 or 8; wherein R7, is, in each case independently, a saturated or unsaturated, unsubstituted or substituted aliphatic radical; or an unsubstituted or substituted, aryl or heteroaryl, which may be condensed with an unsubstituted or substituted mono- or polycyclic ring system and/or may be bonded via an unsubstituted or substituted alkandiyl; and, wherein R8 is —H or a saturated or unsaturated, unsubstituted or substituted alkandiyl, alkendiyl or alkyndiyl.

If one or more of the residues R7 or R8 represents or comprises an aryl or heteroaryl, such aryl or heteroaryl may optionally be substituted with 1, 2, 3, 4, or 5 substituent(s) independently selected from the group consisting of: —C₁₋₆-perfluoralkyl, —C₁₋₆-alkyl substituted with one or more methoxy and/or ethoxy groups, —C₁₋₆-alkyl, —C₁₋₆-alkyl substituted with one or more hydroxy groups, —C₁₋₆-alkyl substituted with one or more chlorine atoms, —OC₁₋₆-alkyl, —OC₁₋₆-alkyl substituted with one or more methoxy and/or ethoxy groups, —SC₁₋₆-alkyl, —C(═O)OH, —C(═O)OC₁₋₆-alkyl, —OC(═O)C₁₋₆-alkyl, —F, —Cl, —Br, —I, —CN, —OCF₃, —OC₂F₅, —OC₃F₇, —OC₄F₉, —SCF₃, —SCF₃, —SCF₂H, —SCFH₂, —OH, —SH, —SO₃H, —NHC(═O)C₁₋₆-alkyl, —N(C₁₋₆-alkyl)C(═O)C₁₋₆-alkyl, —NO₂, —CHO, —C(═O)C₁₋₆-alkyl, —C(═O)—C₁₋₆-perfluoroalkyl, —C(═S)—NH—C₁₋₆-alkyl, —CF₂H, —CFH₂, —C(═O)N(R9)R10, —C(═O)NHN(R11)(R12), —S(═O)C₁₋₆-alkyl, —S(═O)₂C₁₋₆-alkyl, —S(═O)₂-phenyl, —S(═O)-phenyl, —C₁₋₅-alkylene-SC₁₋₆-alkyl, —C₁₋₅-alkylene-S(═O)C₁₋₆-alkyl, —C₁₋₅-alkylene-S(═O)₂C₁₋₆-alkyl, —N(R13)R14, —C₁₋₅-alkylene-N(R13)R14, S(═O)NH₂, —S(═O)₂NHC₁₋₆-alkyl, —S(═O)₂NH-phenyl, —NHS(═O)₂C₁₋₆-alkyl, —OS(═O)C₁₋₆-alkyl-, —OS(═O)-phenyl, —CS(═O)-benzyl, —OS(═C)₂C₁₋₆-alkyl, —OS(═O)₂-phenyl, —O—S(═O)₂-benzyl, —N⁺(C₁₋₆-alkyl)₃, —O-phenyl, —O-benzyl, —S-phenyl, —S-benzyl, —C(═O)O-benzyl, —C(═O)O-phenyl, —C(═O)-benzyl, —C(═O)-phenyl, cyclopropyl, cyclobutyl, cyclopentyl, cyclohexyl, pyrrolidinyl, piperidinyl, phenyl, thiophenyl and benzyl; wherein in each case the cyclic moieties cyclopropyl, cyclobutyl, cyclopentyl, cyclohexyl, pyrrolidinyl, piperidinyl, phenyl, thiophenyl and benzyl may be optionally substituted with 1, 2, 3, 4 or 5 substituent(s) independently selected from the group consisting of: —F, —Cl, —Br, —I, —OH, —CF₃, —CN, —NO₂, —C₁₋₆-alkyl, —OC₁₋₆-alkyl, —OCF₃, and —SCF₃.

R9, R10, R13 and R14, are each independently selected from: —H, —C₁₋₆-alkyl, or R9 and R10, together with the nitrogen atom to which they are bound, form a functional group consisting of: pyrrolidinyl, imidazolidinyl, piperazinyl, piperidinyl, thiomorpholinyl, morpholinyl, azepanyl and diazepanyl, which may be at least mono-substituted with one or more identical or different C₁₋₆ alkyl radicals.

R11 and R12, independently of one another, represent —H, —C₁₋₆-alkyl, —C(═O)OC₁₋₆-alkyl, C₃₋₈-cycloalkyl, —C₁₋₅-alkylene-C₃₋₈-cycloalkyl, —C₁₋₆-alkylene-OC₁₋₆-alkyl or —C₁₋₆-alkyl substituted with one or more hydroxy groups, or R11 and R12, together with the nitrogen atom to which they are bound, for a functional group selected from the group consisting of: pyrrolidinyl, imidazolidinyl, piperazinyl, piperidinyl, thiomorpholinyl, morpholinyl, azepanyl and diazepanyl, which may be at least mono-substituted with one or more substituents independently selected from the group consisting of: —C₁₋₆-alkyl, —C(═O)C₁₋₆-alkyl, —C(═O)OC₁₋₆-alkyl, —C(═O)NHC₁₋₆-alkyl, —C(═S)NHC₁₋₆-alkyl, oxo (═O), —C₁₋₆-alkyl substituted with one or more hydroxy groups, —C₁₋₆-alkylene-OC₁₋₆-alkyl, and —C(═O)NH₂.

Aryl and heteroaryl radicals may optionally be substituted with 1, 2, 3, 4 or 5 substituent(s) independently selected from the group consisting of —CF₃, —C₂F₅, —C₃F₂, —C₄F₉, —CH₂Cl, —CHCl₂, —C₂H₄Cl, methyl, ethyl, n-propyl, isopropyl, n-butyl, isobutyl, 2-butyl, tert-butyl, n-pentyl, 2-pentyl, n-hexyl, —CH2—OH, —CH₂—CH₂—OH, —CH₂—CH₂—CH₂—OH, —O—CH₂—O—CH₃, —O—CH₂—CH₂—O—CH₃, —O—CH₂—O—C₂H₅, —C(OCH₃)(C₂H₅)₂, —C(OCH₃)(CH₃)₂, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —C(═O)—OH, —C(═O)—O—CH₃, —C(═O)—O—C₂H₅, —C(═O)—O—C₃H₇, —C(═O)—O—C(CH₃)₃, —O—C(═O)—CH₃, —O—C(═O)—C₂H₅, —O—C(═O)—CH(CH₃)₂, —O—C(═O)—CH₂—CH₂—CH₃, —O—C(═O)—C(CH₃)₃, F, Cl, Br, I, —CN, —OCF₃, —O—C₂F₅, —O—C₃F₇, —O—C₄F₉, —SCF₃, —SCF₂H, —S CFH₂, —OH, —SH, —SO₃H, —NH—C(═O)—CH₃, —NH—C(═O)—C₂H₅, —NH—C(═O)—C(CH₃)₃, —NO₂, —CHO, —C(═O)—CH₃, —C(═O)—C₂H₅, —C(═O)—C(CH₃)₃, —C(═O)—CF₃, —C(═O)—C₂F₅, —C(═O)—C₃F₇, —C(═S)—NH—CH₃, —C(═S)—NH—C₂H₅, —CF₂H, —CFH₂, —C(═O)—NH₂, —C(═O)—NH—CH₃, —C(═O)—NH—C₂H₅, —C(═O)—NH—C₃H₇, —C(═O)—N(CH₃)₂, —C(═O)—N(C₂H₅)₂, —C(═O)—NH—NH—CH₃, —C(═O)—NH—NH—C₂H₅, —C(═O)—NH—NH₂, —C(═O)—NH—N(CH₃)₂, —S(═O)—CH₃, —S(═O)—C₂H₅, —S(═O)—C₃H₇, —S(═O)₂—CH₃, —S(═O)₂—C₂H₅, —S(═O)₂—C₃H₇, —S(═O)₂-phenyl, —NH₂, —NH—CH₃, —NH—C₂H₅, —N(CH₃)₂, —N(C₂H₅)₂, —CH₂—N(CH₃)₂, —(CH₂)-morpholinyl, —(CH₂)-piperidinyl, —(CH₂)-piperazinyl, —(CH₂)—N(C₂H₅)₂, —CH₂—N(C₃H₂)₂, —CH₂—N(C₄H₉)₂, —CH₂—N(CH₃)(C₂H₅), —S(═O)—NH₂, —S(═O)₂—NH—CH₃, —S(═O)₂—NH-phenyl, —NH—S(═O)₂—CH₃, —N⁺(CH₃)₃, —N⁺(C₂H₅)₃, —N⁺[C(CH₃)₃]₃—S(═O)—C(CH₃)₃, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, cyclopropyl, cyclobutyl, cyclopentyl, cyclohexyl, pyrrolidinyl, piperidinyl, phenyl, thiophenyl and benzyl, whereby said thiophenyl radical can be substituted with 1, 2 or 3 substituent(s) independently selected from the group consisting of —F, —Cl, —Br, methyl, ethyl and n-propyl.

In one embodiment, aryl radicals are substituted phenyl or substituted naphthyl (1- and 2-naphthyl).

In another embodiment, the heteroatoms which are present as ring members in the heteroaryl radical may, unless defined otherwise, independently be selected from the group consisting of nitrogen, oxygen and sulfur. More preferably a heteroaryl radical is 5- to 14-membered and may comprise 1, 2, 3 or 4 heteroatoms independently selected from the group consisting of: nitrogen, oxygen and sulfur.

In another embodiment, heteroaryl radicals which are unsubstituted or at least mono-substituted are pyridinyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, indolyl, isoindolyl, pyrimidinyl, pyrazinyl, quinolinyl, isoquinolinyl, benzo[b]furanyl, benzo[b]thiophenyl, benzo[2,1,3]thiadiazolyl, [1,2,3]-benzothiadiazolyl, [2,1,3]-benzoxadiazolyl, [1,2,3]-benzoxadiazolyl, benzoxazolyl, benzthiazolyl, benzisoxazolyl, benzisothiazolyl, imidazo[2,1-b]thiazolyl, 2H-chromenyl, pyranyl, indazolyl, and quinazolinyl.

In another embodiment, aryl and heteroaryl radicals which are condensed with a mono- or polycyclic ring system are [1,3]-benzodioxolyl, [1,4]-benzodioxanyl, [1,2,3,4]-tetrahydronaphthyl, (2,3)-dihydro-1H-cyclopenta[b]indolyl, [1,2,3,4]-tetrahydroquinolinyl, [1,2,3,4]-tetrahydroisoquinolinyl, [1,2,3,4]-tetrahydroquinazolinyl and [3,4]-dihydro-2H-benzo[1,4]oxazinyl.

In other embodiments, where one or more R7 or R8 is or comprises a saturated or unsaturated, cycloaliphatic radical with at least one heteroatom as a ring member, R7 or R8 is selected from: a C₃₋₁₈ cycloaliphatic radical, a heterocyclic ring radical, preferably a 4- to 10-membered heterocyclic ring, C₃₋₁₆ cycloalkyl, C₄₋₁₆ cycloalkenyl, C₄₋₁₆ heterocycloalkyl, or C₅₋₁₆ heterocycloalkenyl; which may be substituted, unless defined otherwise. Preferably said cycloaliphatic radical, heterocyclic ring radical, C₃₋₁₆ cycloalkyl, C₄₋₁₆ cycloalkenyl, C₄₋₁₆ heterocycloalkyl, or C₅₋₁₆ heterocycloalkenyl, may in each case be substituted with 1, 2, 3, 4 or 5 substituent(s) independently selected from the group consisting of: oxo (═O), thioxo (═S), —C₁₋₆-perfluoralkyl, —C₁₋₆ alkyl, —C₁₋₆-alkyl substituted with one or more hydroxy groups, —C₁₋₆-alkyl substituted with one or more chlorine atoms, —C₁₋₆-alkyl substituted with one or more methoxy and/or ethoxy groups, —O—C₁₋₆-alkyl, —O—C₁₋₆-alkyl substituted with one or more methoxy and/or ethoxy groups, —S—C₁₋₆-alkyl, —C(═O)—OH, —C(═O)—O—C₁₋₆-alkyl, —OC(═O)C₁₋₆-alkyl, —F, —Cl, —Br, —I, —CN, —OCF₃, —O—C₂F₅, —O-C₃F₇, —O—C₄F₉, —SCF₃, —SCF₂H, —SCFH₂, —OH, —SH, —SO₃H, —NHC(═O)C₁₋₆-alkyl, —N(C₁₋₆-alkyl)-C(═O)C₁₋₆-alkyl, —NO₂, —CHO, —C(═O)C₁₋₆-alkyl, —C(═O)C₁₋₆-perfluoroalkyl, —C(═S)NHC₁₋₆-alkyl, —CF₂H, —CFH₂, —C(═O)N(R9)R10,—C(═O)NHN(R11)R11,—S(═O)C₁₋₆-alkyl, —S(═O)₂C₁₋₆-alkyl, —S(═O)₂-phenyl, —C₁₋₅-alkylene-SC₁₋₆-alkyl, —C₁₋₅-alkylene-S(═O)C₁₋₆-alkyl, —C₁₋₅-alkylene-S(═O)₂C₁₋₆-alkyl, —N(R13)R14, —C₁₋₅-alkylene-N(R13)R14, S(═O)NH₂, —S(═O)₂NHC₁₋₆-alkyl, —S(═O)₂NH-phenyl, —NHS(═O)₂C₁₋₆-alkyl, —O-benzyl, —Ophenyl, cyclopropyl, cyclobutyl, cyclopentyl, cyclohexyl, pyrrolidinyl, piperidinyl, phenyl, thiophenyl and benzyl; wherein cyclic moieties cyclopropyl, cyclobutyl, cyclopentyl, cyclohexyl, pyrrolidinyl, piperidinyl, phenyl, thiophenyl and benzyl may optionally and independently be substituted with 1, 2, 3, 4 or 5 substituent(s) independently selected from the group consisting of: —F, —Cl, —Br, —I, —OH, —CF₃, —CN, —NO₂, —C₁₋₆-alkyl, —O—C₁₋₆-alkyl, —O—CF₃ and —S—CF₃; and wherein R9, R10, R13 and R14, independently of one another, represent —H or —C₁₋₆-alkyl or R9 and R10, together with the nitrogen atom to which they are bound, form a radical selected from the group consisting of pyrrolidinyl, imidazolidinyl, piperazinyl, piperidinyl, thiomorpholinyl, morpholinyl, azepanyl and diazepanyl which may be at least mono-substituted with one or more identical or different C₁₋₆ alkyl radicals and whereby Rn. and R12, are, independently, selected from: —H, —C₁₋₆ alkyl, —C(═O)OC₁₋₆-alkyl, C₃₋₈-cycloalkyl, —C₁₋₅-alkylene-C₃₋₈-cycloalkyl, —C₁₋₆-alkylene-OC₁₋₆-alkyl or —C₁₋₆-alkyl substituted with one or more hydroxy groups or R11 and R12, together with the nitrogen atom to which they are bound form a group that may be selected from: pyrrolidinyl, imidazolidinyl, piperazinyl, piperidinyl, thiomorpholinyl, morpholinyl, azepanyl, or diazepanyl, which may be at least mono-substituted with one or more substituents independently selected from: —C₁₋₆-alkyl, —C(═O)C₁₋₆-alkyl, —C(═O)OC₁₋₆-alkyl, —C(═O)NHC₁₋₆-alkyl, —C(═S)NHC₁₋₆-alkyl, oxo (═O), —C₁₋₆-alkyl substituted with one or more hydroxy groups, —(C₁₋₆-alkylene)-OC₁₋₆-alkyl or —C(═O)—NH₂.

In other embodiments, said cycloaliphatic radicals, heterocyclic rings, C₃₋₁₆ cycloalkyl radicals, C₄₋₁₆ cycloalkenyl radicals, C₄₋₁₆ heterocycloalkyl radicals, or C₅₋₁₆ heterocycloalkenyl radicals, may optionally be substituted with 1, 2, 3, 4 or 5 substituent(s) independently selected from the group consisting of oxo (═O), thioxo (═S), —CF₃, —C₂F₅, —C₃F₂, —C₄F₉, —CH₂Cl, —CHCl₂, —C₂H₄Cl, methyl, ethyl, n-propyl, isopropyl, n-butyl, isobutyl, 2-butyl, tert-butyl, n-pentyl, 2-pentyl, n-hexyl, —CH₂—OH, —CH₂—CH₂—OH, —CH₂—CH₂—CH₂—OH, —O—CH₂—O—CH₃, —O—CH₂—CH₂—O—CH₃, —O—CH₂—O—C₂H₅, —C(OCH₃)(C₂H₅)₂, —C(OCH₃)(CH₃)₂, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —C(═O)—OH, —C(═O)—O—CH₃, —C(═O)—O—C₂H₅, —C(═O)—O—C₃H₇, —C(═O)—O—C(CH₃)₃, —O—C(═O)—CH₃, —O—C(═O)—C₂H₅, —O—C(═O)—CH(CH₃)₂, —O—C(═O)—CH₂—CH₂—CH₃, —O—C(═O)—C(CH₃)₃, F, Cl, Br, I, —CN, —OCF₃, —O—C₂F₅, —O—C₃F₇, —O—C₄F₉, —SCF₃, —SCF₂H, —SCFH₂, —OH, —SH, —SO₃H, —NH—C(═O)—CH₃, —NH—C(═O)—C₂H₅, —NH—C(═O)—C(CH₃)₃, —NO₂, —CHO, —C(═O)—CH₃, —C(═O)—C₂H₅, —C(═O)—C(CH₃)₃, —C(═O)—CF₃, —C(═O)—C₂F₅, —C(═O)—C₃F₇, —C(═S)—NH—CH₃, —C(═S)—NH—C₂H₅, —CF₂H, —CFH₂, —C(═O)—NH₂, —C(═O)—NH—CH₃, —C(═O)—NH—C₂H₅, —C(═O)—NH—C₃H₇, —C(═O)—N(CH₃)₂, —C(═O)—N(C₂H₅)₂, —C(═O)—NH—NH—CH₃, —C(═O)—NH—NH—C₂H₅, —C(═O)—NH—NH₂, —C(═O)—NH—N(CH₃)₂, —S(═O)—CH₃, —S(═O)—C₂H₅, —S(═O)—C₃H₇, —S(═O)₂—CH₃, —S(═O)₂—C₂H₅, —S(═O)₂—C₃H₇, —S(═O)₂-phenyl, —NH₂, —NH—CH₃, —NH—C₂H₅, —N(CH₃)₂, —N(C₂H₅)₂, —CH₂—N(CH₃)₂, —(CH₂)-morpholinyl, —(CH₂)-piperidinyl, —(CH₂)-piperazinyl, —(CH₂)—N(C₂H₅)₂, —CH₂—N(C₃H₇)₂, —CH₂—N(C₄H₉)₂, —CH₂—N(CH₃)(C₂H₅), —S(═O)—NH₂, —S(═O)₂—NH—CH₃, —S(═O)₂—NH-phenyl, —NH—S(═O)₂—CH₃, —O-Benzyl, —O-Phenyl, cyclopropyl, cyclobutyl, cyclopentyl, cyclohexyl, pyrrolidinyl, piperidinyl, phenyl, thiophenyl and benzyl, whereby said thiophenyl radical can be substituted with 1, 2 or 3 substituents independently selected from the group consisting of F, Cl, Br, methyl, ethyl and n-propyl.

If one or more of the residues in R1 represents or comprises a cycloaliphatic radical, preferably a C₃₋₁₆ cycloaliphatic radical, which contains one or more heteroatoms as ring members, unless defined otherwise, each of these heteroatoms is independently selected from nitrogen, oxygen or sulfur. More preferably a cycloaliphatic group may optionally contain 1, 2, 3 or 4 heteroatom(s) independently selected from the group consisting of N, O and S as ring members.

Suitable saturated or unsaturated, optionally at least one heteroatom as ring member containing cycloaliphatic radicals may preferably be selected from the group consisting of cyclopropyl, cyclobutyl, cyclopentyl, cyclohexyl, cycloheptyl, cyclooctyl, cyclononyl, cyclodecyl, cycloundecyl, cyclododecyl, cyclotridecyl, cyclotetradecyl, cyclopentenyl, cyclohexenyl, cycloheptenyl, cyclooctenyl, pyrrolidinyl, piperidinyl, piperazinyl, homopiperazinyl, morpholinyl, aziridinyl, azetidinyl, imidazolidinyl, thiomorpholinyl, pyrazolidinyl, tetrahydrofuranyl, tetrahydrothiophenyl, azepanyl, diazepanyl, azocanyl, (2,5)-dihydrofuranyl, (2,5)-dihydrothiophenyl, (2,3)-dihydrofuranyl, (2,3)-dihydrofuranyl, (2,5)-dihydro-1H-pyrrolyl, (2,3)-dihydro-1H-pyrrolyl, tetrahydrothiopyranyl, tetrahydropyranyl, (3,4)-dihydro-2H-pyranyl, (3,4)-dihydro-2H-thiopyranyl, (1,2,3,6)-tetrahydropyridinyl, (1,2,3,4)-tetrahydropyridinyl, (1,2,5,6)-tetrahydropyridinyl, [1,3]-oxazinanyl, hexahydropyrimidinyl, (5,6)-dihydro-4H-pyrimidinyl, oxazolidinyl, (1,3)-dioxanyl, (1,4)-dioxanyl and (1,3)-dioxolanyl.

Suitable saturated or unsaturated, optionally at least one heteroatom as ring member containing cycloaliphatic radicals which are condensed with an unsubstituted or at least mono-substituted mono- or polycyclic ring system may preferably be selected from the group consisting of indolinyl, isoindolinyl, decahydronaphthyl, (1,2,3,4)-tetrahydroquinolinyl, (1,2,3,4)-tetrahydroisoquinolinyl, (1,2,3,4)-tetrahydronaphthyl, octahydro-cyclopenta[c]pyrrolyl, (1,3,4,7,9a)-hexahydro-2H-quinolizinyl, (1,2,3,5,6,8a)-hexahydro-indolizinyl, decahydroquinolinyl, dodecahydro-carbazolyl, 9H-carbazolyl, decahydroisoquinolinyl, (6,7)-dihydro-4H-thieno[3,2-c]pyridinyl, (2,3)-dihydro-1H-benzo[de]isoquinolinyl and (1,2,3,4)-tetrahydroquinoxazlinyl.

In another embodiment, a cycloaliphatic radical, a C₃₋₁₆ cycloalkyl radical, a C₄₋₁₆ cycloalkenyl radical, a C₄₋₁₆ heterocycloalkyl radical or a C₅₋₁₆ heterocycloalkenyl radical may be bridged by 1, 2 or 3 unsubstituted or at least mono-substituted alkylene group(s).

Suitable saturated or unsaturated, cycloaliphatic radicals, optionally containing at least one heteroatom as ring member, which are bridged by at least one unsubstituted or at least mono-substituted alkylene group may preferably be selected from the group consisting of adamantyl, bicyclo[2.2.1]heptyl, bicyclo[3.1.1]heptyl, norbornenyl and 8-aza-bicyclo[3.2.1]octyl.

A suitable saturated or unsaturated, optionally at least one heteroatom as ring member containing cycloaliphatic radical which together with a saturated or unsaturated, unsubstituted or at least mono-substituted cycloaliphatic radical forms a spirocyclic residue via a common ring atom is 8-aza-spiro[4.5]decanyl.

A mono- or poly-cyclic ring system as used herein includes a mono- or polycyclic hydrocarbon ring system, preferably a mono- or bicyclic ring system, that may be saturated, unsaturated or aromatic. Each of its different rings may show a different degree of saturation, i.e. they may be saturated, unsaturated or aromatic. Optionally each of the rings of the mono- or bicyclic ring system may contain one or more, preferably 1, 2 or 3, heteroatom(s) as ring member(s), which may be identical or different and which can preferably be selected from the group consisting of nitrogen, oxygen and sulfur. The rings of the mono-or bicyclic ring system are preferably 5-, 6- or 7-membered.

If one or more of the residues R7 or R8 comprises a mono- or poly-cyclic ring system, which may be substituted, unless defined otherwise, preferably said mono- or polycyclic ring system may optionally be substituted with 1, 2, 3, 4 or 5 substituent(s), independently selected from the group consisting of: oxo (═O), thioxo (═S), —C₁₋₆-perfluoralkyl, —C₁₋₆-alkyl, —C₁₋₆-alkyl substituted with one or more hydroxy groups, —C₁₋₆-alkyl substituted with one or more chlorine atoms, —C₁₋₆-alkyl substituted with one or more methoxy and/or ethoxy groups, —OC₁₋₆-alkyl, —OC₁₋₆-alkyl substituted with one or more methoxy and/or ethoxy groups, —SC₁₋₆-alkyl, —C(═O)OH, —C(═O)OC₁₋₆-alkyl, —OC(═O)C₁₋₆-alkyl, —F, —Cl, —Br, —I, —CN, —OCF₃, —O—C₂F₅, —O—C₃F₇, —O—C₄F₉, —SCF₃, —SCF₂H, —SCFH₂, —OH, —SH, —SO₃H, —NHC(═O)—C₁₋₆-alkyl, —N(C₁₋₆-alkyl)-C(═O)—C₁₋₆-alkyl, —NO₂, —CHO, —C(═O)—C₁₋₆-alkyl, —C(═O)C₁₋₆-perfluoroalkyl, —C(═S)NH—C₁₋₆-alkyl, —CF₂H, —CFH₂, —C(═O)—NN(R9)R11, —C(═O)—NHN(R11)R12, —S(═O)—C₁₋₆-alkyl, —S(═O)₂—C₁₋₆-alkyl, —S(═O)₂-phenyl, —(C₁₋₅-alkylene)-S—C₁₋₆-alkyl, —C₁₋₅-alkylene-S(═O)—C₁₋₆-alkyl, —C₁₋₅-alkylene-S(═O)₂—C₁₋₆-alkyl, —N(R13)R14, —(C₁₋₅-alkylene)-N(R13)R14, —S(═O)—NH₂, —S(═O)₂—NH—C₁₋₆-alkyl, —S(═O)₂NH-phenyl, —NHS(═O)₂—C₁₋₆-alkyl, cyclopropyl, cyclobutyl, cyclopentyl, cyclohexyl, pyrrolidinyl, piperidinyl, phenyl, thiophenyl and benzyl; wherein in each case the cyclic moieties cyclopropyl, cyclobutyl, cyclopentyl, cyclohexyl, pyrrolidinyl, piperidinyl, phenyl, thiophenyl and benzyl can optionally be substituted with 1, 2, 3, 4 or 5 substituent(s) independently selected from the group consisting of F, Cl, Br, I, —OH, —CF₃, —CN, —NO₂, —C₁₋₆-alkyl, —O—C₁₋₆-alkyl, —OCF₃ and —SCF₃ and whereby R9, R10, R13and R14, independently of one another, represent —H or —C₁₋₆-alkyl or R9 and R10, in each case together with the nitrogen atom to which they are bound, form a radical selected from the group consisting of pyrrolidinyl, imidazolidinyl, piperazinyl, piperidinyl, thiomorpholinyl, morpholinyl, azepanyl and diazepanyl which may be at least mono-substituted with one or more identical or different C₁₋₆ alkyl radicals and whereby R11 and R12, independently of one another, represent —H, —C₁₋₆alkyl, —C(═O)O-C₁₋₆-alkyl, C₃₋₈-cycloalkyl, —C₁₋₅-alkylene-C₃₋₈-cycloalkyl, —C₁₋₆-alkylene-O—C₁₋₆-alkyl or —C₁₋₆-alkyl substituted with one or more hydroxy groups or R11 and R12 in each case together with the bridging nitrogen atom form a radical selected from the group consisting of pyrrolidinyl, imidazolidinyl, piperazinyl, piperidinyl, thiomorpholinyl, morpholinyl, azepanyl and diazepanyl which may be at least mono-substituted with one or more substituents independently selected from the group consisting —C₁₋₆-alkyl, —C(═O)—C₁₋₆-alkyl, —C(═O)—O—C₁₋₆-alkyl, —C(═O)—NH—C₁₋₆-alkyl, —C(═S)—NH—C₁₋₆-alkyl, oxo (═O), —C₁₋₆-alkyl substituted with one or more hydroxy groups, —(C₁₋₆-alkylene)-O—C₁₋₆-alkyl and —C(═O)—NH₂.

In other embodiments, said mono- or polycyclic ring system may optionally be substituted with 1, 2, 3, 4 or 5 substituent(s) independently selected from the group consisting of oxo (═O), thioxo (═S), —CF₃, —C₂F₅, —C₃F₂, —C₄F₉, —CH₂Cl, —CHCl₂, —C₂H₄Cl, methyl, ethyl, n-propyl, isopropyl, n-butyl, isobutyl, 2-butyl, tert-butyl, n-pentyl, 2-pentyl, n-hexyl, —CH₂—OH, —CH₂—CH₂—OH, —CH₂—CH₂—CH₂—OH, —O—CH₂—O—CH₃, —O—CH₂—CH₂—O—CH₃, —O—CH₂—O—C₂H₅, —C(OCH₃)(C₂H₅)₂, —C(OCH₃)(CH₃)₂, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —C(═O)—OH, —C(═O)—O—CH₃, —C(═O)—O—C₂H₅, —C(═O)—O—C₃H₇, —C(═O)—O—C(CH₃)₃, —O—C(═O)—CH₃, —O—C(═O)—C₂H₅, —O—C(═O)—CH(CH₃)₂, —O—C(═O)—CH₂—CH₂—CH₃, —O—C(═O)—C(CH₃)₃, F, Cl, Br, I, —CN, —OCF₃, —O—C₂F₅, —O—C₃F₂, —O—C₄F₉, —SCF₃, —SCF₂H, —SCFH₂, —OH, —SH, —SO₃H, —NH—C(═O)—CH₃, —NH—C(═O)—C₂H₅, —NH—C(═O)—C(CH₃)₃, —NO₂, —CHO, —C(═O)—CH₃, —C(═O)—C₂H₅, —C(═O)—C(CH₃)₃, —C(═O)—CF₃, —C(═O)—C₂F₅, —C(═O)—C₃F₂, —C(═S)—NH—CH₃, —C(═S)—NH—C₂H₅, —CF₂H, —CFH₂, —C(═O)—NH₂, —C(═O)—NH—CH₃, —C(═O)—NH—C₂H₅, —C(═O)—NH—C₃H₂, —C(═O)—N(CH₃)₂, —C(═O)—N(C₂H₅)₂, —C(═O)—NH—NH—CH₃, —C(═O)—NH—NH—C₂H₅, —C(═O)—NH—NH₂, —C(═O)—NH—N(CH₃)₂, —S(═O)—CH₃, —S(═O)—C₂H₅, —S(═O)—C₃H₇, —S(═O)₂—CH₃, —S(═O)₂—C₂H₅, —S(═O)—C₃H₇, —S(═O)₂-phenyl, —NH₂, —NH—CH₃, —NH—C₂H₅, —N(CH₃)₂, —N(C₂H₅)₂, —CH₂—N(CH₃)₂, —(CH₂)-morpholinyl, —(CH₂)-piperidinyl, —(CH₂)-piperazinyl, —(CH₂)—N(C₂H₅)₂, —CH₂—N(C₃H₇)₂, —CH₂—N(C₄H₉)₂, —CH₂—N(CH₃)(C₂H₅), —S(═O)—NH₂, —S(═O)₂—NH—CH₃, —S(═O)₂—NH-phenyl, —NH—S(═O)₂—CH₃, cyclopropyl, cyclobutyl, cyclopentyl, cyclohexyl, pyrrolidinyl, piperidinyl, phenyl, thiophenyl and benzyl, whereby said thiophenyl radical can be substituted with 1, 2 or 3 substituents independently selected from the group consisting of F, Cl, Br, methyl, ethyl and n-propyl.

If one or more of the residues of R1 represent or comprise an aliphatic radical, C₁₋₁₆ alkyl radical, C₂₋₁₆ alkenyl radical or C₂₋₁₆ alkynyl radical, unless defined otherwise, may optionally be substituted with 1, 2, 3, 4, 5, 6, 7, 8 or 9 substituents independently selected from the group consisting of —OH, —SH, F, Cl, Br, I, —O—C₁₋₆-alkyl, —OCF₃, —O—C₂F₅, —O—C₃F₇, —O—C₄F₉, —O-phenyl, —O-benzyl, —S—C₁₋₆-alkyl, —S—phenyl, —S-benzyl, —CF₃, —C₂F₅, —C₃F₂, —C₄F₉, —NH₂, —NH—C₁₋₆-alkyl, —(C₁₋₆-alkyl)₂, —NH-phenyl, —NH-benzyl, —N(C₁₋₆-alkyl)-phenyl, —N(C₁₋₆-alkyl)-benzyl, —N⁺(C₁₋₆-alkyl)₃, —C(═O)—OH, —C(═O)—O—C₁₋₆-alkyl, —C(═O)—O-benzyl, —C(═O)—O-phenyl, —C(═O)—NH₂, —C(═O)—NH—C₁₋₆-alkyl, —C(═O)—N(C₁₋₆-alkyl)₂, —C(═O)—NH-phenyl, —C(═O)—NH-benzyl, —CN, —NO₂, —S(═O)—NH₂, —CHO, —C(═O)—C₁₋₆-alkyl, —O—C(═O)—C₁₋₆-alkyl, —C(═O)-benzyl, —C(═O)-phenyl, —S(═O)—C₁₋₆-alkyl, —S(═O)₂-C₁₋₆-alkyl, —S(═O)-phenyl, —S(═O)₂-phenyl, —S(═O)-benzyl, —S(═O)-phenyl, —O—S(═O)—C₁₋₆-alkyl-, —O—S(═O)-phenyl, —O—S(═O)-benzyl, —O—S(═O)₂-C₁₋₆-alkyl-, —O—S(═O)₂-phenyl, —O—S(═O)₂-benzyl, —NH—S(═O)—C₁₋₆-alkyl, —NH—C(═O)—O—C₁₋₆-alkyl and —NH—C(═O)—C₁₋₆-alkyl; whereby in each case the cyclic moieties phenyl and benzyl can optionally be substituted with 1, 2, 3, 4 or 5 substituent(s) independently selected from the group consisting of F, Cl, Br, I, —OH, —C(═O)—OH, —C(═O)—O—C₁₋₆-alkyl, —C(═O)—H, —C(═O)—C₁₋₆-alkyl, —CF₃, —CN, —NO₂, —C₁₋₆-alkyl, —O—C₁₋₆-alkyl, —O—CF₃ and —S—CF₃.

In other embodiments, aliphatic radicals, C₁₋₁₆ alkyl radicals, C₂₋₁₆ alkenyl radical and C₂₋₁₆ alkynyl radicals may optionally be substituted with 1, 2, 3, 4, 5, 6, 7, 8 or 9 substituents independently selected from the group consisting of —OH, F, Cl, Br, I, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C2H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —CN, —NO₂, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —NH—C(═O)—CH₃, —NH—C(═O)—C₂H₅, —NH—C(═O)—C(CH₃)₃, —NH—C(═O)—O—CH₃, —NH—C(═O)—O—C₂H₅, —NH—C(═O)—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃.

Suitable alkyl radicals, preferably C₁₋₁₆ alkyl radicals, are selected from the group consisting of methyl, ethyl, n-propyl, isopropyl, n-butyl, isobutyl, sec-butyl, tert-butyl, n-pentyl, 2-pentyl, 3-pentyl, neo-pentyl, n-hexyl, 2-hexyl, 3-hexyl, n-heptyl, 2-heptyl, 3-heptyl, 4-heptyl, n-octyl, 2-octyl, 3-octyl, 4-octyl, 2-(6-methyl)-heptyl, 2-(5-methyl)-heptyl, 2-(5-methyl)-hexyl, 2-(4-methyl)-hexyl, 2-(7-methyl)-octyl, 2-(6-methyl)-octyl, n-nonyl, n-decyl, n-undecyl, n-dodecyl, n-tridecyl, n-tetradecyl, n-pentadecyl and n-hexadecyl.

Suitable at least mono-substituted alkyl radicals are selected from the group consisting of —CF₃, —CH₂F, —CF₂H, —CH₂—O—CH₃, —C₂F₅, —CH₂—CH₂—F, —CH₂—CN, —CH₂—OH, —CH₂—CH₂—CN, —CH₂—CH₂—OH, —CH₂—CH₂—OCH₃, —CH₂—CH₂—CH₂—CN, —CH₂—CH₂—CH₂—OH, —CH₂—CH₂—CH₂—O—CH₃, —CH₂—CH₂—CH₂—CH₂—O—CH₃, —CH₂—NH₂, —CH₂—N(CH₃)₂, —CH₂N(C₂H₅)₂, —CH₂—CH—NH₂, —CH₂—CH₂—N(CH₃)₂, —CH₂—CH₂—N(C₂H₅)₂, —CH₂—CH₂—CH₂—NH₂, —CH₂—CH₂—CH₂—N(CH₃)₂ and —CH₂—CH₂—CH₂—N(C₂H₅)₂.

An alkenyl radical according to the present invention comprises at least one carbon-carbon double bond. Suitable alkenyl radicals, preferably C₂₋₁₆ alkenyl radicals, are selected from the group consisting of vinyl, n-propenyl, n-butenyl, n-pentenyl, n-hexenyl, n-heptenyl, n-octenyl, n-nonenyl, n-decenyl, n-undecenyl, n-dodecenyl, n-tridecenyl, n-tetradecenyl, n-pentadecenyl and n-hexadecenyl.

An alkynyl radical comprises at least one carbon-carbon triple bond. Suitable alkynyl radicals, preferably C246 alkynyl radicals, are selected from the group consisting of ethynyl, propynyl, n-butynyl, n-pentynyl, n-hexynyl, n-octynyl, n-nonynyl, n-decynyl, n-undecynyl, n-dodecynyl, n-tridecynyl, n-tetradecynyl, n-pentadecynyl and n-hexadecynyl.

If any of the substituents represents an alkylene group, an alkenylene group or an alkynylene group, which may be substituted, said alkylene group, alkenylene group or alkynylene group may-if not defined otherwise-be unsubstituted or substituted by one or more substituents, preferably unsubstituted or substituted with 1, 2 or 3 substituent(s). Said substituent(s) may preferably be selected independently from the group consisting of —O—C₁₋₆-alkyl, —S—C₁₋₆-alkyl, —F, —Cl, —Br, —I, —CN, —CF₃, —OCF₃, —SCF₃, —OH, —SH, —SO₃H, —NH₂, —NH(C₁₋₆-alkyl), —N(C₁₋₆-alkyl)₂ and phenyl. More preferably said substituent(s) may be selected from the group consisting of —F, Cl, Br, I, —CN, —CF₃, —OCF₃, —SCF₃, —OH, —SH, —SO₃H, —NH₂, —NH—CH₃, —N(CH₃)₂, —O—CH₃ and —O—C₂H₅. An alkenylene group comprises at least one carbon-carbon double bond, an alkynylene group comprises at least one carbon-carbon triple bond.

Suitable alkylene groups, preferably C₁₋₅-alkylene groups, include —(CH₂)—, —CH(CH₃)—, —CH(phenyl), —(CH₂)₂—, —(CH₂)₃—, —(CH₂)₄—, —(CH₂)₅— and —(CH₂)₆—, suitable alkenylene groups, preferably C₂₋₅-alkenylene groups, include —CH═CH—, —CH₂—CH═CH— and —CH═CH—CH₂— and suitable alkynylene groups, preferably C₂₋₅-alkynylene groups, include —C≡C—, —CH₂—C≡C— and —C≡C—CH₂—.

In other embodiments, R1 represents —C(═O)OR7, —C(═O)OH, —P(═O)(OR7)₂, —P(═O)(OH)₂, P(═O)(OH)(OR7), —C(═O)NH₂, —C(═O)NHR7, —C(═O)N(R7)R7, —C(═O)H, —C(═O)R7, —S(═O)R7, —(CHR8)ₘ—OC(═O)R7, —(CHR8)ₘ—OC(═O)OR7, —(CHR8)ₘ—OP(═O)(OR7)₂, —(CHR8)ₘ—OC(═O)NHR7, or —(CHR8)ₘ—OC(═O)N(R7)R8, —S(═O)₂—R7, —C(═S)OR7 or —C(═S)R7; wherein m is independently 1, 2 or 3; wherein R7, independently of one another, in each case represent an unsubstituted or at least mono-substituted C₁₋₁₆ alkyl radical, C₂₋₁₆ alkenyl radical or C₂₋₁₆ alkynyl radical; or an unsubstituted or at least mono-substituted 6- or 10-membered aryl radical, which may be condensed with an unsubstituted or at least mono-substituted mono- or polycyclic ring system and/or may be bonded via an unsubstituted or at least mono-substituted C₁₋₅ alkylene group, C₂₋₅ alkenylene group or C₂₋₅ alkynylene group; or an unsubstituted or at least mono-substituted 5- to 14-membered heteroaryl radical, which may be condensed with an unsubstituted or at least mono-substituted mono- or polycyclic ring system and/or may be bonded via an unsubstituted or at least mono-substituted C₁₋₅ alkylene group, C₂₋₅ alkenylene group or C₂₋₅ alkynylene group; and wherein R8 represents —H or a saturated or unsaturated, unsubstituted or at least mono-substituted aliphatic radical; whereby the rings of the aforementioned ring system are in each case independently of one another 5- 6- or 7-membered and may in each case independently of one another optionally contain 1, 2 or 3 heteroatom(s) independently selected from the group consisting of nitrogen, oxygen and sulfur; the aforementioned heteroaryl radicals in each case optionally contain 1, 2, 3 or 4 heteroatom(s) independently selected from the group consisting of nitrogen, oxygen and sulfur as ring member(s); the aforementioned heterocycloalkyl radicals and heterocycloalkenyl radicals in each case optionally contain 1, 2, 3 or 4 heteroatom(s) independently selected from the group consisting of nitrogen, oxygen and sulfur as ring member(s).

In other embodiments, R1 represents —C(═O)R7, —OC(═O)—(CHR8)ᵣ—R7, —OC(═O)OR7, —C(═O)OR7, —C(═O)OH, —P(═O)(OR7)₂, P(═O)(OR7)(OH), —P(═O)(OH)₂, —C(═O)NH₂, —C(═O)NHR7, —C(═O)—N(R7)R7, —C(═O)H, —C(═O)R7, —S(═O)R7, —(CHR8)ᵣ—OC(═O)R7, —(CHR8)ₘ—O—C(═O)—OR7, —(CHR8)ₘ—OP(═O)(OR7)₂, —(CHR8)ₘ—OC(═O)NHR7, —(CHR8)ₘ—OC(═O)N(R7)R7, —S(═O)₂R7, —C(═S)OR7 or —C(═S)R7; wherein r is 1, 2, 3, 4, 5, 6, 7 or 8; wherein m is 1, 2 or 3; wherein R7, independently of one another, in each case represent a radical selected from the group consisting of —H, methyl, ethyl, n-propyl, isopropyl, n-butyl, isobutyl, sec-butyl, tert-butyl, n-pentyl, 2-pentyl, 3-pentyl, neo-pentyl, n-hexyl, 2-hexyl, 3-hexyl, n-heptyl, 2-heptyl, 3-heptyl, 4-heptyl, n-octyl, 2-octyl, 3-octyl, 4-octyl, vinyl, n-propenyl, n-butenyl, n-pentenyl, n-hexenyl, ethynyl, propynyl, n-butynyl, n-pentynyl and n-hexynyl, which may optionally be substituted with 1, 2, 3, 4, 5, 6, 7, 8 or 9 substituent(s) independently selected from the group consisting of —OH, —SH, —F, —Cl, —Br, —I, —O—C₁₋₆-alkyl, —OCF₃, —O—C₂F₅, —O—C₃F₂, —O—C₄F₉, —O-phenyl, —O-benzyl, —S—C₁₋₆-alkyl, —S-phenyl, —S-benzyl, —CF₃, —C₂F₅, —C₃F₂, —C₄F₉, —NH₂, —NH—C₁₋₆-alkyl, —N(C₁₋₆-alkyl)₂, —NH-phenyl, —NH-benzyl, —N(C₁₋₆-alkyl)-phenyl, —N(C₁₋₆-alkyl)-benzyl, —N⁺(C₁₋₆-alkyl)₃, —C(═O)—OH, —C(═O)—O—C₁₋₆-alkyl, —C(═O)—O-benzyl, —C(═O)—O-phenyl, —C(═O)—NH₂, —C(═O)—NH—C₁₋₆-alkyl, —C(═O)—N(C₁₋₆-alkyl)₂, —C(═O)—NH-phenyl, —C(═O)—NH-benzyl, —CN, —NO₂, —S(═O)—NH₂, —CHO, —C(═O)—C₁₋₆-alkyl, —C(═O)-benzyl, —C(═O)-phenyl, —O—C(═O)—C₁₋₆-alkyl, —S(═O)—C₁₋₆-alkyl, —S(═O)₂—C₁₋₆-alkyl, —S(═O)-phenyl, —S(═O)₂phenyl, —S(═O)-benzyl, —S(═O)-phenyl, —O—S(═O)—C₁₋₆-alkyl-, —O—S(═O)-phenyl, —O—S(═O)-benzyl, —O—S(═O)₂—C₁₋₆-alkyl-, —O—S(═O)₂-phenyl, —O—S(═O)₂-benzyl, —NH—S(═O)—C₁₋₆-alkyl, —NH—C(═O)—O—C₁₋₆-alkyl and —NH—C(═O)—C₁₋₆-alkyl; whereby in each case the cyclic moieties phenyl and benzyl can optionally be substituted with 1, 2, 3, 4 or 5 substituent(s) independently selected from the group consisting of F, CI, Br, I, —OH, —C(═O)—OH, —C(═O)—O—C₁₋₆-alkyl, —C(═O)—H, —C(═O)—C₁₋₆-alkyl, —CF₃, —CN, —NO₂, —C₁₋₆-alkyl, —O—C₁₋₆-alkyl, —O—CF₃ and —S—CF₃. or a radical selected from the group consisting of phenyl, naphthyl, pyridinyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, indolyl and isoindolyl, which may be bonded via a —(CH₂)—, —(CH₂)—(CH₂)—, —(CH₂)—(CH₂)—(CH₂)— or —CH═CH-group and/or may optionally be substituted with 1, 2, 3, 4 or 5 substituent(s) independently selected from the group consisting of —CF₃, methyl, ethyl, n-propyl, isopropyl, n-butyl, isobutyl, 2-butyl, tert-butyl, n-pentyl, 2-pentyl, n-hexyl, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S-CH₂—CH₂—CH₂—CH₃, —S—C(CH₃)₃, F, Cl, Br, I, —CN, —OCF₃, —SCF₃, —SCF₂H, —SCFH₂, —OH, —SH, —NO₂, —CHO, —C(═O)—CH₃, —C(═O)—C₂H₅, —C(═O)—C(CH₃)₃, —CF₂H, —CFH₂, —C(═O)—NH₂, —C(═O)—NH—CH₃, —C(═O)—NH—C₂H₅, —C(═O)—NH—C₃H₇, —C(═O)—N(CH₃)₂, —C(═O)—N(C₂H₅)₂, —S(═O)—CH₃, —S(═O)—C₂H₅, —S(═O)—C₃H₇, —S(═O)₂—CH₃, —S(═O)₂—C₂H₅, —S(═O)₂—C₃H₇, —NH₂, —NH—CH₃, —NH—C₂H₅, —N(CH₃)₂, —N(C₂H₅)₂, —O—C(═O)—CH₃, —O—C(═O)—C₂H₅, —O—C(═O)—CH(CH₃)₂, —O—C(═O)—CH₂—CH₂—CH₃, —CH₂—N(CH₃)₂, —(CH₂)—N(C₂H₅)₂, —CH₂—N(C₃H₇)₂, —CH₂—N(C₄H₉)₂, —CH₂—N(CH₃)(C₂H₅) and —(CH₂)-morpholinyl; and wherein R8 represents a radical selected from the group consisting of —H, methyl, ethyl, n-propyl, isopropyl, n-butyl, isobutyl, sec-butyl, tert-butyl, n-pentyl, 2-pentyl, 3-pentyl, neo-pentyl, n-hexyl, 2-hexyl, 3-hexyl, n-heptyl, 2-heptyl, 3-heptyl, 4-heptyl, n-octyl, 2-octyl, 3-octyl, 4-octyl, vinyl, n-propenyl, n-butenyl, n-pentenyl, n-hexenyl, ethynyl, propynyl, n-butynyl, n-pentynyl and n-hexynyl, which may optionally be substituted with 1, 2, 3, 4, 5, 6, 7, 8 or 9 substituent(s) independently selected from the group consisting of —OH, —SH, F, Cl, Br, I, —O—C₁₋₆-alkyl, —OCF₃, —O—C₂F₅, —O—C₃F₇, —O—C₄F₉, —O-phenyl, —O-benzyl, —S—C₁₋₆-alkyl, —S-phenyl, —S-benzyl, —CF₃, —C₂F₅, —C₃F₇, —C₄F₉, —NH₂, —NH—C₁₋₆-alkyl, —N(C₁₋₆-alkyl)₂, —NH-phenyl, —NH-benzyl, —N(C₁₋₆-alkyl)-phenyl, —N(C₁₋₆-alkyl)-benzyl, —N⁺(C₁₋₆-alkyl)₃, —C(═O)—OH, —C(═O)—O—C₁₋₆-alkyl, —C(═O)—O-benzyl, —C(═O)—O-phenyl, —C(═O)—NH₂, —O—C(═O)—C₁₋₆-alkyl, —C(═O)—NH—C₁₋₆-alkyl, —C(═O)—N(C₁₋₆-alkyl)₂, —C(═O)—NH-phenyl, —C(═O)—NH-benzyl, —CN, —NO₂, —S(═O)—NH₂, —CHO, —C(═O)—C₁₋₆-alkyl, —C(═O)-benzyl, —C(═O)-phenyl, —S(═O)—C₁₋₆-alkyl, —S(═O)₂—C₁₋₆-alkyl, —S(═O)-phenyl, —S(═O)₂-phenyl, —S(═O)-benzyl, —S(═O)-phenyl, —O—S(═O)—C₁₋₆-alkyl-, —O—S(═O)-phenyl, —O—S(═O)-benzyl, —O—S(═O)₂—C₁₋₆-alkyl-, —O—S(═O)₂-phenyl, —O—S(═O)₂-benzyl, —NH—S(═O)—C₁₋₆-alkyl, —NH—C(═O)—O—C₁₋₆alkyl and —NH—C(═O)—C₁₋₆-alkyl; wherein in each case the cyclic moieties phenyl and benzyl can optionally be substituted with 1, 2, 3, 4 or 5 substituent(s) independently selected from the group consisting of F, Cl, Br, I, —OH, —C(═O)—OH, —C(═O)—O—C₁₋₆-alkyl, —C(═O)—H, —C(═O)—C₁₋₆-alkyl, —CF₃, —CN, —NO₂, —C₁₋₆-alkyl, —O—C₁₋₆-alkyl, —O—CF₃ and —S—CF₃; optionally in form of one of its stereoisomers, preferably enantiomers or diastereomers, a racemate or in form of a mixture of at least two of its stereoisomers, preferably enantiomers and/or diastereomers, in any mixing ratio, or a corresponding N-oxide thereof, or a physiologically acceptable salt thereof, or a corresponding solvate thereof.

In other embodiments, R1 is selected from: —C(═O)OR7, —C(═O)OH, —P(═O)(OR7)₂, P(═O)(OR7)(OH), —P(═O)(OH)₂, —C(═O)NH₂, —C(═O)NHR7, —C(═O)N(R7)R7, —C(═O)H, —C(═O)R7, —S(═O)R7, —(CHR8)ₘ—OC(═O)R7, —(CHR8)ₘ—OC(═O)OR7, —(CHR8)ₘ—OP(═O)(OR7)₂, —(CHR8)ₘ—OC(═O)NHR7, —(CHR8)ₘ—OC(═O)N(R7)R7, —S(═O)₂R7, —C(═S)OR7 or —C(═S)R7; wherein m is 1, 2 or 3; wherein R7, independently of one another, in each case represent a radical selected from the group consisting of methyl, ethyl, n-propyl, isopropyl, n-butyl, isobutyl, sec-butyl, tert-butyl, n-pentyl, 2-pentyl, 3-pentyl, neo-pentyl, n-hexyl, 2-hexyl, 3-hexyl, n-heptyl, 2-heptyl, 3-heptyl, 4-heptyl, n-octyl, 2-octyl, 3-octyl, 4-octyl, vinyl, n-propenyl, n-butenyl, n-pentenyl, n-hexenyl, ethynyl, propynyl, n-butynyl, n-pentynyl and n-hexynyl, which may optionally be substituted with 1, 2, 3, 4, 5, 6, 7, 8 or 9 substituent(s) independently selected from the group consisting of —OH, F, Cl, Br, I, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺C(CH₃H₅)₃, —N⁺[C(CH₃)₃]₃, —CN, —NO₂, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —O—C(═O)—CH₃, —O—C(═O)—C₂H₅, —O—C(═O)—CH(CH₃)₂, —O—C(═O)—CH₂—CH₂—CH₃, —NH—C(═O)—CH₃, —NH—C(═O)—C₂H₅, —NH—C(═O)—C(CH₃)₃, —NH—C(═O)—O—CH₃, —NH—C(═O)—O—C₂H₅, —NH—C(═O)—O—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃; or a radical selected from the group consisting of phenyl, naphthyl, pyridinyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, indolyl and isoindolyl, which may be bonded via a —(CH₂)—, —(CH₂)—(CH₂)—, —(CH₂)—(CH₂)—(CH₂)— or —CH═CH-group and/or may optionally be substituted with 1, 2, 3, 4 or 5 substituent(s) independently selected from the group consisting of —CF₃, methyl, ethyl, n-propyl, isopropyl, n-butyl, isobutyl, 2-butyl, tert-butyl, n-pentyl, 2-pentyl, n-hexyl, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, F, Cl, Br, I, —CN, —OCF₃, —SCF₃, —SCF₂H, —SCFH₂, —OH, —SH, —NO₂, —CHO, —C(═O)—CH₃, —C(═O)—C₂H₅, —C(═O)—C(CH₃)₃, —CF₂H, —CFH₂, —C(═O)—NH₂, —C(═O)—NH—CH₃, —C(═O)—NH—C₂H₅, —C(═O)—NH—C₃H₇, —C(═O)—N(CH₃)₂, —C(═O)—N(C₂H₅)₂, —S(═O)—CH₃, —S(═O)—C₂H₅, —S(═O)—C₃H₇, —S(═O)₂—CH₃, —S(═O)₂—C₂H₅, —S(═O)₂—C₃H₇, —NH₂, —NH—CH₃, —NH—C₂H₅, —N(CH₃)₂, —N(C₂H₅)₂, —O—C(═O)—CH₃, —O—C(═O)—C₂H₅, —O—C(═O)—CH(CH₃)₂, —O—C(═O)—CH₂—CH₂—CH₃, —CH₂—N(CH₃)₂, —(CH₂)—N(C₂H₅)₂, —CH₂—N(C₃H₂)₂, —CH₂—N(C₄H₉)₂, —CH₂—N(CH₃)(C₂H₅) and —(CH₂)-morpholinyl; and wherein R8 represent a radical selected from the group consisting of methyl, ethyl, n-propyl, isopropyl, n-butyl, isobutyl, sec-butyl, tert-butyl, n-pentyl, 2-pentyl, 3-pentyl, neo-pentyl, n-hexyl, 2-hexyl, 3-hexyl, n-heptyl, 2-heptyl, 3-heptyl, 4-heptyl, n-octyl, 2-octyl, 3-octyl, 4-octyl, vinyl, n-propenyl, n-butenyl, n-pentenyl, n-hexenyl, ethynyl, propynyl, n-butynyl, n-pentynyl and n-hexynyl, which may optionally be substituted with 1, 2, 3, 4, 5, 6, 7, 8 or 9 substituent(s) independently selected from the group consisting of —OH, F, Cl, Br, I, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S —C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —CN, —NO₂, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—C(═O)—CH₃, —O—C(═O)—C₂H₅, —O—C(═O)—CH(CH₃)₂, —O—C(═O)—CH₂—CH₂—CH₃, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —NH—C(═O)—CH₃, —NH—C(═O)—C₂H₅, —NH—C(═O)—C(CH₃)₃, —NH—C(═O)—O—CH₃, —NH—C(═O)—O—C₂H₅, —NH—C(═O)—O—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃; optionally in form of one of its stereoisomers, preferably enantiomers or diastereomers, a racemate or in form of a mixture of at least two of its stereoisomers, preferably enantiomers and/or diastereomers, in any mixing ratio, or a corresponding N-oxide thereof, or a physiologically acceptable salt thereof, or a corresponding solvate thereof.

In other embodiments, R1 is selected from: —C(═O)OR7, —C(═O)OH, —P(═O)(OR7)₂, P(═O)(OR7)(OH), —P(═O)(OH)₂, —P(═O)(OH)(OR16), —P(═O)(OR16)(OR17), —C(═O)NH₂, —C(═O)—NHR7, —C(═O)N(R7)R7, —C(═O)H, —C(═O)R7, —S(═O)R7, —(CHR8)ₘ—OC(═O)R7, —(CHR8)ₘ—OC(═O)OR7, —(CHR8)ₘ—OP(═O)(OR8)₂, —(CHR8)ₘ—OC(═O)NHR7, —(CHR8)ₘ—OC(═O)N(R7)R7, —S(═O)₂ R7, —C(═S)OR7 or —C(═S)R7; wherein m is 1, 2 or 3;
wherein R7, independently of one another, in each case represent a radical selected from the group consisting of methyl, ethyl, n-propyl, isopropyl, n-butyl, isobutyl, sec-butyl, tert-butyl, n-pentyl, 2-pentyl, 3-pentyl, neo-pentyl and n-hexyl, which may optionally be substituted with 1, 2, 3, 4, 5, 6, 7, 8 or 9 substituent(s) independently selected from the group consisting of —OH, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —NH—C(═O)—CH₃, —O—C(═O)—CH₃, —O—C(═O)—C₂H₅, —O—C(═O)—CH(CH₃)₂, —O—C(═O)—CH₂—CH₂—CH₃, —NH—C(═O)—C₂H₅, —NH—C(═O)—C(CH₃)₃, —NH—C(═O)—O—CH₃, —NH—C(═O)—O—C₂H₅, —NH—C(═O)—O—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃; and
wherein R8 represents a radical selected from the group consisting of methyl, ethyl, n-propyl, isopropyl, n-butyl, isobutyl, sec-butyl, tert-butyl, n-pentyl, 2-pentyl, 3-pentyl, neo-pentyl and n-hexyl, which may optionally be substituted with 1, 2, 3, 4, 5, 6, 7, 8 or 9 substituent(s) independently selected from the group consisting of —OH, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —NH—C(═O)—CH₃, —NH—C(═O)C₂H₅, —NH—C(═O)—C(Ch₃)₃, —NH—C(50 O)—O—CH₃, —NH—C(50 O)—O—C₂H₅, —NH—C(═O)—O—C(CH₃)₃, —C(═O)—NH₂, —C(═O)—NH—CH₃, —C(═O)—N H—C₂H₅, —C(═O)—NH—C(CH₃)₃, —O—C(═O)—CH₃, —O—C(═O)—C₂H₅, —O—C(═O)—CH(CH₃)₂, —O—C(═O)—CH₂—CH₂—CH₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃;
wherein R16 and R17 are independently selected from salt-forming cations, including alkali metals (e.g., sodium and potassium); alkaline earth metals (e.g., calcium and magnesium); and cations of pharmaceutically acceptable organic bases (e.g., quaternized or protonated amines, including alkylamines, hydroxyalkylamines, monoamines, diamines and naturally occurring amines). In one embodiment, the pharmaceutically acceptable organic base is selected from choline, betaine, caffeine, N,N′-dibenzylethylenediamine, diethylamine, 2-diethylaminoethanol, 2-dimethylaminoethanol, ethanolamine, ethylenediamine. N-ethylmorpholine, N-ethylpiperidine, glucamine, hydrabamine, isopropylamine, methylglucamine, morpholine, piperidine, polyamine resins, procaine, purines, theobromine, triethylamine, trimethylamine, tripropylamine, tromethamine, tetramethylammonium hydroxide, benzyltrimethylammonium hydroxide, tris(hydroxymethyl)aminomethane (TRIS), N-(2-hydroxyethyl)pyrrolidine, piperazine, glucosamine, arginine, lysine or histidine.

In other embodiments, R1 is selected from: —C(═O)OR7, —C(═O)OH, —P(═O)(OR7)₂, P(═O)(OR7)(OH), —P(═O)(OH)₂, —C(═O)NH₂, —C(═O)NHR7, —C(═O)N(R7)R7, —C(═O)H, —C(═O)R7, —S(═O)R7, —(CHR8)ₘ—OC(═O)R7, —(CHR8)ₘ—OC(═O)OR7, (CHR8)ₘ—OP(═O)(OR7)₂, —(CHR8)ₘ—OC(═O)—NHR7, —(CHR8)ₘ—OC(═O)N(R7)R7, —S(═O)₂—R7, —C(═S)OR7, or —C(═S)R7; wherein m is 1, 2 or 3;
wherein R7 is independently of one another, in each case represent a radical selected from the group consisting of methyl, ethyl, n-propyl, isopropyl, n-butyl, isobutyl, sec-butyl, tert-butyl, n-pentyl, 2-pentyl, 3-pentyl, neo-pentyl and n-hexyl, which may optionally be substituted with 1, 2, 3, 4, 5, 6, 7, 8 or 9 substituent(s) independently selected from the group consisting of —OH, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —O—C(═O)—CH₃, —O—C(═O)—C₂H₅, —O—C(═O)—CH(CH₃)₂, —O—C(═O)—CH₂—CH₂—CH₃, —NH—C(═O)—CH₃, —NH—C(═O)—C₂H₅, —NH—C(═O)—C(CH₃)₃, —NH—C(═O)—O—CH₃, —NH—C(═O)—O—C₂H₅, —NH—C(═O)—O—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —C(═O)—O—C₂H₅, —C(═O)—O—C(CH₃)₃, —C(═O)—CH₃, —C(═O)—C₂H₅ and —C(═O)—C(CH₃)₃; and
wherein R8 represent a radical selected from the group consisting of methyl, ethyl, n-propyl, isopropyl, n-butyl, isobutyl, sec-butyl, tert-butyl, n-pentyl, 2-pentyl, 3-pentyl, neo-pentyl and n-hexyl, which may optionally be substituted with 1, 2, 3, 4, 5, 6, 7, 8 or 9 substituent(s) independently selected from the group consisting of —OH, —O—CH₃, —O—C₂H₅, —O—CH₂—CH₂—CH₃, —O—CH(CH₃)₂, —O—CH₂—CH₂—CH₂—CH₃, —O—C(CH₃)₃, —S—CH₃, —S—C₂H₅, —S—CH₂—CH₂—CH₃, —S—CH(CH₃)₂, —S—CH₂—CH₂—CH₂—CH₃, —S—C(CH₃)₃, —NH₂, —NH—CH₃, —NH—C₂H₅, —N(CH₃)₂, —N(C₂H₅)₂, —N⁺(CH₃)₃, —N⁺(C₂H₅)₃, —N⁺[C(CH₃)₃]₃, —S(═O)—CH₃, —S(═O)—C₂H₅, —S(═O)—C₃H₇, —S(═O)—C(CH₃)₃, —S(═O)₂—CH₃, —S(═O)₂—C₂H₅, —S(═O)₂—C₃H₇, —S(═O)₂—C(CH₃)₃, —O—S(═O)—CH₃, —O—S(═O)—C₂H₅, —O—S(═O)—C₃H₇, —O—S(═O)—C(CH₃)₃, —O—S(═O)₂—CH₃, —O—S(═O)₂—C₂H₅, —O—S(═O)₂—C₃H₇, —O—S(═O)₂—C(CH₃)₃, —NH—C(═O)—CH₃, —NH—C(═O)—C₂H₅, —NH—C(═O)—C(CH₃)₃, —NH—C(═O)—O—CH₃, —NH—C(═O)—O—C₂H₅, —NH—C(═O)—O—C(CH₃)₃, —C(═O)—NH₂, —C(═O)—NH—CH₃, —C(═O)—NH—C₂H₅, —C(═O)—NH—C(CH₃)₃, —C(═O)—N(CH₃)₂, —C(═O)—N(C₂H₅)₂, —C(═O)—OH, —C(═O)—O—CH₃, —O—C(═O)—CH₃, —O—C(═O)—C₂H₅, —O—C(═O)—CH(CH₃)₂, —O—C(═O)—CH₂—CH₂—CH₃, —O—C(═O)—O—C₂H₅, —C(═O)—O—C(CH₃)₃, —C(═O)—CH₃, —C(═O)—C₂H₅ and —C(═O) C(CH₃)₃.

In one embodiment, R1 and the xanthophyll oxygen to which it is bound, together form a beta-D-glucopyranosyl, 3-O-beta-D-glucopyranosyl-beta-D-glucopyranosyl, or beta-D-glucopyranosyl-(1-3)-beta-D-glucopyranosyl-(1-3)-D-glucopyranosyl; each optionally substituted with 1, 2 or 3 beta-D-glucopyranosyl or 3-O-beta-D-glucopyranosyl-beta-D-glucopyranosyl.

In one embodiment, R1 is selected from: C₁-C₅ acyl or —OC(═O)(CHR8)ᵣ—R7, where r is 1, 2, 3, 4, 5, 6, 7, or 8. In one embodiment, —(CHR8)ᵣ — is a straight- or branched-chain alkylene, where r is 1, 2, 3, 4, 5, 6, 7, or 8. In another embodiment, R7 is an morpholinyl, e.g., —N-morpholinly. In one embodiment R1 is —C(═O)—C₁-C₈-alkylene-N-morpholinly, —C(═O)(CH₂)ᵣ—R7, or —C(═O)—(CH₂)ᵣ—N-morpholinyl; where r is 1, 2, 3, 4, 5, 6, 7, or 8.

In one embodiment, R1 is selected from: acetyl, propionyl, 3-hydroxy-2-methylpropionyl, tetrahydropyranyl, —C(O)—(CH₂)ᵣ—C(O)OH, —C(O)—(CH₂)ᵣ—OR18, —C(O)—(CHR18)ᵣ—C(O)OH, —C(O)—(CHR18)ᵣ—OR19, —C(O)—(CR18R19)ᵣ—OR20, —C(O)O—(CH₂)ᵣ—OR18, —C(O)CH₂—(OCH₂CH₂)ᵣ—OR18, —C(O)C(O)(OCH₂CH₂)ᵣ—OR18, —C(O)—(CH₂)ᵣ—N(R18)R19, —C(O)OCH₂N(R18)R19, —C(O)NH—(CH₂)ᵣ—N(R18)R19, —C(O)—(CH₂)ᵣ—N⁺(R18)(R19)(R20)X⁻, —C(O)O(CH₂)ᵣ—N⁺(R18)(R19)(R20)X⁻, —C(O)NH—(CH₂)ᵣ—N⁺(R18)(R19)(R20)X⁻, L-amino acid residue, D-amino acid residue, beta-amino acid residue, gamma-amino acid residue, —P(O)(OR21)(OR22), or —P(O)(NR18)(NR19); wherein R18, R19, and R20 are each independently selected from the group consisting of: —OH, formyl, acetyl, pivaloyl, —NH₂, —NH(CH₃), —NH(CH₂CH₃), N(CH₃)₂, —NH[C(O)H], —NH[C(O)CH₃], and C₃-C₅-alkyl; wherein r is 1, 2, 3, 4, 5, 6, 7, or 8; wherein X⁻ is a counter ion derived from a pharmaceutically acceptable acid; and wherein R21 and R22 are each independently selected from the group consisting of —H, C₁-C₅-alkyl, alkali metal cations, alkaline earth metal cations, ammonium cation, methyl ammonium cation, and pharmaceutically acceptable bases.

In one embodiment, R1 is: —C(O)C(R23)(CH₂)₂R24; wherein R23 is —H or a straight-chained, branched or cyclic C₁-C₆ lower alkyl; and, R24 is a 5-7 membered heterocyclic ring (e.g., morpholinyl) in which at least one of the member atoms is N or NR25 where R25 is H₂, H₃⁺, or mono- or dialkyl C₁-C₆, or a salt thereof.

In one embodiment, R1 is selected from: —C(O)(CH₂)₂N(CH₃)₂, —C(O)OCH₂N(CH₂CH₃)₂, —C(O)NHCH₂CH₃, —C(O)CH₂N(CH₃)₂, —C(O)(CH₂)₂OCH₂CH₃, —C(O)NH(CH₂)₄NH₂, —C(H)O, —C(O)CH₂OH, —C(O), —C(O)(CH₂OCH₂)₃CH₂OCH₃, —C(O)OCH₃, —C(O)CH₃, —C(O)(CH₂OCH₂)₂CH₂OCH₃, —C(O)CH(OH)CH₂OH, —C(O)C(CH₃)(CH₂OH)CH₂OH, —C(O)(CH₂)₃C₆H₅NH₂, —C(O)CH(NH₂)[CH(CH₃)]₂CH₃, —P(O)(ONH₄)₂, —C(O)(CH₂)₃C₆H₅NHC(O)(CH₂)₂C(O)OH, —C(O)CH[NHC(O)(CH₂)₂C(O)OH]CH(CH₃)CH₃, —C(O)[CH(OH)]₄, —C(O)CH₂N(CHO)CH₃, —C(O)([O(CH₂)₂]₄CH₃), —C(O)C₄H₄NH, —C(O)C(O)([O(CH₂)₂]₄OCH₃), 2-amino-3-(4-hydroxyphenyl)-propanalyl, —C(O)(CH₂)₂NH₂, —C(O)CH₂NHCO, —C(O)CH₂NH₂, 2-amino-3-(1H-indol-3-yl)-propanalyl, —C(O)CH₂NH CH₃, —C(O)(CH₂)₃N[CH(CH₃)CH₃)]₂, —C(O)CH₂NHC(O), —C(O)CH(NH₂)CH(CH₃)CH₃, —C(O)OCH₂CH(OH)CH(OH), —C(O)[CH(OH)]₃CH₂OH, —C(O)CH(OH)CH₂OH, —C(O)OCH₂CH(OH)CH₂OH, or —C(O)CH(NH₂)CH₂CH₂CH(OH)NH₂.

In one embodiment, R1 is selected from: —C(O)CH₂NHC(O)—R26, —C(O)CH₂NHC(O)—R26, —C(O)CH₂NHC(O)—R26, —C(O)CH(CH₃)NHC(O)—R26, —C(O)CH(CH₃)NHC(O)—R26, —C(O)CH(CH₃)NHC(O)—R26, —C(O)CH(CH₃)NHC(O)—R26, —C(O)CH₂CH₂NHC(O)—R26, —C(O)CH₂CH₂NHC(O)—R26, —C(O)CH₂CH₂NHC(O)—R26, —C(O)CH₂CH₂NHC(O)—R26, —C(O)CH(NH₂)CH₂CH₂C(O)NHCH₃, —C(O)CH(NH₂)CH₂CH₂C(O)NHCH₂CH₃, —C(O)CH(NH₂)CH₂C(O)NHCH₃, or —C(O)CH(NH₂)CH₂C(O)NHCH₂CH₃; wherein R26 is either phenyl or pyridinyl. In a further embodiment, R26 is selected from 2-pyridinyl, 3-pyridinyl, or 4-pyridinyl.

In one embodiment, R1 is selected from: —C(O)CH₂NHC(O)-2-pyridinyl, —C(O)CH₂NHC(O)-3-pyridinyl, —C(O)CH₂NHC(O)-4-pyridinyl, —C(O)CH(CH₃)NHC(O)-phenyl, —C(O)CH(CH₃)NHC(O)-2-pyridinyl, —C(O)CH(CH₃)NHC(O)-3-pyridinyl, —C(O)CH(CH₃)NHC(O)-4-pyridinyl, —C(O)CH₂CH₂NHC(O)-phenyl, —C(O)CH₂CH₂NHC(O)-2-pyridinyl, —C(O)CH₂CH₂NHC(O)-3-pyridinyl, —C(O)CH₂CH₂NHC(O)-4-pyridinyl, —C(O)CH(NH₂)CH₂CH₂C(O)NHCH₃, —C(O)CH(NH₂)CH₂CH₂C(O)NHCH₂CH₃, —C(O)CH(NH₂)CH₂C(O)NHCH₃, or —C(O)CH(NH₂)CH₂C(O)NHCH₂CH₃.

In one embodiment, R1 is selected from any one of:

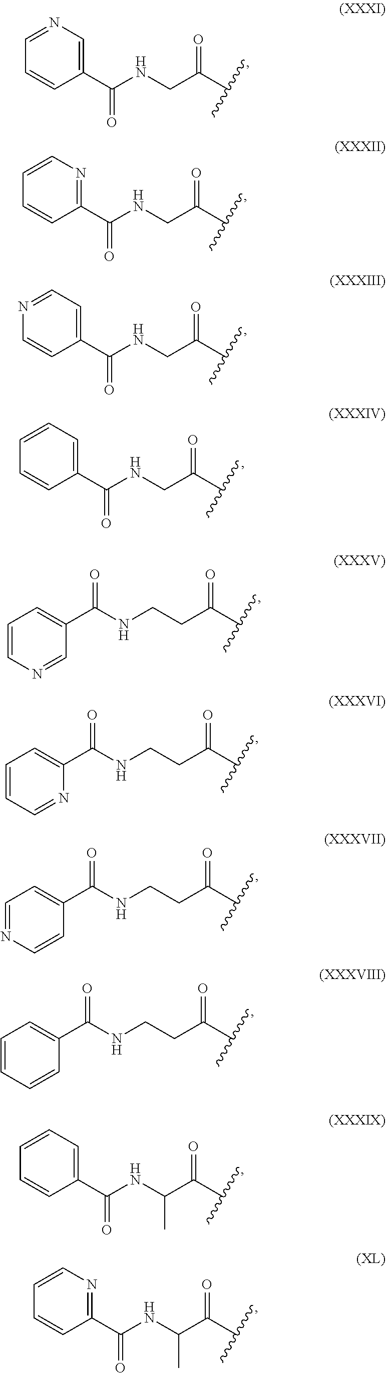

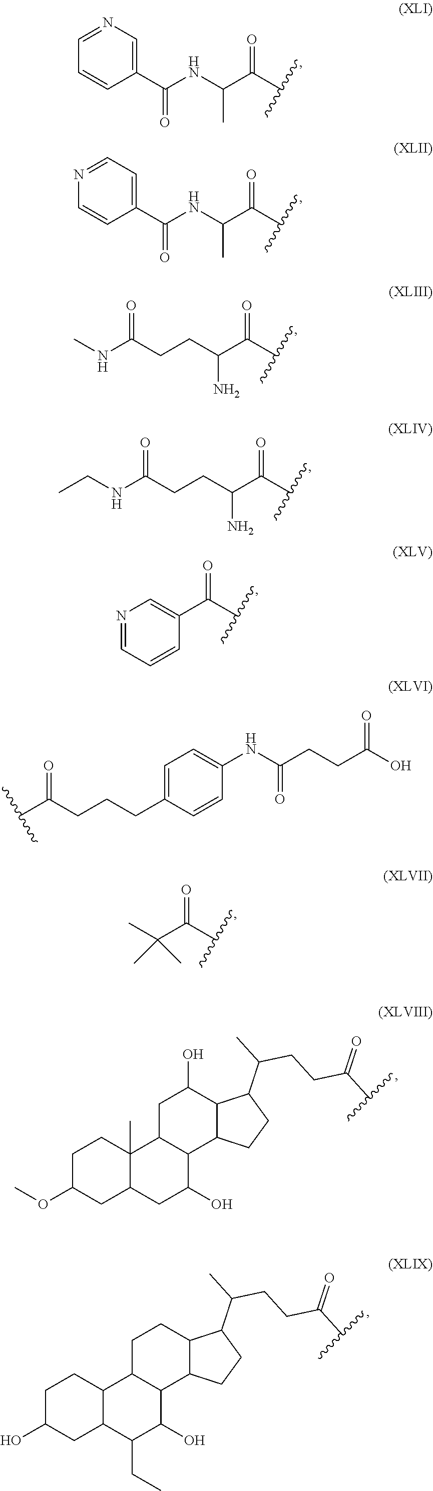

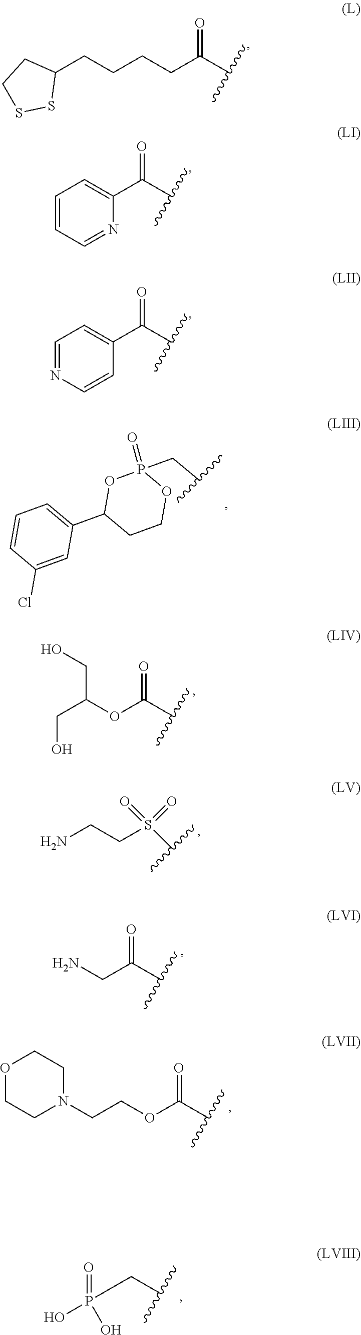

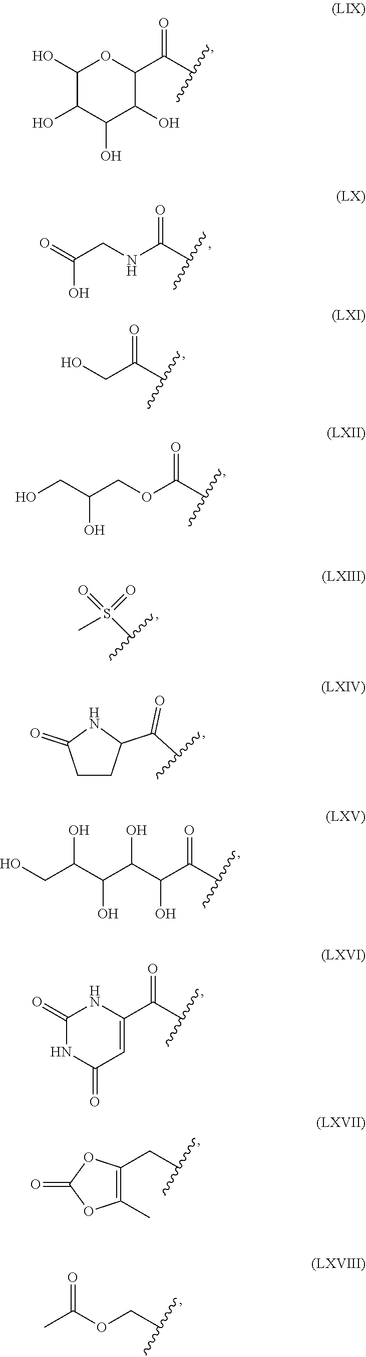

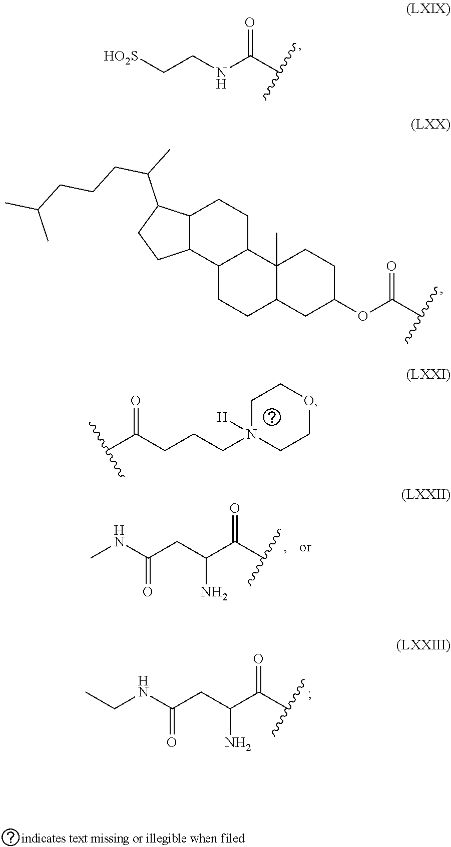

wherein the wavy line represents a radical.

In a preferred embodiment, R1 is a radical selected from XXXI-XLII. In a more preferred embodiment, R1 is a radical selected from XXXI-XXXVIII. In a more preferred embodiment, R1 is XXXI.

In one embodiment, R1 is selected from any one of:

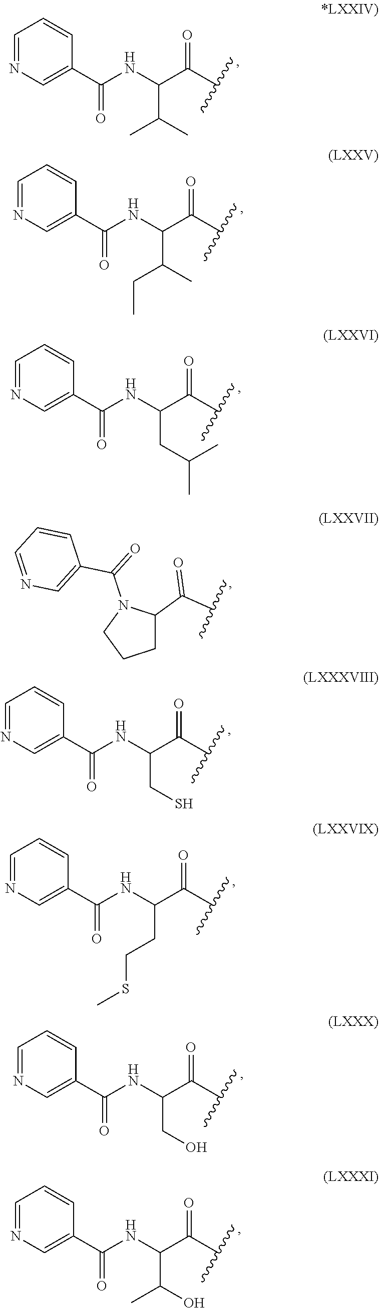

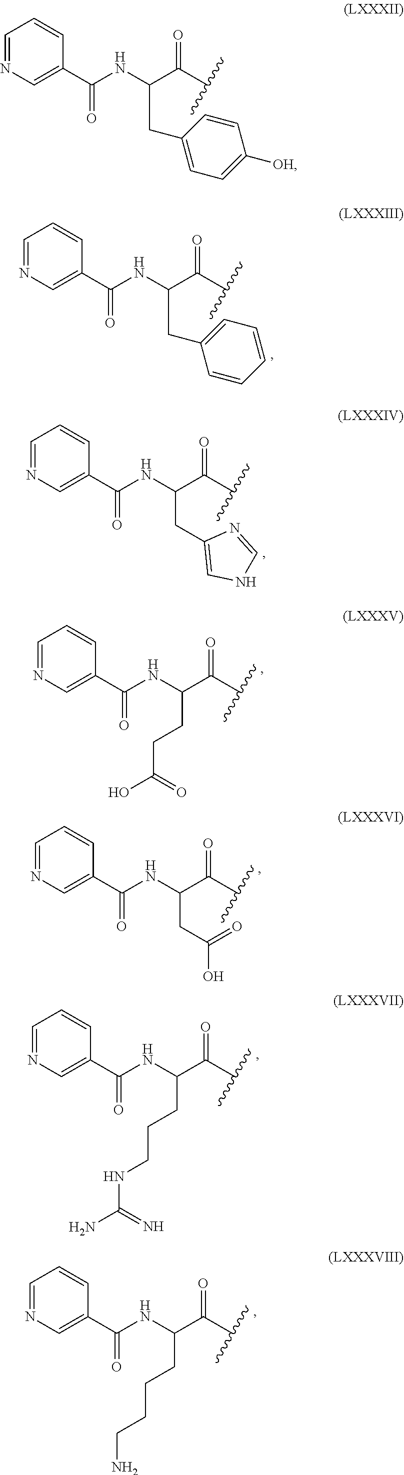

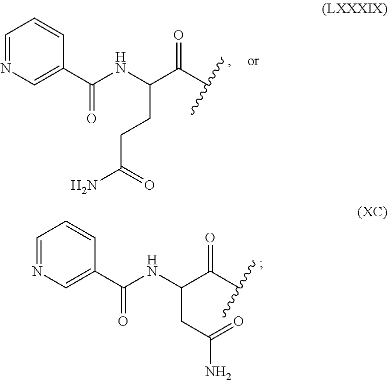

wherein the wavy line represents a radical.

In one embodiment, R1 is selected from any one of:

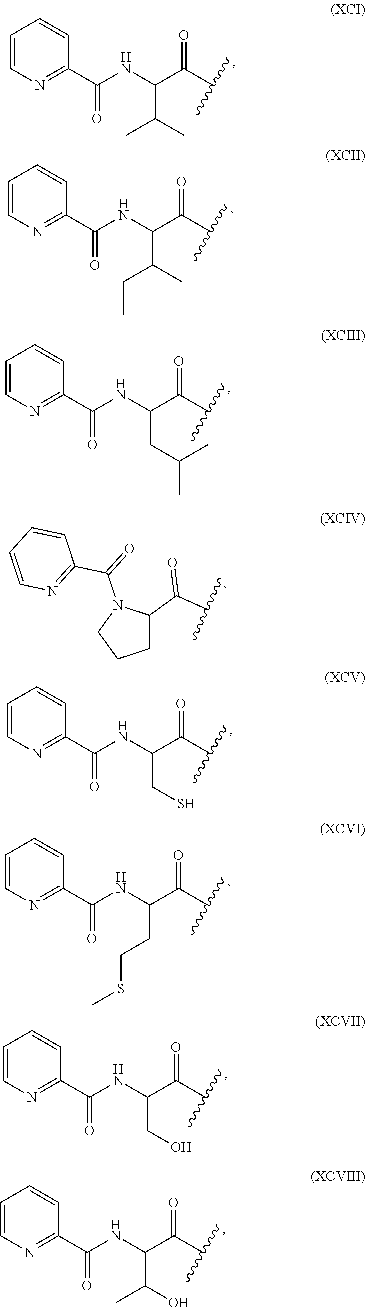

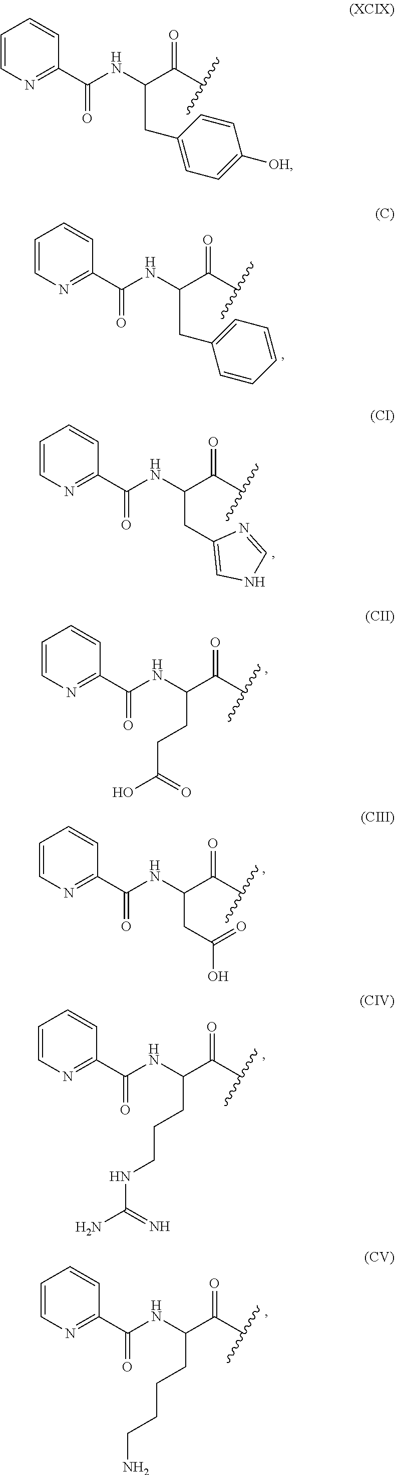

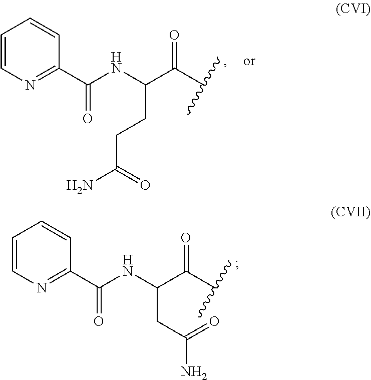 [SMILES: *C(=O)C(CCC(N)=O)NC(=O)c1ccccn1 ; *C(=O)C(CC(N)=O)NC(=O)c1ccccn1]

wherein the wavy line represents a radical.

In one embodiment, R1 is selected from any one of:

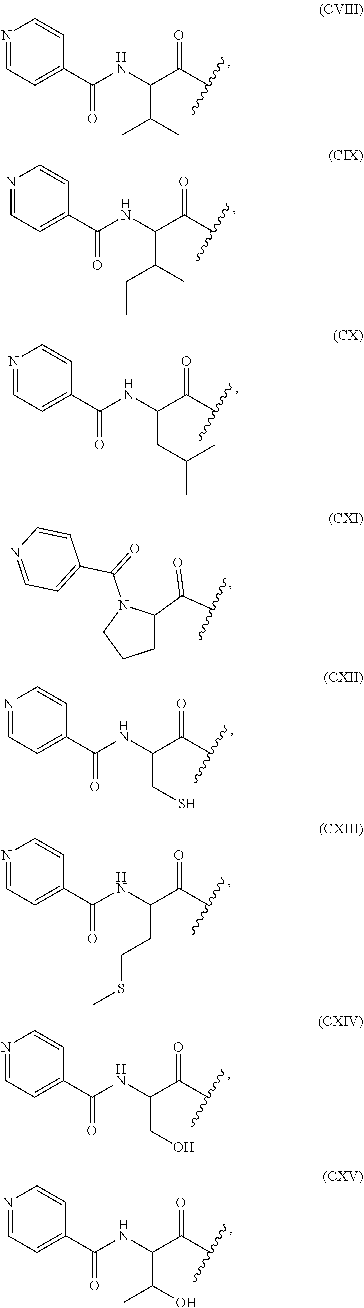

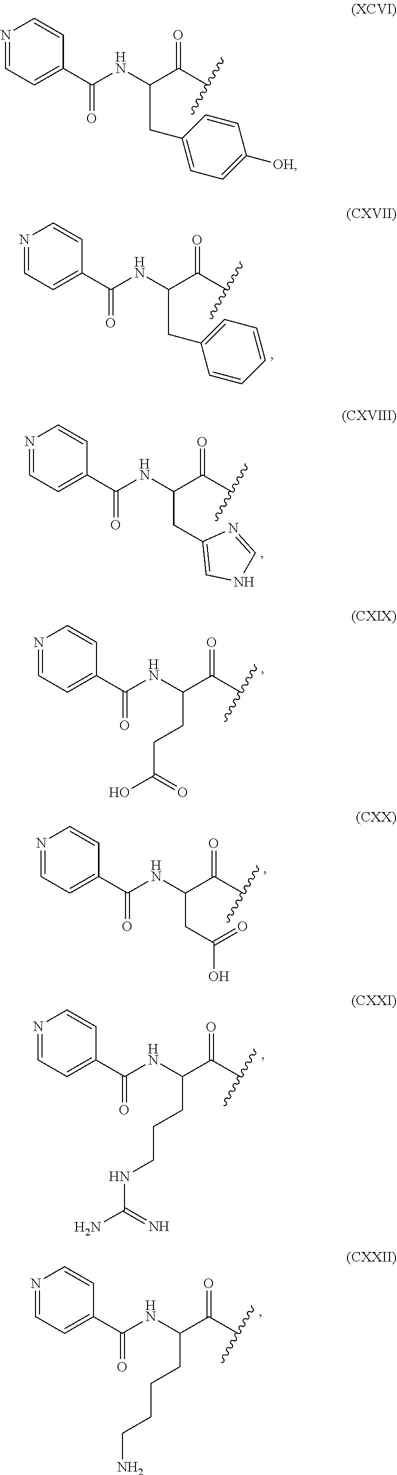

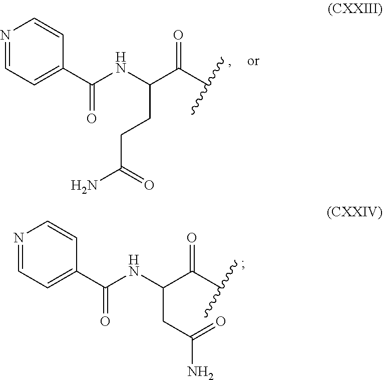 [SMILES: *C(=O)C(CCC(N)=O)NC(=O)c1ccncc1 ; *C(=O)C(CC(N)=O)NC(=O)c1ccncc1]

wherein the wavy line represents a radical.

In one embodiment, R1 is selected from any one of:

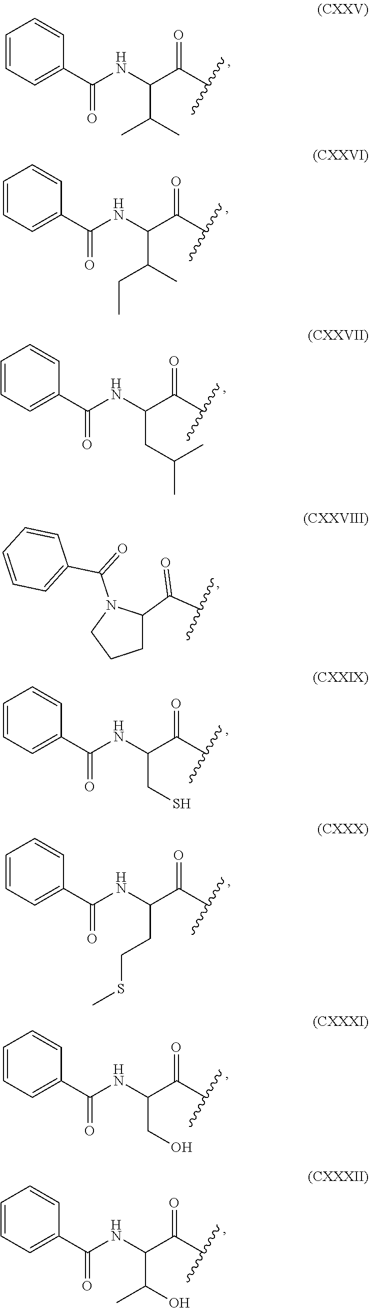

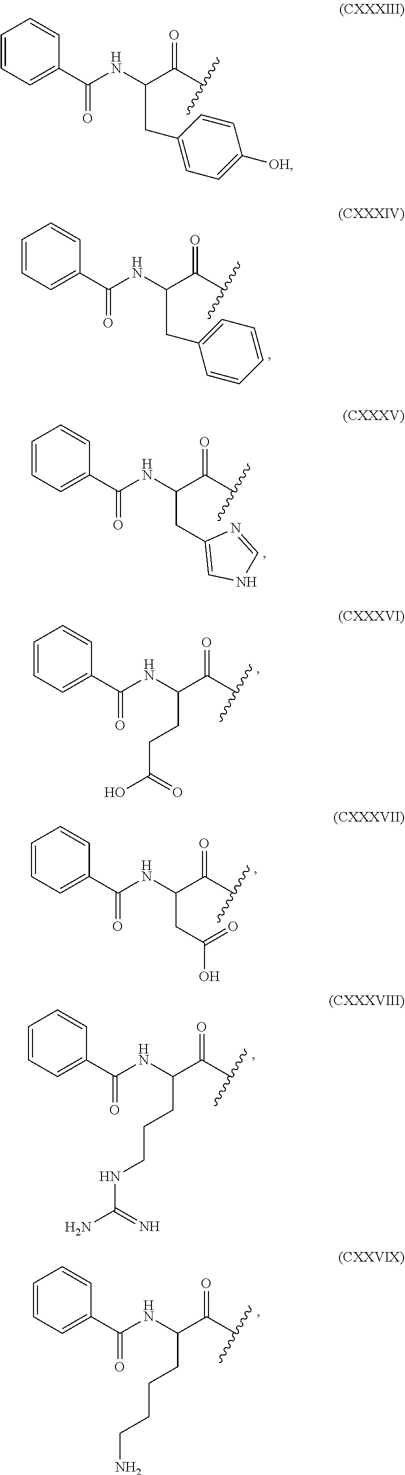

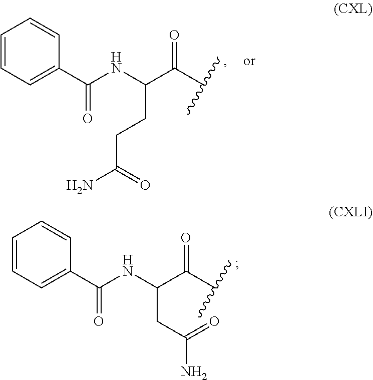 [SMILES: *C(=O)C(CCC(N)=O)NC(=O)c1ccccc1 ; *C(=O)C(CC(N)=O)NC(=O)c1ccccc1]

where the wavy line represents a radical.

In one embodiment, R1 is selected from any one of:

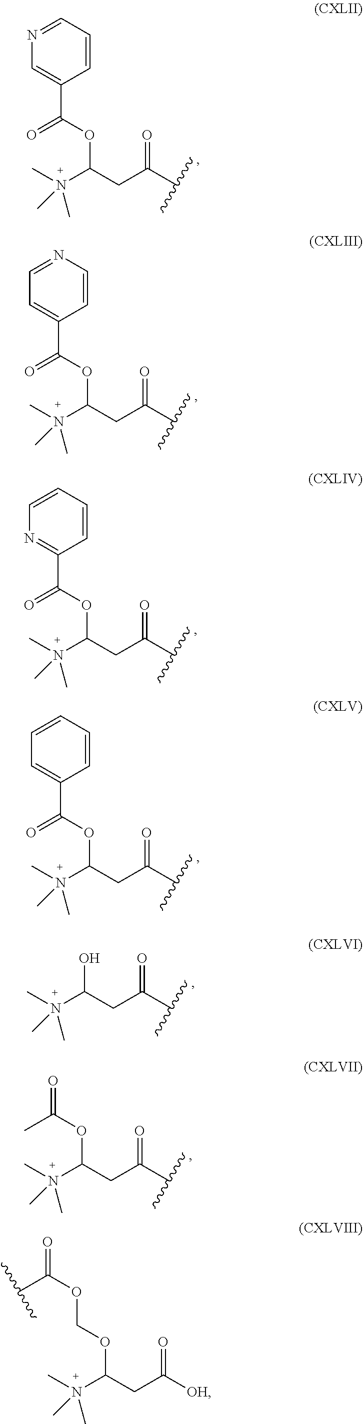

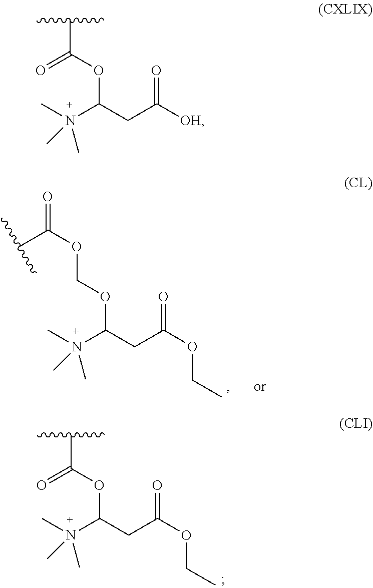

where the wavy line represents a radical.

In one embodiment, R1 is selected from any one of:

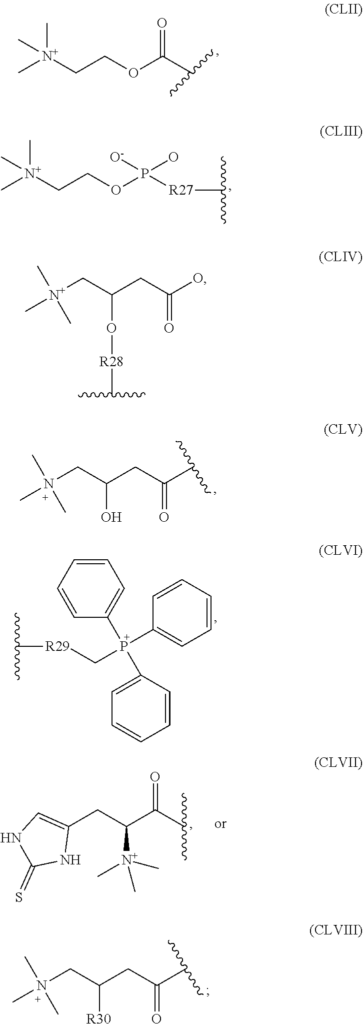

wherein the wavy line represents a radical, and wherein:
R27 is selected from: a bond (absent) or —O—(C₁-C₁₅ alkylene)-C(O)—, wherein said alkylene is saturated or unsaturated, substituted or unsubstituted;
R28 is —COC(O)—, —C(O)—, —CH₂C(O)—, —CH₂C(O)—(C₁-C₁₅ alkylene)-C(O)— or —C(O)—(C₁-C₁₅ alkylene)-C(O)—, wherein said alkylene is saturated or unsaturated, substituted or unsubstituted;
R29 is selected from: a bond (absent) or —(C₁-C₁₅ alkylene)-C(O)—, wherein said alkylene is saturated or unsaturated, substituted or unsubstituted; and
R30 is selected from: hydroxyl, —OC(O)-2-pyridinyl-, —OC(O)-3-pyridinyl-, —OC(O)-4-pyridinyl-, or —OC(O)H.

In one embodiment, said C₁-C₁₅ alkylene of R27, R28 or R29 is independently selected from C₁, C₂, C₃, C₄, C₅, C₆, C₇, C₈, C₉ C₁₀, C₁₁, C₁₂, C₁₃, C₁₄, or C₁₅ alkylene. In one embodiment R27 is —O—(CH₂)_{q}—C(O)—; wherein q is 1, 2, 3, 4, 5, 6, 7, 8, 9, 10, 11 12, 13, 14 or 15. In another embodiment R28 is —C(O)—(CH₂)_{q}—C(O)—; wherein q is 1, 2, 3, 4, 5, 6, 7, 8, 9, 10, 11 12, 13, 14 or 15.

In one embodiment, R1 is selected from:

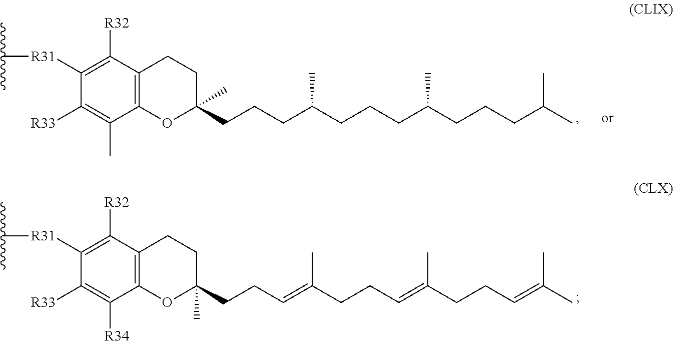

wherein the wavy line represents a radical, and wherein:
R31 is —C(O)(CH₂)ₚC(O)O—;
R32, R33 and R34 are each independently selected from —H or —CH₃; and wherein p is 1, 2, 3, 4, 5, 6, 7, 8, 9 or 10.

In one embodiment, R1 is selected from:

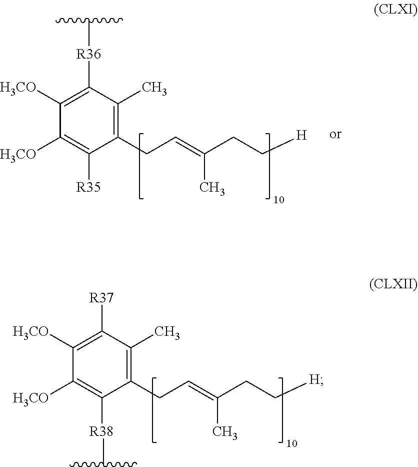

wherein the wavy line represents a radical, and wherein:
R36 and R38 are —OC(O)(CH₂)ₚC(O)—, wherein R36 and R38 are bound to their respective aryl rings of (CLXI) and (CLXII) through the carboxyl radical and the xanthophyll is bound to the acyl radical;
R35 is selected from: ═O (oxo), —OH or the same —R36-xanthophyll substituent on the opposite side of the aryl ring of (CLXI); and
R37 is selected from: ═O (oxo), —OH or the same —R38-xanthophyll substituent on the opposite side of the aryl ring of (CLXII); and wherein p is 1, 2, 3, 4, 5, 6, 7, 8, 9 or 10.

In one embodiment, the xanthophyll derivative of the present invention is a compound or a pharmaceutically acceptable salt, co-crystal, geometric isomers or stereoisomers thereof, selected from the group consisting of:

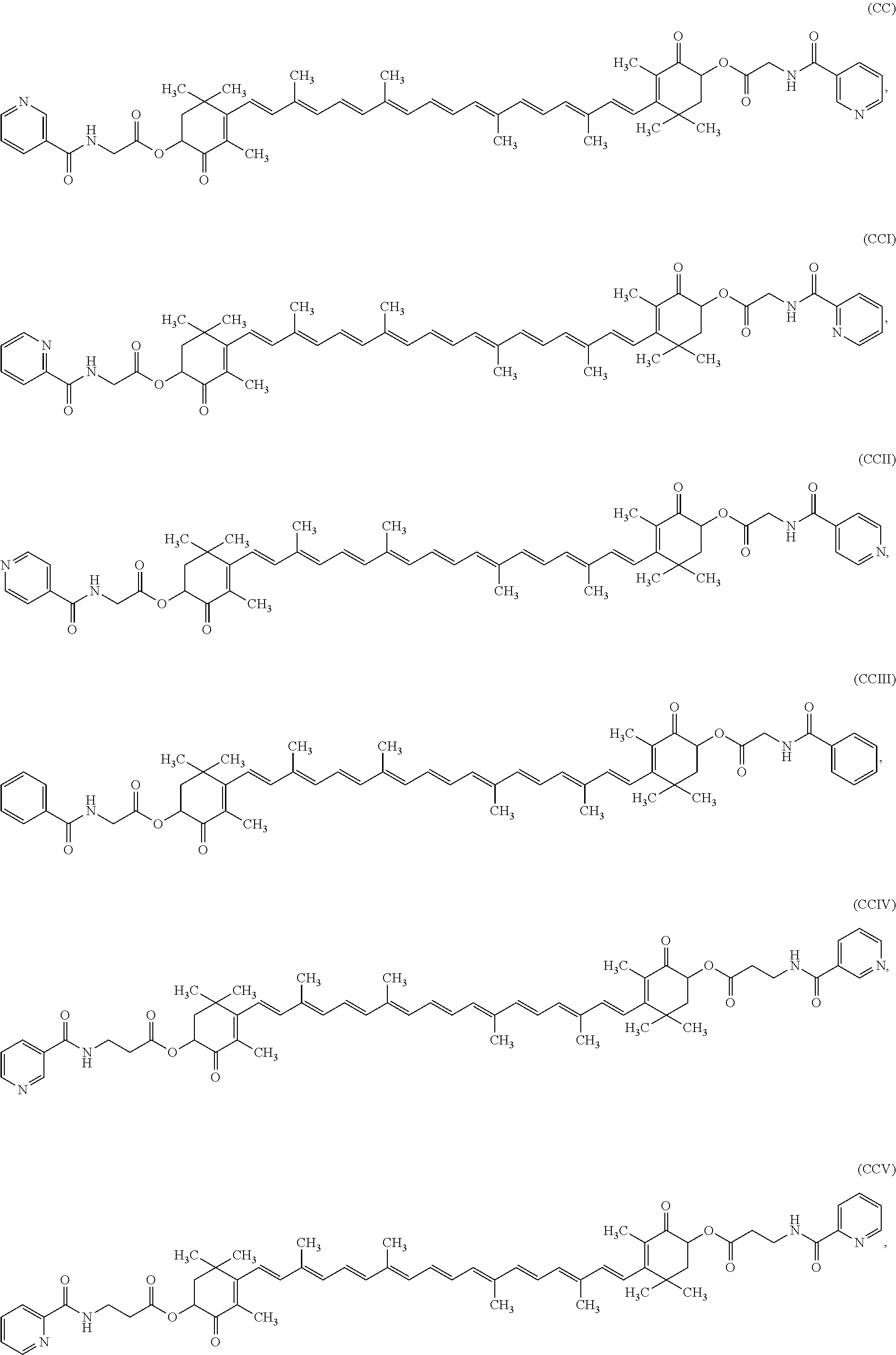

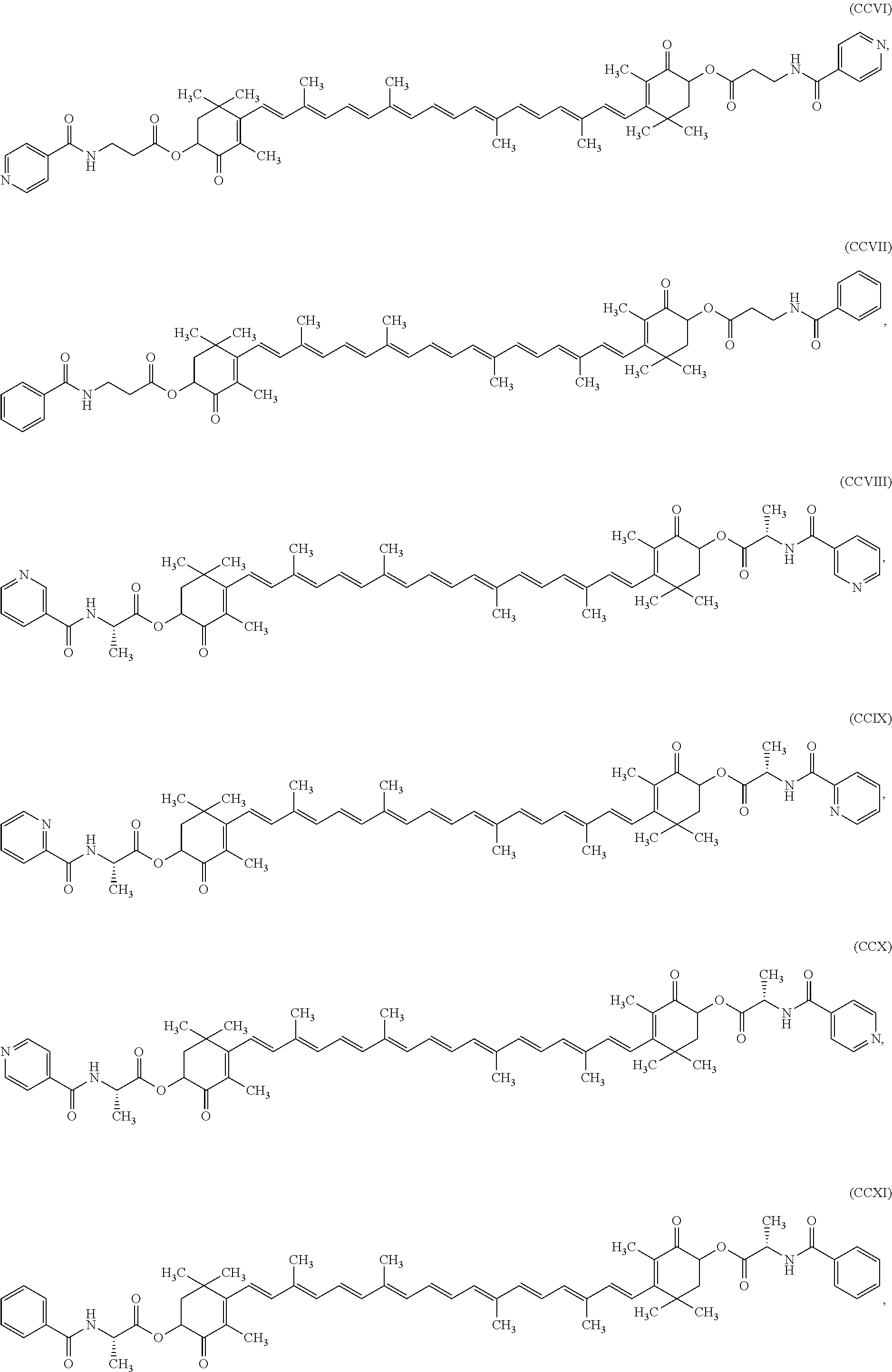

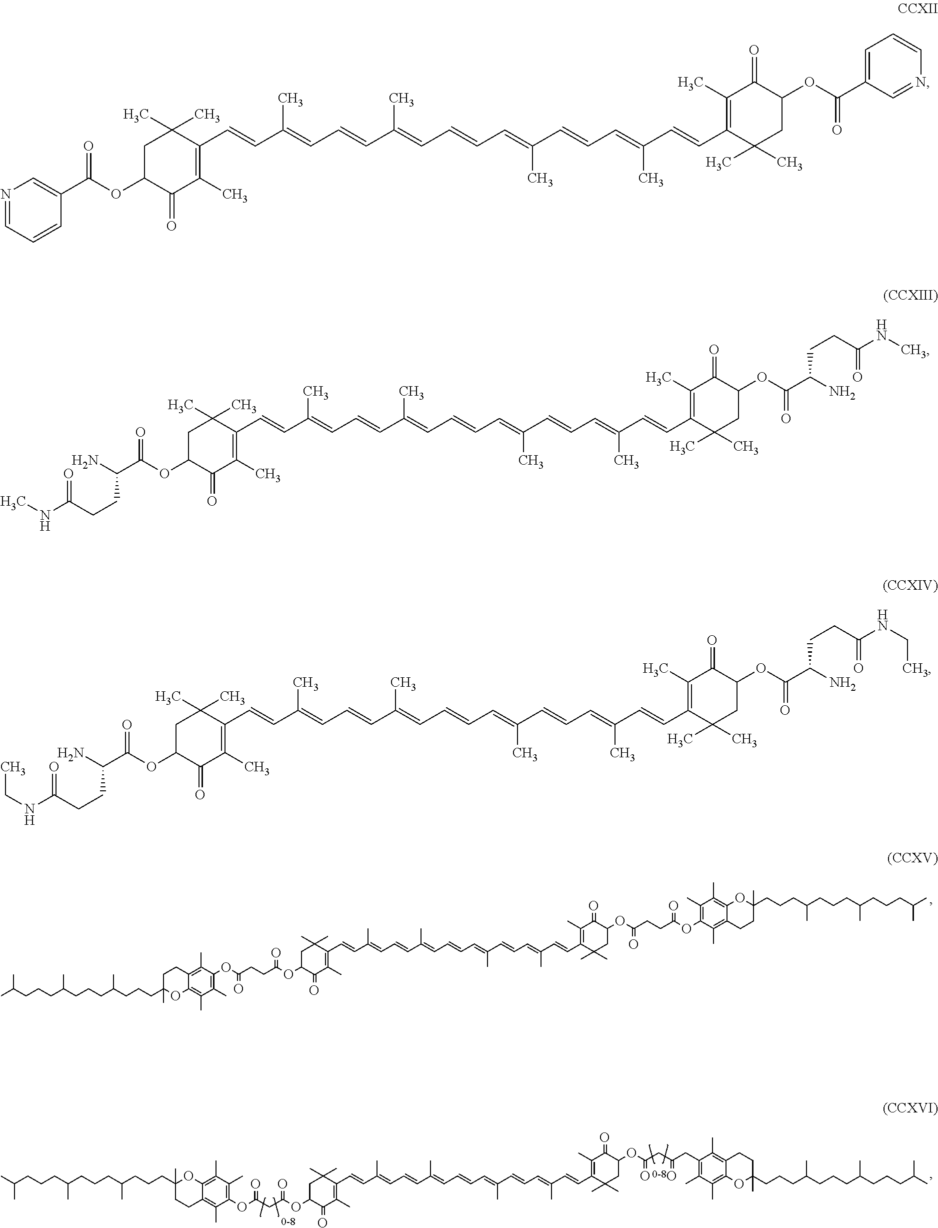

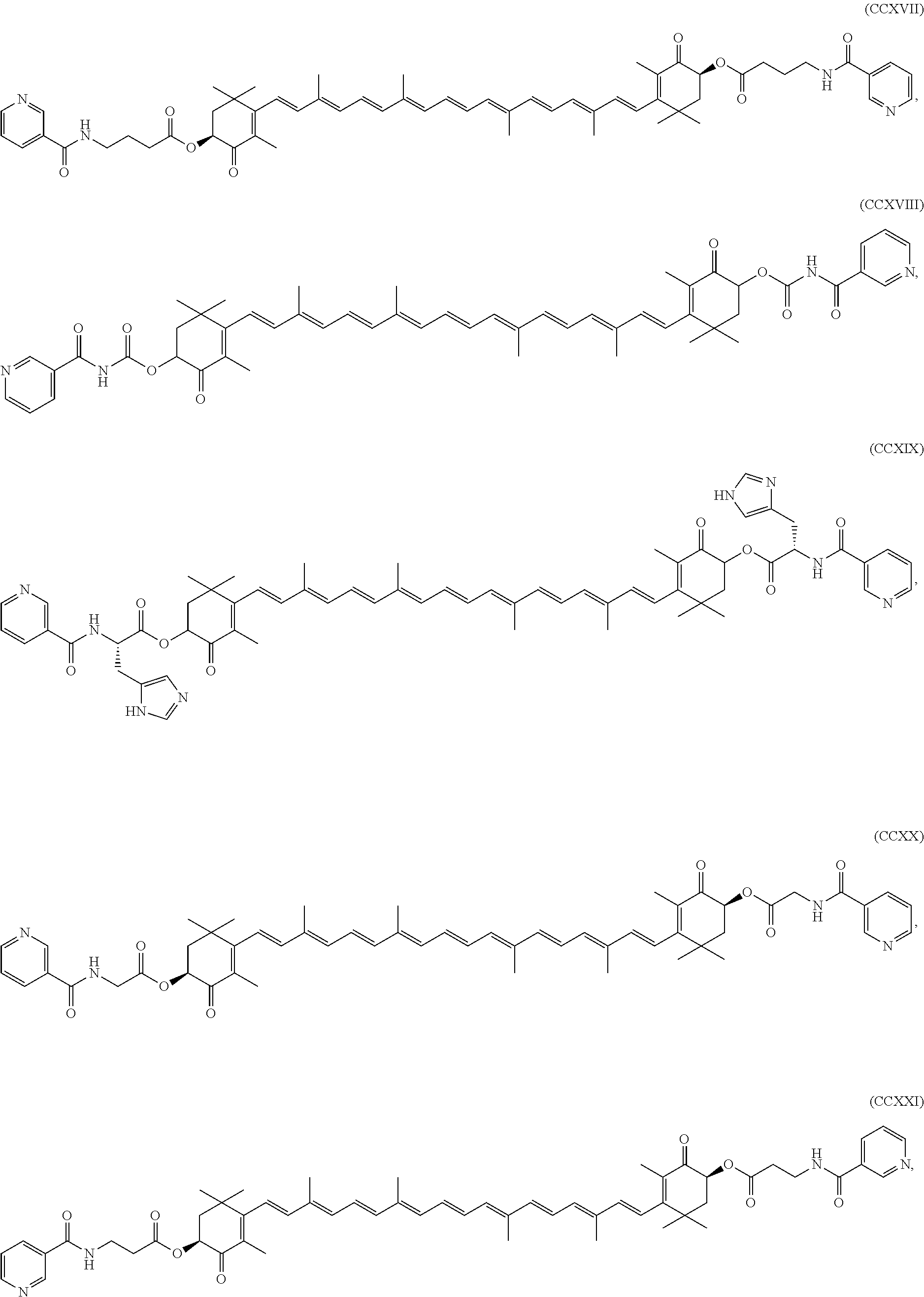

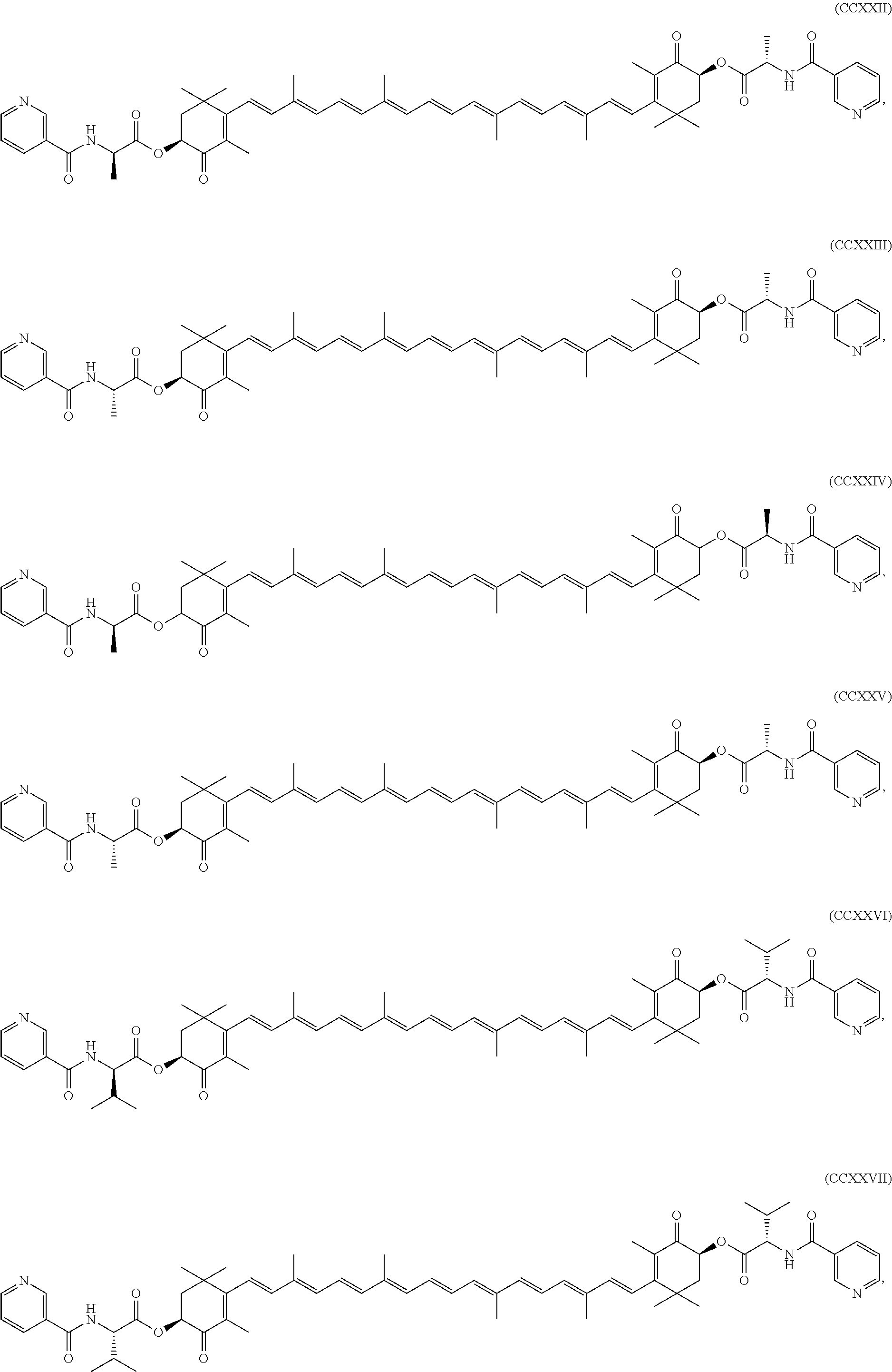

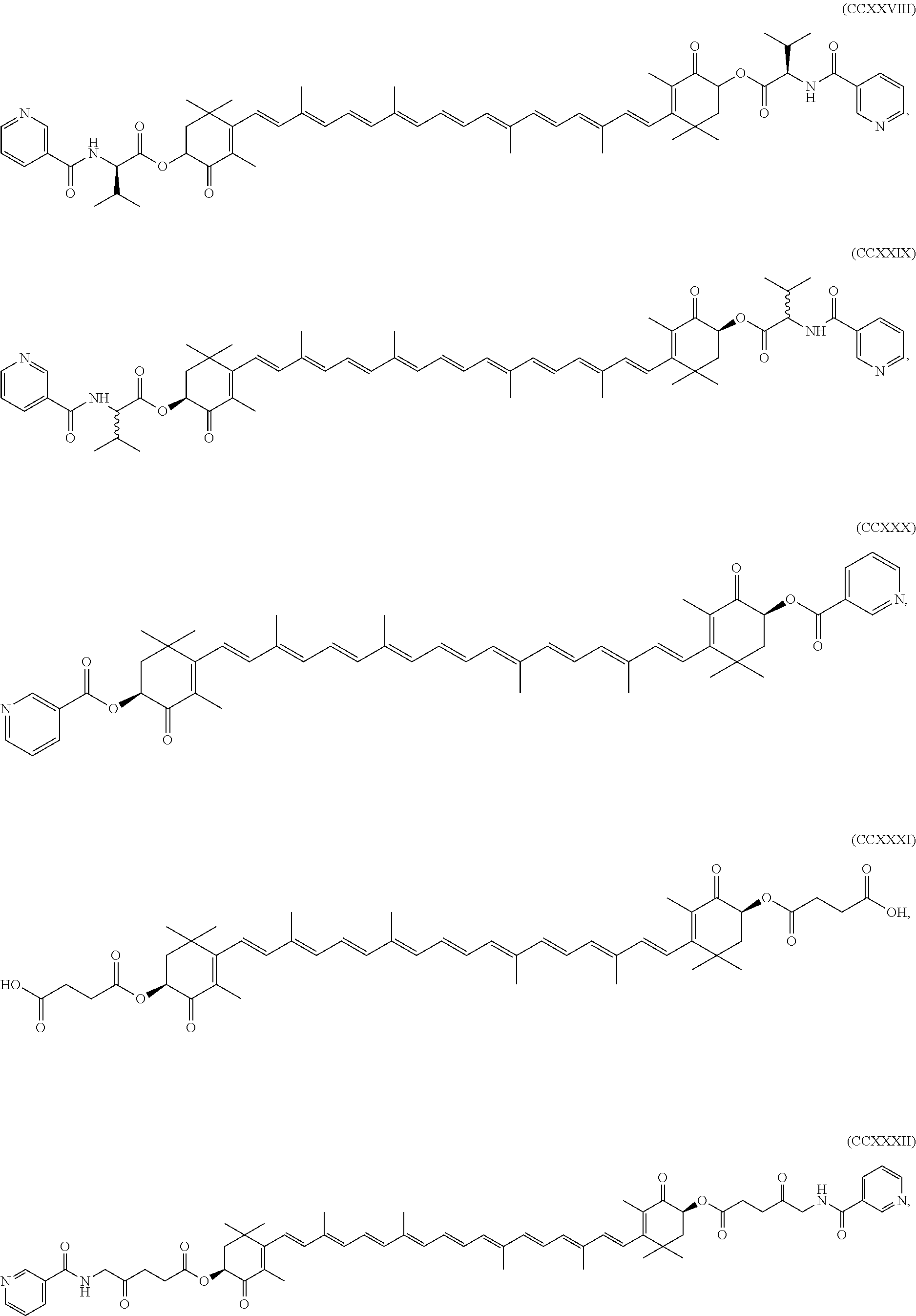

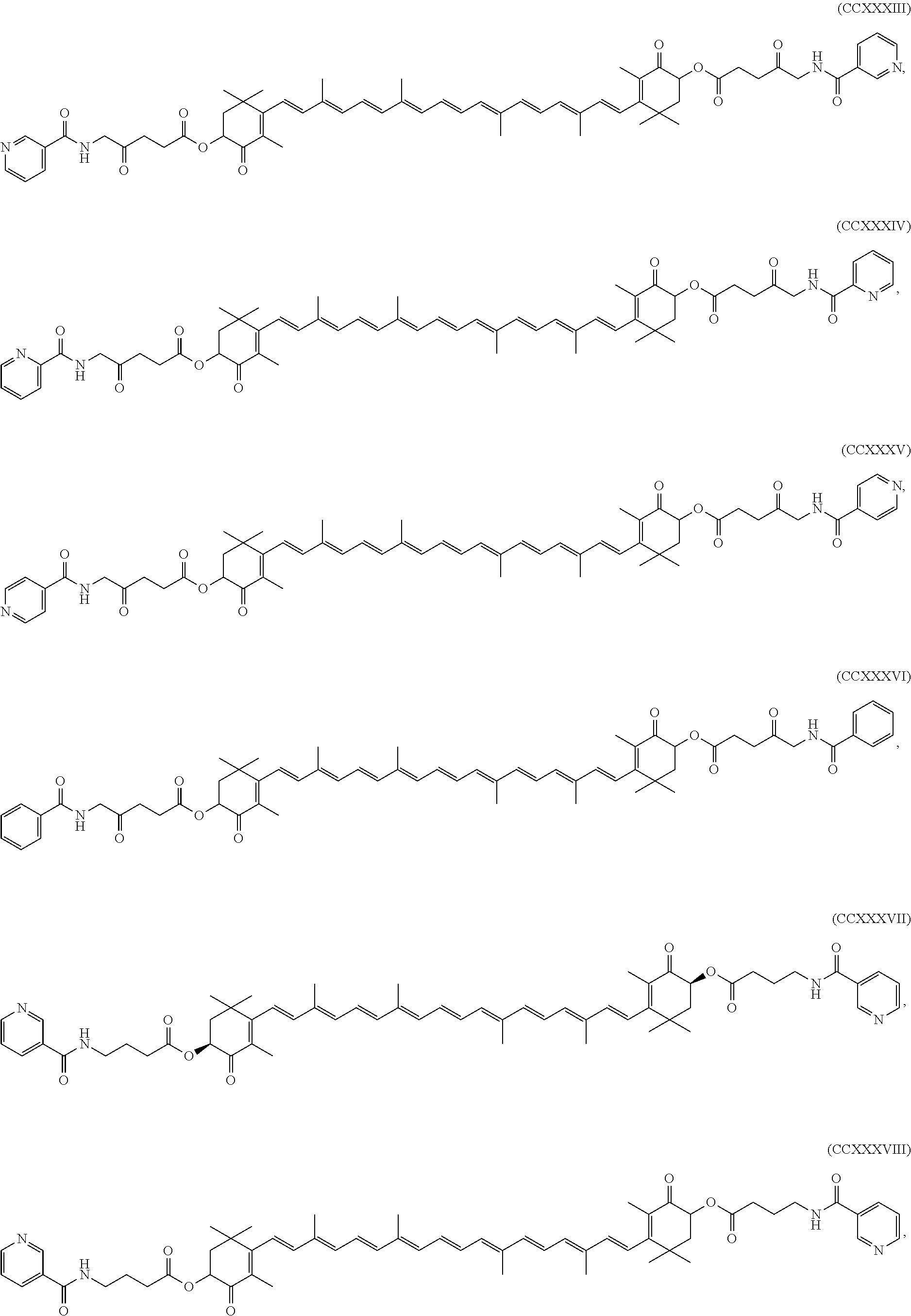

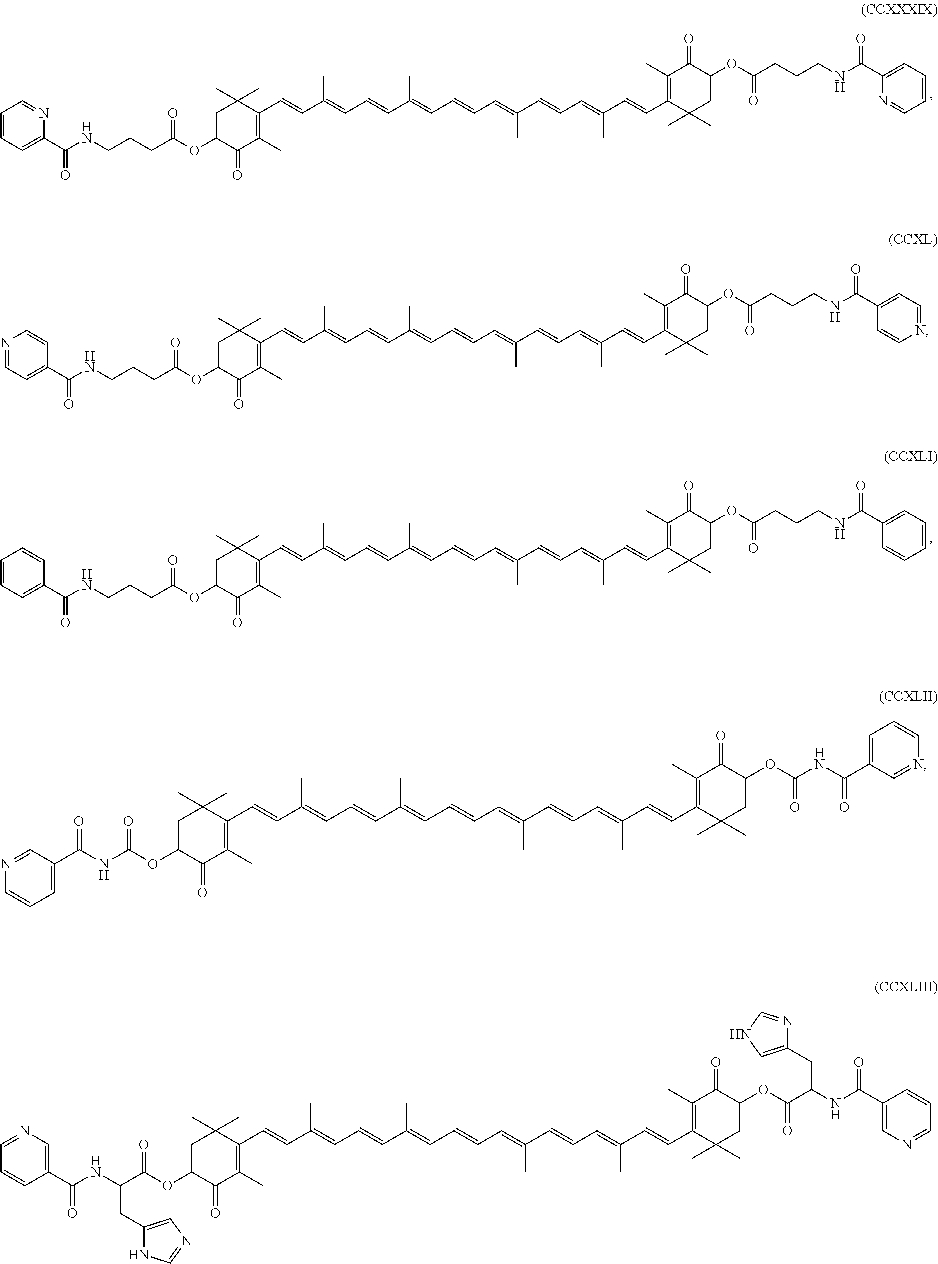

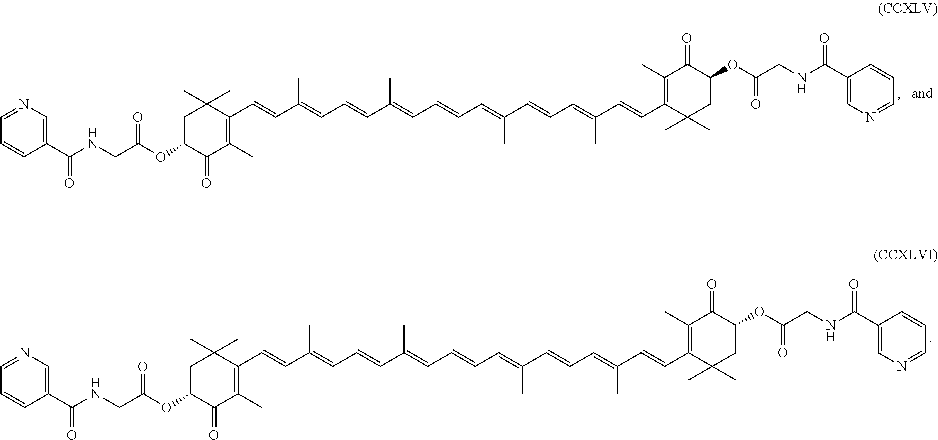 [SMILES: CC1=C(/C=C/C(C)=C/C=C/C(C)=C/C=C/C=C(C)/C=C/C=C(C)/C=C/C2=C(C)C(=O)[C@H](OC(=O)CNC(=O)c3cccnc3)CC2(C)C)C(C)(C)C[C@H](OC(=O)CNC(=O)c2cccnc2)C1=O ; CC1=C(/C=C/C(C)=C/C=C/C(C)=C/C=C/C=C(C)/C=C/C=C(C)/C=C/C2=C(C)C(=O)[C@H](OC(=O)CNC(=O)c3cccnc3)CC2(C)C)C(C)(C)C[C@@H](OC(=O)CNC(=O)c2cccnc2)C1=O]

In a preferred embodiment, the xanthophyll derivative is a compound having a chemical structure selected from any one of CC-CCXI, CCXIII, CCXIV, CCXVII-CCXXIX, and CCXXXII-CCXLIII. In a more preferred embodiment, the xanthophyll derivative is a compound having a chemical structure selected from any one of CC-CCXI, CCXIII, CCXIV, CCXVII-CCXXV, and CCXXXII-CCXLIII. In a more preferred embodiment, the xanthophyll derivative is a compound having a chemical structure selected from any one of CC, CCIV, CCVIII, CCXVII, CCXX-CCXXII, CCXXXII, CCXXXVII, CCXXXVIII or CCXLIII. In a more preferred embodiment, the xanthophyll derivative is a compound having a chemical structure selected from any one of CC, CCIV, CCVIII, CCXVII, CCXX, CCXXI, CCXXII, CCXXXVII, or CCXXVIII. In a more preferred embodiment, the xanthophyll derivative is a compound having a chemical structure selected from CC. In a more specific embodiment, the xanthophyll derivative is a compound having a chemical structure selected from CCXX, CCXLV or CCXLVI. In a more preferred embodiment, the xanthophyll derivative is a compound having a chemical structure of CCXX.

In one embodiment, R1 is selected from any one of:

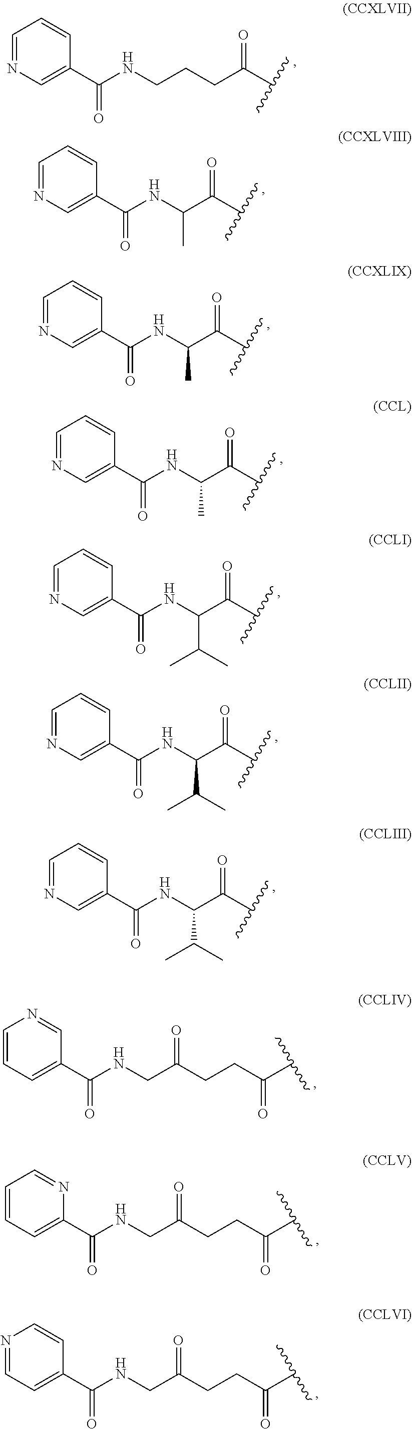

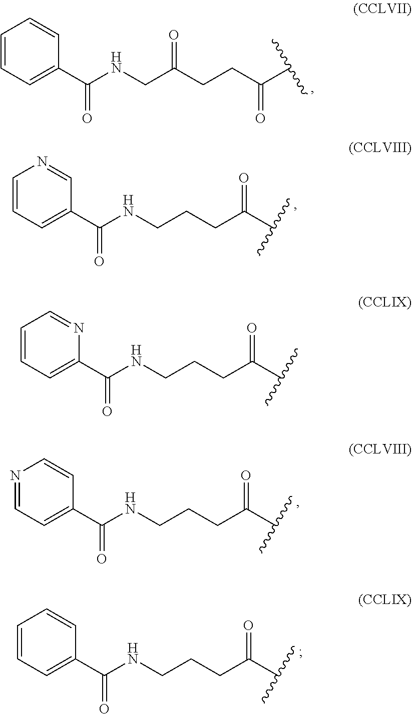 [SMILES: *C(=O)CCC(=O)CNC(=O)c1ccccc1 ; *C(=O)CCCNC(=O)c1cccnc1 ; *C(=O)CCCNC(=O)c1ccccn1 ; *C(=O)CCCNC(=O)c1ccncc1 ; *C(=O)CCCNC(=O)c1ccccc1]

wherein the wavy line represents a radical.

In a preferred embodiment, R1 is selected from any one of: CCXLVII-CCL, and CCLIV-CCLIX. In a more referred embodiment, R1 is selected from any one of CCLIV-CCLIX. In a more preferred embodiment, R1 is CCXLVII.

In one embodiment, the composition is a pharmaceutical composition. In another embodiment, the composition is a foodstuff. In another embodiment, the composition is a food additive. In another embodiment, the composition is a food supplement. In another embodiment, the composition is a feedstuff. In another embodiment, the composition is a feed additive.

The xanthophyll derivatives of the present invention comprise a modification to at least one hydroxyl group present on a parent xanthophyll. In one embodiment, the hydroxyl group is substituted with a prodrug moiety.

In one embodiment, the xanthophyll derivative is a salt or co-crystal. In one embodiment, the salt is selected from: an aluminum, arginine, benzathine, calcium, chloroprocaine, choline, diethanolamine, ethanolamine, ethylenediamine, lysine, magnesium, histidine, lithium, meglumine (N-methyl-glucamine), potassium, procaine, sodium, triethylamine, tributylamine, tromethamine (TRIS), strontium or zinc salt.

In another embodiment, the salt is selected from: an acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydrochloride, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate or tosylate salt.

In another embodiment, the salt is an amino acid salt, e.g., lysine, ornithine or arginine. In one embodiment, the xanthophyll derivative is a co-crystal of a pharmaceutically acceptable acid or base, an amino acid, or a pharmaceutically acceptable salt of sodium, potassium, magnesium, zinc, iron or aluminum. In another embodiment, the xanthophyll derivative is a co-crystal, wherein the co-crystal former is selected from the group consisting of: aconitic acid, adipic acid, alpha-tocopherol acetate, ascorbyl palmitate (palmitoyl L-ascorbic), benzoic acid, biotin, carotene (beta-carotene), cholic acid, choline bitartrate, choline chloride, citric acid, D- or DL-calcium pantothenate, D- or DL-sodium pantothenate, D-pantothenyl alcohol, desoxycholic acid, glycocholic acid, inositol, L-ascorbic acid, L-malic acid, L(+)-calcium lactate, L(+)-lactic acid, L(+)-potassium acid tartrate, L(+)-sodium potassium tartrate, L(+)-sodium tartrate, L(+)-tartaric acid, lecithin, linoleic acid, magnesium gluconate, magnesium stearate, malic acid, mannitol, niacin (nicotinic acid), niacinamide (nicotinamide), oleic acid, potassium gluconate, potassium glycerophosphate, potassium sorbate, propionic acid, propyl gallate, propyl paraben, propylene glycol, propylene glycol monostearate, pyridoxine, pyridoxine hydrochloride, riboflavin, riboflavin-5′-phosphate, sodium benzoate, sodium citrate, sodium erythorbate (sodium D-isoascorbate), sodium gluconate, sodium L-ascorbate, sodium oleate, sodium palmitate, sodium propionate, sodium sorbate, sorbic acid, sorbitol, stearic acid, stearyl citrate, succinic acid, sulfamic acid, taurocholic acid, thiamine hydrochloride, thiamine mononitrate, tocopherols, urea, vitamin b12 (cyanocobalamin), zinc gluconate, ferric citrate, ferrous ascorbate, ferrous citrate, ferrous fumarate, ferrous gluconate, ferrous lactate, L-glutamic acid, L-glutamic acid hydrochloride, monoammonium L-glutamate, monopotassium L-glutamate, monosodium L-glutamate, sucrose, vitamin A, vitamin A acetate, vitamin A palmitate, vitamin D2 (ergocalciferol), vitamin D3 (cholecalciferol), butylated hydroxyanisole (BHA), butylated hydroxytoluene (BHT), D(−)-lactic acid, lactic acid and caffeine.

In one embodiment, the composition comprises at least two active ingredients, wherein at least one of the active ingredients is a xanthophyll derivative, e.g., prodrug. In addition to the xanthophyll derivative, the composition may contain, e.g., one or more additional xanthophyll derivative, parent xanthophyll or other additional non-xanthophyll active ingredient(s). In some embodiments, the composition comprises at least two active ingredients, wherein at least one of the active ingredients is an astaxanthin derivative. In one embodiment, the composition comprises 2 or more astaxanthin derivatives.

In one embodiment, the xanthophyll, e.g., astaxanthin, derivative is administered in combination with an agent for treatment of non-alcoholic fatty liver disease or NASH. Agents used to treat non-alcoholic fatty liver disease include ursodeoxycholic acid (a.k.a., Actigall, URSO, and Ursodiol), metformin (Glucophage), Rosiglitazone (Avandia), Clofibrate, Gemfibrozil, Obeticholic acid and Elafibranor.

In one embodiment, the additional active ingredient(s) comprise a food supplement. In further embodiments, the food supplement is selected from any one or more of: 5-methyltetrahydrofolic acid, ademetionine, adenine, adenosine monophosphate, alfacalcidol, alpha-linolenic acid, ATP, beta carotene, biotin, calcidiol, calcitriol, castor oil, cholecalciferol, choline, chondroitin sulfate, coenzyme A, coenzyme Q10, resveratrol, creatine, curcumin, cyanocobalamin, cystine, dihomo-gamma-linolenic acid, ephedra, ergocalciferol, eucalyptol, fish oil, folic acid, ginkgo biloba, ginkgolide-A, ginkgolide-B, ginkgolide-C, ginkgolide-J, ginkgolide-M, ginseng, ginsenoside C, ginsenoside Rb1, ginsenoside Rg1, glutamic acid, glutathione, glycine, glycine betaine, histidine, hyperforin, icosapent, icosapent ethyl, eicosapentaenoate, eicosatetraenoate, docosahexaneoate, docosahexaneoate ethyl, inulin, kava, krill oil, L-Alanine, L-Arginine, L-Asparagine, L-Aspartic Acid, L-Citrulline, L-Cysteine, L-Glutamine, L-Isoleucine, L-Leucine, L-Lysine, L-Phenylalanine, L-Proline, L-Threonine, L-Tryptophan, L-Tyrosine, L-Valine, lipoic acid, lutein, melatonin, menadione, methionine, N-Acetylglucosamine, NAD+, NADH, niacin, octacosanol, omega-3 fatty acids, omega-6 fatty acids, ornithine, oxitriptan, oxogluric acid, pantothenic acid, phosphatidyl serine, phosphocreatine, prasterone, pyridoxal, pyridoxal phosphate, pyridoxine, pyruvic acid, riboflavin, sage oil, serine, serotonin, sesame oil, sinecatechins, spermine, St. John's Wort, succinic acid, taurine, tetrahydrofolic acid, thiamine, tretinoin, tyramine, ubidecarenone, ubiquinol, vitamin A, vitamin C, vitamin D, vitamin E, or vitamin K.

In one embodiment, the combined active ingredients in a composition of the present invention has synergistic activity, as compared to the additive activity of equivalent compositions comprising each active ingredient alone.

The compositions of the present invention further include xanthophyll derivatives that have at least one improvement selected from:
increased stability (e.g., in water, gastric or intestinal fluid, or at elevated temperature, e.g. at 100° C.), solubility, dissolution, oral bioavailability, Cmax, absorption, permeability into the enterocyte, transfer from the enterocyte to the blood (portal vein), transport into liver, uptake by hepatic cells including hepatocytes, non-parenchymal liver cells (Kupffer cells, sinusoidal endothelial cells, Ito-cells and/or liver-associated lymphocytes), perisinusoidal fat-storing cells, lipocytes, hepatic stellate cells, concentration of the xanthophyll derivative in hepatocytes Kupffer cells, sinusoidal endothelial cells, Ito-cells or liver-associated lymphocytes (preferential mass distribution), cholangiocytes of the biliary duct, interactions with and stabilization of mitochondria of cells, concentration of the xanthophyll derivative in perisinusoidal fat-storing cells, hepatic stellate cells or lipocytes; or
reduced time to Tmax, low intra-subject variability (including reduced food effect on absorption), lymphatic circulation or conversion to the parent xanthophyll in gastric and intestinal fluid (either chemically or enzymatically); or enhanced safety in human and animal disease treatment, including low liver toxic potential, preferential clearance mechanism sparing liver metabolism in advanced liver disease with loss of hepatic function.
in each case as compared to the parent xanthophyll, e.g., astaxanthin or xanthophyll isolated from a natural source, e.g., astaxanthin isolated from Haematococcus pluvialis. In one embodiment, the increase in solubility is selected from aqueous solubility, gastric fluid solubility or intestinal fluid solubility.

In one embodiment, the composition further comprises a pharmaceutically acceptable excipient.

In another embodiment, the composition is a pharmaceutical composition.

In one embodiment, the pharmaceutical composition comprises a pharmaceutically acceptable excipient.

In other embodiments, a pharmaceutical composition of the present invention is delivered to a subject via an oral, parenteral, enteral, or a topical route of administration, preferably oral administration. In one embodiment, the pharmaceutical composition is an oral dosage form. In various embodiments, the oral dosage form is a solid, liquid, or semi-solid oral dosage form.

Examples of parental routes include, without limitation, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrarenal, intrasinal, intraspinal, intrasynovial, intratendinous, intratesticular, intrathecal, intrathoracic, intratubular, intratympanic, intrauterine, intravascular, intravenous (bolus or infusion), intraventricular, intravesical and subcutaneous.

Enteral routes of administration include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories. In a preferred embodiment, the route of administration is oral. In a further embodiment, the oral dose is formulated as an immediate or extended-release dosage form.

The pharmaceutical composition comprises a therapeutically effective amount of at least one of the xanthophyll derivatives according to the invention and at least one pharmaceutically acceptable excipient. The choice of excipient(s) will depend on factors such as the particular mode of administration and the nature of the dosage form. Solutions or suspensions used for intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA) and diethylenetriamine pentaacetate (DTPA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A pharmaceutical formulation of the present invention may be in any pharmaceutical dosage form. The pharmaceutical formulation may be, for example, a tablet, capsule, extrudate, nanoparticulate material, e.g., granulated particulate material or a powder, a lyophilized material for reconstitution, liquid suspension, injectable suspension or solution, suppository, or topical or transdermal preparation or patch. The pharmaceutical formulations generally contain about 1% to about 99% by weight of at least one xanthophyll derivative of the invention and 99% to 1% by weight of a suitable pharmaceutical excipient. In one embodiment, the dosage form is an oral dosage form. In another embodiment, the dosage form is a parenteral dosage form. In one embodiment, the dosage form is an oral dosage form selected from a syrup, drops, solution, suspension, tablet, bolus, troche, tincture, oral/buccal/sublingual spray, lozenge, dissolving strip, or capsule. In one embodiment, the capsule is a hard gelatin capsule, a soft gelatin capsule, a starch capsule or an enteric coated capsule. In a one embodiment, the unit dose is a hard gelatin capsule. In a further embodiment, the unit dose is a soft gelatin capsule.

In another embodiment, the invention provides for a unit dose of the pharmaceutical composition of the present invention.

In the above methods of treating, the treatment is carried out by one or more unit doses administered per day. The daily dose of the pharmaceutical composition is preferably approximately 0.1-50 mg/kg body weight, 0.1-10 mg/kg body weight, 0.1-5 mg/kg body weight, 0.1-2 mg/kg body weight, 5-10 mg/kg body weight, 10-20 mg/kg body weight, 20-30 mg/kg body weight, 30-40 mg/kg body weight, 40-50 mg/kg body weight, 50-60 mg/kg body weight or 10-1000 mg, in particular 10-200 mg, 10-100 mg, 5-50 mg, 50-100 mg, 100-150 mg, 150-200 mg, 200-300 mg, 300-400 mg, 400-500 mg, 500-750 mg, 750-1000 mg, 1000-1500 mg, 1500-2000 mg, 2000-2500 mg, 2500-3000 mg, 3000-3500 mg, or 3500-4000 mg, in each case calculated as the parent xanthophyll, e.g. astaxanthin. The doses can be administered in any convenient dosing schedule to achieve the stated beneficial effects. For example, the doses can be taken 1, 2 or 3 times daily.

In one embodiment, the unit dose comprises 5-15 mg, 10-40 mg (e.g., 15-40 mg, 20-30 mg, 20-40 mg, 25-40 mg, 30-40 mg, 35-40 mg, 10-35 mg, 10-30 mg, 10-25 mg, 10-20 mg, 10-15 mg), 30-60 mg (e.g., 35-60 mg, 40-60 mg, 45-60 mg, 50-60 mg, 55-60 mg, 30-55 mg, 30-50 mg, 30-45 mg, 30-40 mg, 30-35 mg), or 10-4000 mg (10-100 mg, 10-200 mg, 5-50 mg, 50-100 mg, 100-150 mg, 150-200 mg, 200-300 mg, 300-400 mg, 400-500 mg, 500-750 mg, 750-1000 mg, 1000-1500 mg, 1500-2000 mg, 2000-2500 mg, 2500-3000 mg, 3000-3500 mg, or 3500-4000 mg).

In one embodiment, the xanthophyll derivative compositions of the present invention are food additives or feed additives. Typically, the food or feed additives comprise the xanthophyll derivative composition according to the invention and at least one ingredient which is suitable for foodstuffs or feedstuffs. An ingredient which is suitable for foodstuffs or feedstuffs is to be understood as meaning for the purposes of the invention a substance which is approved for animal and/or human nutrition. Suitable ingredients are, in particular, diluents. Diluents may included vegetable oils and organic solvents, but also solid carriers such as fats, fatty acids, waxes, fatty alcohols and fatty acid esters of fatty alcohols, carbohydrates, sugar alcohols and inorganic fillers which are approved for the manufacture of foodstuffs or feedstuffs.

Examples of solvents include: C₁-C₆-alkanols such as, for example, ethanol, 1-propanol, 2-propanol, 1-butanol, 2-butanol, 2-methyl-1-propanol, 3-methyl-1-butanol, 1-pentanol and their mixtures; C₁-C₄-alkyl esters of aliphatic C₁-C₄-carboxylic acids such as, for example, the methyl, ethyl, n-propyl, isopropyl, n-butyl, 2-butyl or isobutyl esters of formic acid, acetic acid, propionic acid or butyric acid, such as methyl acetate, ethyl acetate, n-propyl acetate, isopropyl acetate, n-butyl acetate, isobutyl acetate, ethyl formate and their mixtures; and aliphatic, in particular noncyclic, ketones having 3 to 6 C atoms such as acetone, methyl ethyl ketone, isobutyl methyl ketone and their mixtures; and mixtures of the abovementioned solvents from various classes of the abovementioned solvents.

Suitable carbohydrates include mono-, di-, oligo- and polysaccharides. Examples of monosaccharides and disaccharides are mainly glucose, fructose, galactose, mannose, maltose, sucrose and lactose. Suitable polysaccharides are starch and oligomeric starch degradation products (dextrins) and cellulose powder.

Suitable sugar alcohols include sorbitan and glycerol.

Suitable fats and oils may be of synthetic, mineral, vegetable or animal origin. Examples of oils are vegetable oils such as soya oil, sunflower oil, corn oil, linseed oil, rapeseed oil, safflower oil, wheat germ oil, rice oil, coconut oil, almond oil, apricot kernel oil, palm oil, palm kernel oil, avocado oil, jojoba oil, hazelnut oil, walnut oil, peanut oil, pistachio oil, triglycerides of medium-chain (═C₈-C₁₀) vegetable fatty acids (so-called MCT oils) and PUFA oils (PUFA=polyunsaturated fatty acids such as eicosapentaenoic acid (EPA), docosahexaenoic acid (DHA) and α-linolenic acid), furthermore semisynthetic triglycerides, for example caprylic/capric acid triglycerides such as the miglyol types, furthermore liquid paraffin, liquid hydrogenated polyisobutenes, squalane, squalene, furthermore animal oils and fats such as fish oils including mackerel oil, sprat oil, tuna oil, halibut oil, cod oil and salmon oil. Preferred are vegetable oils and oils of animal origin which are liquid at 40° C., in particular vegetable oils such as soya oil, sunflower oil, safflower oil, corn oil, olive oil, linseed oil, rapeseed oil, rice oil, coconut oil, peanut oil, palm oil, palm kernel oil, PUFA oils, MCT oils, furthermore fish oils, and mixtures of these oils.

In contrast to oils, fats usually have a melting point of above 30° C. Examples of fats include: saturated fatty acids having 12 to 30 C atoms (saturated C₁₂-C₃₀-fatty acids), in particular 14 to 28 C atoms (saturated C₁₄-C₂₈-fatty acids) and specifically 16 to 24 C atoms (saturated C₁₆-C₂₄-fatty acids) such as myristic acid, palmitic acid, margaric acid, stearic acid, arachic acid, behenic acid, cerotic acid, melissic acid and lignoceric acid, and their mixtures; fatty acid esters of saturated fatty acids having 14 to 30 C atoms, in particular 14 to 28 C atoms and specifically 16 to 24 C atoms, such as the esters of palmitic acid, margaric acid, stearic acid, arachic acid, behenic acid, cerotic acid, melissic acid and lignoceric acid and their mixtures, in particular the mono-, di- and triglycerides of the abovementioned fatty acids and their mixtures (hereinbelow referred to as fatty acid glycerides), in particular of the abovementioned saturated fatty acids and specifically those saturated fatty acids which have 14 to 28 C atoms and specifically 16 to 28 C atoms, and esters of the abovementioned saturated fatty acids having preferably 14 to 28 C atoms and specifically 16 to 28 C atoms, with C₁₀-C₃₀-fatty alcohols, in particular with C₁₄-C₂₈-fatty alcohols. The abovementioned esters of saturated fatty acids may also comprise mono- or polyunsaturated fatty acids in esterified form in an amount of up to 10% by weight, based on the fatty acid content in the ester. In particular, the content of unsaturated fatty acid components in these esters amounts to less than 5% by weight, based on the total fatty acid components in the ester.

The oils and fats may be refined oils or crude oils/fats which still comprise origin-specific impurities such as proteins, phosphate, alkali metal salts, alkaline earth metal salts and the like in usual amounts.

Suitable fatty alcohols are in particular saturated aliphatic alcohols having 8 to 30 C atoms (hereinbelow also C₈-C₃₀-fatty alcohols), such as, for example, cetyl alcohol, stearyl alcohol, nonadecanol, arachidyl alcohol, behenyl alcohol, lignoceryl alcohol, ceryl alcohol, myricyl alcohol and melissyl alcohol.

Suitable waxes are in particular natural waxes of vegetable or animal origin such as beeswax, candelilla wax, shellac wax, shea butter and carnauba wax, carbohydrate waxes such as paraffin waxes, ceresin, Sasol waxes, ozokerite and microwaxes.

Examples of inert inorganic fillers which are suitable for foodstuffs are inorganic materials in pulverulent form (inorganic fillers), for example oxides such as aluminum oxide, silica, titanium dioxide, silicates such as sodium silicate, magnesium silicate, talc, calcium silicate, zinc silicate, aluminum silicates such as sodium aluminum silicate, potassium aluminum silicate, calcium aluminum silicate, bentonite, kaolin and sodium chloride.

The adjuvants which are suitable for foodstuffs and feedstuffs furthermore include dispersants, including lipophilic dispersants for dispersing the xanthophyll compositions in lipophilic carriers and protective colloids for dispersing the xanthophyll compositions according to the invention in hydrophilic carriers such as water, furthermore antioxidants (oxidation stabilizers), and colorants which are approved for foodstuffs.

Examples of antioxidants are tocopherols such as α-tocopherol, α-tocopherol palmitate, α-tocopherol acetate, tert-butyl hydroxytoluene, tert-butyl hydroxyanisole, propyl gallate, pyrogallol, ascorbic acid, its salts and esters such as, for example, sodium ascorbate, calcium ascorbate, ascorbyl phosphate ester and ascorbyl palmitate and ethoxyquin. If desired, the antioxidants are typically present in the additives according to the invention in amounts of from 0.01 to 10% by weight, based on the total weight of the additive.

Examples of preservatives include benzoic acid and its salts, in particular its sodium, potassium and calcium salts, 4-hydroxybenzoic acid (PHB) and its salts, in particular its sodium, potassium and calcium salts, the salts of the PHB alkyl esters, such as the sodium salt of the PHB methyl ester, the sodium salt of the PHB ethyl ester and the sodium salt of the PHB propyl ester, sorbic acid and its salts, in particular its sodium, potassium and calcium salts, salts of propionic acid such as, in particular, its sodium, potassium and calcium salts, boric acid and lactic acid and their salts. If desired, preservatives are typically present in the additives in amounts of from 0.001 to 2% by weight, based on the total weight of the additives.

Typical lipophilic dispersants are ascorbyl palmitate, polyglycerol fatty acid esters such as polyglycerol-3 polyricinoleate (PGPR90), sorbitan fatty acid esters, in particular sorbitan C₁₀-C₂₈-fatty acid esters such as, for example, mono- and di-C₁₀-C₂₈-fatty acid esters of sorbitan, such as sorbitan monolaurate (SPAN20), sorbitan monooleate (SPAN80) and sorbitan monostearate (SPAN60), ethoxylated sorbitan fatty acid esters such as PEG(20) sorbitol monooleate, monoesters of lactic acid with saturated C₁₀-C₂₄-fatty acids, sugar esters of saturated C₁₆-C₂₈-fatty acids, propylene glycol fatty acid esters, and phospholipids such as lecithin. If desired, lipophilic dispersants are typically present in the additives in amounts of from 0.01 to 10% by weight, based on the total weight of the additives.

Protective colloids are polymeric substances which are suitable for dispersing water-insoluble solids in aqueous compositions, Suitable protective colloids which are approved for foodstuffs and feedstuffs comprise, for example, proteinaceous protective colloids and lignosulfonic acids and salts thereof, oligo- and polysaccharides, including modified polysaccharides, such as, for example, dextrins, cellulose and cellulose derivatives such as methylcellulose, carboxymethylcellulose and their salts, hydroxyethylcellulose, hydroxypropylcellulose and hydroxypropylmethylcellulose, gum arabic, pectins, polyvinyl alcohol, including partially hydrolyzed polyvinyl alcohol, polyvinylpyrrolidone and mixtures of the abovementioned protective colloids.

### Methods of Treating

In addition to promoting general state of health, liver health, cardiovascular health or improving inflammatory health the compositions of the present invention may be used to treat a variety of diseases and conditions. Accordingly, another aspect provides for a method of treating a subject having a disease or disorder that would benefit from the administration of a xanthophyll derivative of the present invention, said method comprising the step of administering to said subject an effective amount of said xanthophyll derivative. The xanthophyll derivatives of the present invention are therapeutically useful for the treatment and/or prevention of a disease for which it is indicated, e.g., fibrotic diseases such as NASH. Accordingly, in another aspect, the invention also relates to: methods of treating a subject using a xanthophyll derivative of the present invention or salt or co-crystal thereof; methods of treating a subject using a pharmaceutical composition comprising the xanthophyll derivative or salt or co-crystal thereof and a pharmaceutically acceptable excipient; and use of the xanthophyll derivative or xanthophyll derivative pharmaceutical composition as a medicament for the treatment of a disease or condition as described herein.

NAFLD includes two pathologically distinct conditions with different prognoses: non-alcoholic fatty liver (NAFL) and non-alcoholic steatohepatitis (NASH); the latter covers a spectrum of disease severity, including fibrosis, cirrhosis and hepatocellular carcinoma (HCC). Diagnostic and guidelines for the management of NAFLD are disclosed in “European Association for the Study of the Liver (EASL) et al: EASL-EASD-EASO clinical practice guidelines for the management of non-alcoholic fatty liver disease.”J Hepatol. 64(6):1388-402, 2016.

NAFLD is characterized by excessive hepatic fat accumulation, associated with insulin resistance (IR), and defined by the presence of steatosis in >5% of hepatocytes according to histological analysis or by a proton density fat fraction (providing a rough estimation of the volume fraction of fatty material in the liver)>5.6% assessed by proton magnetic resonance spectroscopy (1H-MRS) or quantitative fat/water selective magnetic resonance imaging (MRI). The diagnosis of NAFLD requires the exclusion of both secondary causes and of a daily alcohol consumption≥30 g for men and ≥20 g for women. The definitive diagnosis of NASH requires a liver biopsy.

NAFL is hepatic steatosis without significant inflammation or fibrosis. Liver biopsy is essential for the diagnosis of NASH and is the only procedure that reliably differentiates NAFL from NASH, despite limitations due to sampling variability. NAFL encompasses: a) steatosis alone, b) steatosis with lobular or portal inflammation, without hepatocyte ballooning (cell swelling and enlargement), or c) steatosis with hepatocyte ballooning but without inflammation. The diagnosis of NASH requires the joint presence of steatosis, hepatocyte ballooning and lobular inflammation; pericellular fibrosis is often present. NASH is classified by degree of fibrosis: F0—no fibrosis; F1—mild fibrosis; F2—significant fibrosis; F3—advanced or bridging fibrosis; F4—severe fibrosis (cirrhosis).

In one embodiment, the invention provides for a method of treating nonalcoholic fatty liver disease (NAFLD) in a subject, said method comprising the step of administering to said subject a therapeutically effective amount of a xanthophyll derivative of the present invention.

In one embodiment, the invention provides for a method of treating non-alcoholic fatty liver (NAFL) in a subject, said method comprising the step of administering to said subject a therapeutically effective amount of a xanthophyll derivative of the present invention.

In one embodiment, the invention provides for a method of treating non-alcoholic steatohepatitis (NASH) in a subject, said method comprising the step of administering to said subject a therapeutically effective amount of a xanthophyll derivative of the present invention. In a further embodiment, said NASH is selected from the group consisting of: NASH stage F0, NASH stage F1, NASH stage F2, NASH stage F3 and NASH stage 4.

In some embodiments the present invention relates to a xanthophyll derivative for use in the treatment of NASH, wherein said use reduces the relative liver weight, plasma alanine aminotransferase levels, liver triglyceride content and/or liver cholesterol. The relative liver weight is defined as liver weight as percentage of total body weight.

In some embodiments said subject is overweight or obese or suffers from hyperglycemia, type 2 diabetes, impaired glucose tolerance or type 1 diabetes.

BMI (body mass index) is a measure of body fat based on height and weight. The formula for calculation is BMI=(weight in kilograms)/(height in meters)2. In some embodiments the present invention relates to a compound use in the treatment of NASH, wherein said compound is administered in a therapeutically effective amount to a subject in need thereof, wherein said subject has a BMI of at least 25 kg/m2. In some embodiments said subject has a BMI of at least 30 kg/m2. In some embodiments said subject has a BMI between 30-50 kg/m2.

In one embodiment, the invention provides for a method for the treatment of lobular inflammation in a subject, said method comprising the step of administering to said subject a therapeutically effective amount of a xanthophyll derivative of the present invention.

In one embodiment, the invention provides for a method for the treatment of portal inflammation in a subject, said method comprising the step of administering to said subject a therapeutically effective amount of a xanthophyll derivative of the present invention.

In one embodiment, the invention provides for a method for the treatment of hepatocyte ballooning in a subject, said method comprising the step of administering to said subject a therapeutically effective amount of a xanthophyll derivative of the present invention.

In one embodiment, the invention provides for a method for the treatment of low-density lipoprotein (LDL) oxidation in a subject, said method comprising the step of administering to said subject a therapeutically effective amount of a xanthophyll derivative of the present invention.

In one embodiment, the invention provides for a method for the treatment of non-alcoholic steatohepatitis-derived hepatocellular carcinoma in a subject, said method comprising the step of administering to said subject a therapeutically effective amount of a xanthophyll derivative of the present invention.

In one embodiment, the invention provides for a method for the treatment of an alcohol related liver injury selected from: alcohol-induced liver inflammation and oxidative stress or alcoholic liver disease (ALD) in a subject, said method comprising the step of administering to said subject a therapeutically effective amount of a xanthophyll derivative of the present invention. The histological diagnoses in ALD include alcoholic fatty liver (AFL), alcoholic steatohepatitis (ASH), alcoholic steatohepatitis-associated fibrosis, alcoholic cirrhosis or HCC. In one embodiment, the invention provides for a method for the treatment of an alcoholic liver disease selected from: alcoholic fatty liver (AFL), alcoholic steatohepatitis (ASH), alcoholic steatohepatitis-associated fibrosis, alcoholic cirrhosis or HCC in a subject, said method comprising the step of administering to said subject a therapeutically effective amount of a xanthophyll derivative of the present invention. In one embodiment, the invention provides for a method of treating alcohol-induced inflammation and oxidative stress in macrophages in a subject, said method comprising the step of administering to said subject a therapeutically effective amount of a xanthophyll derivative of the present invention

Fibrosis is characterized by excessive production and deposition of extracellular matrix proteins with a detrimental impact on organ function. In one embodiment, the present invention provides a method of treating fibrosis in a subject comprising administering to the subject an effective amount of a xanthophyll derivative of the present invention. In a particular embodiment, the subject is a human. In one embodiment, the fibrotic disease is selected from: acute interstitial pneumonitis, arthrofibrosis, fibrosis in asthma (airway remodeling), atherosclerosis, bone-marrow fibrosis, cardiac fibrosis, renal fibrosis, cirrhosis of gallbladder, NASH stage F1, NASH stage F2, NASH stage F3 and NASH stage 4, primary sclerosing cholangitis, primary biliary cholangitis (primary biliary cirrhosis), cholangiocarcinoma, alcoholic liver disease, alcoholic fatty liver (AFL), alcoholic steatohepatitis (ASH), alcoholic steatohepatitis-associated fibrosis, alcoholic cirrhosis, drug-induced liver disease, hemochromatosis, auto-immune hepatitis, chronic viral hepatitis B or C, keloid scar, hypertrophic scar, cryptogenic organizing pneumonia, cystic fibrosis, desquamative interstitial pneumonia, diffuse parenchymal lung disease, Dupuytren's contracture, endomyocardial fibrosis, fibrosis as a result of Graft-Versus-Host Disease (GVHD), idiopathic pulmonary fibrosis, idiopathic interstitial fibrosis, interstitial lung disease, interstitial pneumonitis, lymphocytic interstitial pneumonia, multifocal fibrosclerosis, muscle fibrosis, myelofibrosis, nephrogenic systemic fibrosis, nonspecific interstitial pneumonia, organ transplant fibrosis, pancreatic fibrosis, Wilson's disease, Peyronie's disease, renal fibrosis, respiratory bronchiolitis, retroperitoneal fibrosis, scarring after surgery, scleroderma (circumscribed morphea, generalized morphea and linear scleroderma), systemic scleroderma (limited, diffuse and sine), subepithelial fibrosis, or uterine fibrosis. In one embodiment, the xanthophyll derivative reduces the level of fibrogenesis by at least 80%, at least 70%, at least 60%, at least 50%, at least 40%, at least 30%, at least 20%, at least 10%, at least 5% or at least 2%. In one embodiment, the present invention provides methods for reversing fibrosis, e.g., liver fibrosis, in a subject, said method comprising the step of administering to said subject a therapeutically effective amount of a xanthophyll derivative of the present invention. In some embodiments, the fibrosis is a fibrosis-related liver disease is selected from: NAFLD, AFL, NASH, ASH, non-alcoholic steatohepatitis associated fibrosis, alcoholic steatohepatitis associated fibrosis, non-alcoholic cirrhosis (e.g., primary biliary cirrhosis), alcoholic cirrhosis, Hepatitis B, Hepatitis C, Wilson's disease, hemochromatosis, or biliary obstruction.

In one embodiment, the invention provides for a method of reverting activated hepatic stellate cells (aHSCs) in a subject to an inactivated HSCs (iHSCs) phenotype, said method comprising the step of administering to said subject a therapeutically effective amount of a xanthophyll derivative of the present invention.

In one embodiment, the invention provides for a method of reverting activated hepatic stellate cells (aHSCs) in a subject to a quiescence HSCs (qHSCs) phenotype, said method comprising the step of administering to said subject a therapeutically effective amount of a xanthophyll derivative of the present invention.

In one embodiment, the invention provides for a method of inhibiting the activation of quiescence HSCs (qHSCs) to activated hepatic stellate cells (aHSCs) in a subject, said method comprising the step of administering to said subject a therapeutically effective amount of a xanthophyll derivative of the present invention.

In one embodiment, the invention provides for a method of inhibiting the activation of inactive HSCs (iHSCs) to activated hepatic stellate cells (aHSCs) in a subject, said method comprising the step of administering to said subject a therapeutically effective amount of a xanthophyll derivative of the present invention.

In one embodiment, the invention provides for a method of inhibiting TGFβ1 signaling in HSCs in a subject, said method comprising the step of administering to said subject in need an effective amount of a xanthophyll derivative.

In one embodiment, the invention provides for a method of inhibiting HDAC₉ expression in HSCs in a subject, said method comprising the step of administering to said subject a therapeutically effective amount of a xanthophyll derivative of the present invention.

In one embodiment, the invention provides for a method of inhibiting cellular reactive oxygen species (ROS) accumulation in a subject, said method comprising the step of administering to said subject a therapeutically effective amount of a xanthophyll derivative of the present invention.

In one embodiment, the invention provides for a method of inhibiting cellular reactive oxygen species (ROS) accumulation in HSCs in a subject, said method comprising the step of administering to said subject a therapeutically effective amount of a xanthophyll derivative of the present invention.

In one embodiment, the invention provides for a method of inhibiting expression of myocyte enhancer factor 2 (MEF2) in HSCs in a subject, said method comprising the step of administering to said subject a therapeutically effective amount of a xanthophyll derivative of the present invention.

In one embodiment, the invention provides for a method of inhibiting basal expression of fibrogenic genes in HSCs in a subject, said method comprising the step of administering to said subject a therapeutically effective amount of a xanthophyll derivative of the present invention.

Primary biliary cholangitis (PBC), also as primary biliary cirrhosis, is an autoimmune disease of the liver resulting from a slow, progressive destruction of the small bile ducts of the liver, causing bile and other toxins to build up in the liver, a condition called cholestasis. Further slow damage to the liver tissue can lead to scarring, fibrosis, and eventually cirrhosis. PBC mainly affects the small bile ducts in the liver itself and is not associated with changes in the large bile ducts of the liver. Currently, a diagnosis of PBC is made with confidence on a combination of abnormal serum liver tests (elevation of alkaline phosphatase (AP) of liver origin for at least 6 months) and presence of AMA (≥1:40) in serum. The diagnosis is confirmed by disclosing characteristic histological features of florid bile duct lesions. AMA-positive individuals with normal AP carry a high risk to develop PBC during follow-up. A liver biopsy is needed for the diagnosis of PBC in the absence of PBC specific antibodies.

There are four stages of PBC:
Stage 1 (Portal Stage): Normal sized triads; portal inflammation, subtle bile duct damage. Granulomas — nodules filled with a variety of inflammatory cells—are often detected in this stage.
Stage 2 (Periportal Stage): Enlarged triads; periportal fibrosis and/or inflammation. Typically characterized by the finding of a proliferation of small bile ducts.
Stage 3 (Septal Stage): Active and/or passive fibrous septae.
Stage 4 (Biliary Cirrhosis): Nodules present; garland or jigsaw pattern.

(See, Scheuer P. J. Proc Roy Soc Med 1967; 60:1257-1260; Ludwig J., et al. Virchows Arch A Pathol Anat Histol 1978; 379:103-112).

In one embodiment, the invention provides for a method for the treatment of primary biliary cholangitis in a subject, said method comprising the step of administering to said subject a therapeutically effective amount of a xanthophyll derivative of the present invention. In a further embodiment, the primary biliary cholangitis is selected from: stage 1, stage 2, stage 3 or stage 4 primary biliary cholangitis.

Primary sclerosing cholangitis (PSC) occurs because of inflammation in the bile ducts (cholangitis), which results in hardening (sclerosis) and narrowing of the ducts. A key feature of PSC is the development of scar tissue (fibrosis) that predominantly affects the medium- to large-sized bile ducts within and outside the liver. One main staging system for PSC has been devised. Ludwig et al. (above) described four stages of PSC:
Stage 1 (Cholangitis or portal hepatitis): Infiltration of the bile duct by lymphocytes with degeneration of the epithelial cells of the bile duct. These findings are not present outside the portal triads.
Stage 2 (periportal fibrosis or periportal hepatitis): There is more widespread involvement with fibrosis, inflammation infiltration in the periportal parenchyma with piecemeal necrosis of the periportal hepatocytes. The portal triads are enlarged but there is relative absence of bile ducts (bile ductopenia).
Stage 3 (septal fibrosis, bridging necrosis or both): There are portal-to-portal fibrous bridges with severe degeneration of the ducts and ductopenia.
Stage 4 (Biliary cirrhosis): End stage liver disease with frank cirrhosis.

In one embodiment, the invention provides for a method for the treatment of primary sclerosing cholangitis in a subject, said method comprising the step of administering to said subject a therapeutically effective amount of a xanthophyll derivative of the present invention. In a further embodiment, the primary biliary cholangitis is selected from: stage 1, stage 2, stage 3 or stage 4 primary sclerosing cholangitis.

In one embodiment, the invention provides for a method of treating a cardiometabolic disorder/metabolic syndrome, said method comprising the step of administering to said subject a therapeutically effective amount of a xanthophyll derivative of the present invention. In further embodiments, the cardiometabolic disorder/metabolic syndrome is selected from: acute coronary syndrome, acute myocardial infarction, angina pectoris, aortic and mitral valve disorders, arrhythmia/atrial fibrillation, arterial occlusive diseases, atherosclerosis, cardiomyopathy, carotid atherosclerosis, cerebral atherosclerosis, chronic kidney disease, coagulopathies leading to thrombus formation in a vessel, coronary heart disease, diabetic autonomic neuropathy, diabetic nephropathy, dyslipidemia, endocarditis, high LDL levels, hypercholesterolemia, hypercholesterolemia in HIV infection, hyperlipidemia, hyperlipoproteinemia, hyperphosphatemia, hypertension, hypertriglyceridemia, impaired glucose tolerance, insulin resistance, intermittent claudication, Kawasaki disease, low HDL levels, myocardial ischemia, omega-3 deficiency, orthostatic hypotension, peripheral arterial disease, phospholipid deficiency, pulmonary or venous embolism, secondary prevention of myocardial infarction, shock, stroke, type 2 diabetes or valvular heart disease.

In one embodiment, the invention provides for a method of treating a cognitive disease or disorder, said method comprising the step of administering to said subject a therapeutically effective amount of a xanthophyll derivative of the present invention. In further embodiments, the cognitive disease or disorder is selected from: Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease), mood deterioration, age-related cognitive decline, or concentration and attention impairment.

In one embodiment, the invention provides for a method of treating a neurodegenerative/neuroinflammatory disease, said method comprising the step of administering to said subject a therapeutically effective amount of a xanthophyll derivative of the present invention. In further embodiments, the neurodegenerative/neuroinflammatory disease is selected from: Parkinson's, Alzheimer's, multiple sclerosis, frontotemporal dementia (Pick's disease), Huntington's disease, Lewy body dementia, Friederichs's ataxia, multiple system atrophy and amyotrophic lateral sclerosis.

In one embodiment, the invention provides for a method of treating an inflammation or an inflammatory disease or disorder, said method comprising the step of administering to said subject a therapeutically effective amount of a xanthophyll derivative of the present invention. In further embodiments, the inflammation or an inflammatory disease or disorder is selected from: organ transplant rejection; reperfusion injury (ischemia-reperfusion injury (IRI) or reoxygenation injury); chronic inflammatory diseases of the joints, including arthritis, rheumatoid arthritis, osteoarthritis, polyarticular or pauciarticular juvenile rheumatoid arthritis; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis or eczema. The inflammatory disease may also be a systemic inflammation of the body, exemplified by gram-positive or gram-negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In one embodiment, the invention provides for a method of treating a cancer, said method comprising the step of administering to said subject a therapeutically effective amount of a xanthophyll derivative of the present invention. In further embodiments, the cancer is selected from: cervical carcinoma, bladder carcinoma, mammary carcinoma, lung carcinoma, astrocytoma, oligodendroglioma, glioblastoma, anaplastic astrocytoma, and anaplastic oligodendroglioma.

Another aspect provides for a method of making a composition of the present invention.

In one embodiment, the invention provides for a method of making a composition of the present invention, said method comprising the steps of:
providing one or more active ingredients, wherein at least one active ingredient is at least one xanthophyll derivative, and at least one excipient; and
combining said at least one active ingredient, including at least one xanthophyll derivative, and said at least one excipient to form a mixture. In one embodiment, the mixture is an isotropic or homogeneous mixture.

The invention further provides for a method: for increasing at least one parameter, as compared to the parent xanthophyll, selected from the group consisting of stability, solubility, dissolution, oral bioavailability, Cmax, absorption, onset of action; or for decreasing time to Tmax or intra-subject variability; comprising the steps of:
providing one or more active ingredients, wherein at least one active ingredient is an xanthophyll derivative, and at least one excipient; and
combining said one or more active ingredients and said at least one excipient to form a mixture. In one embodiment, the mixture is an isotropic or homogeneous mixture.

The starting xanthophyll compounds of the present invention may be obtained commercially, chemically synthesized (e.g., EP 3065568 A1, EP 1197483, EP 1285912, US 2018/0055788 A1) by a conventional chemical synthesis method, or extracted and purified from a microorganism, an animal, a plant, or the like. Microorganism, animal or plant-derived product may be obtained from a suitable microorganism, animal or plant, e.g., the freshwater microalgae Haematococcus pluvialis or the basidiomycetous yeast Xanthophyllomyces dendrorhous (anamorph Phaffia rhodozyma). See, e.g., WO 2018/056160, WO 2018/043147 A1.

The xanthophyll derivatives of the present invention can be made to a pharmaceutically acceptable ester or amide in accordance with an ordinary method, for example, by a condensation reaction of a carboxylic acid of the derivative or prodrug moiety with an alcohol on the xanthophyll.

### EXAMPLES

### Compound Synthesis

The xanthophyll, e.g., astaxanthin, derivatives of the present invention can be manufactured in principle according to synthetic methods known per se for esterification, according to the nature of the group R1 (See e.g., US20080008798, US20170081289 and U.S. Pat. No. 7,446,107 incorporated by reference in their entireties). For example, the xanthophyll, e.g., astaxanthin, is reacted with the pertinent acid RCOOH as such or as its acid chloride RCOCl or acid anhydride (RCO)₂O. These processes for producing the xanthophyll, e.g., astaxanthin derivatives of the formulas XVI-XXX represent a further aspect of the present invention.

In the case of esterification with an acid chloride or acid anhydride, the reaction is generally conducted in an inert solvent and in the presence of an organic base. The solvent to be used is not particularly limited, as long as it is inert to the present reaction, but it may be, for example, an aliphatic hydrocarbon such as hexane, heptane, ligroin and petroleum ether; an aromatic hydrocarbon such as benzene, toluene and xylene; a halogenated hydrocarbon such as methylene chloride, chloroform, 1,2-dichloroethane and carbon tetrachloride; an ether such as diethyl ether, di-isopropyl ether, tetrahydrofuran, dioxane, dimethoxy-ethane and diethylene glycol dimethyl ether; a ketone such as acetone; an amide such as formamide, dimethylformamide, dimethylacetoamide and hexamethylphosphoric acid triamide; a sulfoxide such as dimethyl sulfoxide; or sulfolane, and it is preferably a halogenated hydrocarbon, an ether or an amide and most preferably methylene chloride, chloroform, tetrahydrofuran, dioxane or dimethylformamide. The solvents can be used alone or as a combination.

The base to be used is, for example, an alkali metal carbonic acid salt such as lithium carbonate, sodium carbonate and potassium carbonate; an alkali metal hydrogen carbonic acid salt such as lithium hydrogen carbonate, sodium hydrogen carbonate and potassium hydrogen carbonate; an alkali metal hydride such as lithium hydride, sodium hydride and potassium hydride; an alkali metal hydroxide such as lithium hydroxide, sodium hydroxide and potassium hydroxide; an alkali metal alkoxide such as lithium methoxide, sodium methoxide, sodium ethoxide and potassium t-butoxide; or an organic amine such as triethyl-amine, tributylamine, N,N-diisopropylethylamine, N-methylmorpholine, pyridine, 4-(N,N-dimethylamino)pyridine, N,N-dimethylaniline, N,N-diethylaniline, 1,5-diazabicyclo [4.3.0]non-5-ene, I,4-diazabicyclo[2.2.2]octane (DABCO) and I,8-diazabicyclo[5,4.0]-7-undecene (DBU) and it is preferably an organic amine and most preferably triethylamine, 4-(N,N-dimethylamino)pyridine or N,N-diisopropylethylamine. The bases can be used alone or as a combination.

The molar ratio of astaxanthin:acid chloride or acid anhydride:base is conveniently in the range of 1:2-6:2-10. It has been found to be advantageous to conduct the esterification under an inert atmosphere, preferably using nitrogen or argon as the inert gas.

Where the acid itself is used to esterify the astaxanthin, the conditions are generally similar to those employed for esterifications with an acid chloride or anhydride in respect of the solvent/dispersion medium and reaction temperatures. However, an active esterifying agent is generally employed in combination or not with a base. The active esterifying agent to be used may be, for example, an N-hydroxy compound such as N-hydroxysuccinimide, 1-hydroxybenzotriazole and N-hydroxy-5-norbornen-2,3-dicarboxyimide; a disulfide compound such as dipyridyl disulfide; a carbodiimide such as N,N′-diisopropylcarbodiimide, dicyclohexylcarbodiimide, 1-ethyl-3-(3-di-methylaminopropyl)carbodiimide hydrochloride and bis-(trimethylsilyl)carbodiimide; 1,1′-carbonylbis-1H-imidazole; 4-(4,6-dimethoxy-1,3,5-triazin-2-yl)-4-methylmorpholinium chloride (DMTMM), diphenylphosphoric acid azide, hexafluorophosphoric acid benzotriazol-1-yloxy-tris(dimethylamino)phosphonium or triphenylphosphine and it is preferably N,N′-diisopropylcarbodiimide, dicyclohexylcarbodiimide, 1-ethyl-3-(3-dimethylaminopropyl)carbodiimide hydrochloride, 4-(4,6-dimethoxy-1,3,5-triazin-2-yl)-4-methylmorpholinium chloride (DMTMM), diphenyl phosphoric acid azide or 1,1′-carbo-nylbis-1H-imidazole and most preferably N,N′-diisopropylcarbodiimide, 1-ethyl-3-(3-dimethylaminopropyl)-carbodiimide hydrochloride, 4-(4,6-dimethoxy-1,3,5-triazin-2-yl)-4-methylmorpholinium chloride (DMTMM) or 1,1′-carbonylbis-1H-imidazole.]

The molar ratio of astaxanthin:carboxylic acid-active esterifying agent is conveniently in the range of 1:2-6:2-7.

In all these cases the product, i.e., the astaxanthin derivative of formula I, can be isolated and purified by methods known per se, e.g., by adding a solvent to induce the separation of the crude product from the mixture after reaction, and crystallization of the collected crude product. Column chromatography may further be employed in purification.

The pertinent acids RCOOH, acid chlorides RCOCI and acid anhydrides, (RCO)₂O, used as starting materials in the above-described processes for producing the astaxanthin derivatives of the formula XVI-XXX are either known compounds, or can be readily produced by processes analogous to the processes for producing the related known starting materials.

### Example 1. Synthesis of CCXX, (all-E)-3S,3′S-Astaxanthin-di-N-nicotinoglycinate, ((1S)-methyl-2-oxo-4-[(1E,3E,5E,7E,9E,11E,13E,15E,17E)-3,7,12,16-tetramethyl-18-[(4S)-2,6,6-trimethyl-3-oxo-4-({2-[(pyridin-3-yl)formamido]acetyl}oxy)cyclohex-1-en-1-yl]octadeca-1,3,5,7,9,11,13,15,17-nonaen-1-yl]cyclohex-3-en-1-yl2-[(pyridin-3-yl)formamido]acetate)

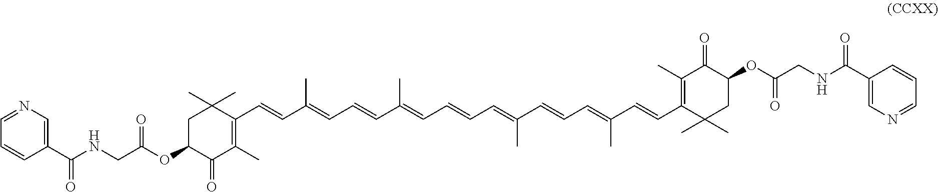 [SMILES: CC1=C(/C=C/C(C)=C/C=C/C(C)=C/C=C/C=C(C)/C=C/C=C(C)/C=C/C2=C(C)C(=O)[C@@H](OC(=O)CNC(=O)c3cccnc3)CC2(C)C)C(C)(C)C[C@H](OC(=O)CNC(=O)c2cccnc2)C1=O]

Crude free astaxanthin was obtained by de-esterification of astaxanthin oleoresin extracted from Haematococcus pluvialis algal biomass containing \tilde{ }10 w/w total astaxanthin as free, mono-esterified and di-esterified predominantly as the (all-E)-3S,3′S isomer. A 1 L one-necked round bottom flask was charged with 26 g of oleoresin and 740 mL methanol/dichloromethane 89:11 v/v at 4° C. under nitrogen atmosphere. 0.4 mL of a methanolic solution of BHT 50 mg/mL was added. 16.6 mL of a methanolic solution of potassium hydroxide 1 M was added slowly at 4° C. The reaction was monitored by silica gel TLC using as mobile phase (80/20 v/v) hexane/methanol and HPLC. After 81 hours most of the astaxanthin diesters converted to free astaxanthin. Then, 8.5 mL of a methanolic solution of acetic acid 10 mmol/mL was added until the mixture reached pH 5. The reaction solution was concentrated to half its volume in a rotary evaporator (35° C., 250 mbar vacuum). 485 mL of distilled hexane and 10 mL of water were added to reach a hexane/methanol ratio 60/40 v/v and it was stored 20 hours at −20° C. The precipitated solid was filtered and extracted with 1 L of dichloromethane/water (50/50 v/v). After separation, the dichloromethane phase was dried with anhydrous magnesium sulfate and filtered. The solvent was evaporated using a rotary evaporator. 2.51 g of an oily dark red solid was obtained. The content of free astaxanthin, predominantly as the (all-E)-3S,3′S isomer, in this final de-esterification crude was 1.28 g (55% w/w) estimated from 1H NMR data.

\tilde{ }200 mg (0.34 mmol) of free (all-E)-3S,3′S-astaxanthin (390 mg of final de-esterification crude), 462 mg (2.56 mmol) nicotinuric acid and 7 mL of dichloromethane were added to a 50 mL one-necked round bottom flask under nitrogen atmosphere. The dark red solution was stirred and cooled in an ice bath. 10 minutes after, 1217 mg (6.35 mmol) of 1-Ethyl-3-(3-dimethylaminopropyl) carbodiimide and 844 mg (6.91 mmol) of 4-Dimethylaminopyridine was added. The reaction was allowed to reach room temperature and the mixture was stirred for 24 hours until the free astaxanthin was consumed. The reaction was monitored by silica gel TLC (mobile phase (29/48/20/3, v/v/v/v) hexane/ethyl acetate/dichloromethane/acetic acid). The reaction solution was extracted with 0.25M citric acid solution (4×15 mL) and water (3×30 mL). The dichloromethane layer was dried over anhydrous magnesium sulfate. The solvent then was removed under reduced pressure via rotary evaporation. The residue was washed with distilled hexane (2×2 mL), and then dissolved in 5 mL of methanol/hexane 80/20 v/v. Hexane was added to reach an hexane/methanol ratio 60/40 V/V and the methanol layer was extracted with hexane (4×3 mL). The methanol phase was concentrated under reduced pressure by rotary evaporation and the residue was purified by preparative HPLC. The chromatographic conditions were mobile phase acetonitrile/water 95/5, flow 15 mL/min, column C₁₈ Shimpack 250×20 mm, 10 mm. 120.4 mg (0.128 mmol) of BPH-555 was obtained, 37% yield.

¹H NMR (CDCl₃, 200 MHz): δ 1.26 (s, 6H), 1.36 (s, 6H), 1.99 (s, 12H), 1.90 (s, 6H), 2.08 (m, 2H), 2.17 (m, 2H), 4.31 (dd, 2H, J=4.8; 18.2 Hz), 4.51 (dd, 2H, J=5.7; 18.2 Hz), 5.61 (dd, 2H, J=7.1 Hz), 6.14 (m, 2H), 6.29 (m, 2H), 6.31 (m, 2H), 6.41 (m, 2H), 6.46 (m, 2H), 7.39 (m, 2H, J=4.6; 7.1 Hz), 8.18 (dd, 2H, J=7.4 Hz), 8.72 (d, 2H, J=4.5 Hz), 9.09 (bs, 2H).

¹³C NMR (CDCl₃, 50 MHz): δ 12.5, 12.8, 14.1, 26.3, 30.4, 37.2, 41.8, 42.3, 72.4, 122.9, 123.4, 124.5, 128.0, 129.5, 130.7, 133.9, 134.4, 135.2, 135.4, 136.7, 139.8, 142.5, 148.2, 152.4, 161.3, 165.7, 169.4, 193.5.

FT-IR (cm⁻¹): 3381, 3035, 2957, 2924, 2858, 1753, 1669, 1589, 1545, 1464, 1378, 1196, 970, 709.

LRMS (ESI) m/z (relative intensity): 959.55 (M⁺+K) (9%), 943.50 (M⁺+Na) (60%), 921.60 (M⁺+H) (31%). Exact calculated mass: 920.47.

### Example 2. Synthesis of CCXXI, (all-E)-3S,3′S-astaxanthin-di-N-nicotinoyl-β-alaninate ((1S)-3,5,5-trimethyl-2-oxo-4-[(1E,3E,5E,7E,9E,11E,13E,15E,17E)-3,7,12,16-tetramethyl-18-[(4S)-2,6,6-trimethyl-3-oxo-4-({3-[(pyridin-3-yl)formamido]propanoyl}oxy)cyclohex-1-en-1-yl]octadeca-1,3,5,7,9,11,13,15,17-nonaen-1-yl]cyclohex-3-en-1-yl 3-[(pyridin-3yl)formamido]propanoate)

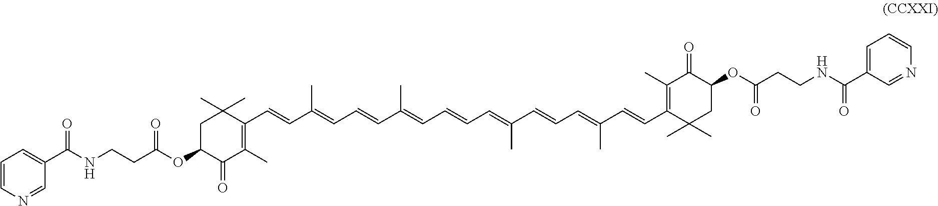 [SMILES: CC1=C(/C=C/C(C)=C/C=C/C(C)=C/C=C/C=C(C)/C=C/C=C(C)/C=C/C2=C(C)C(=O)[C@@H](OC(=O)CCNC(=O)c3cccnc3)CC2(C)C)C(C)(C)C[C@H](OC(=O)CCNC(=O)c2cccnc2)C1=O]

The precursor 3-(pyridin-3-ylformamido)propanoic acid used for the esterification of astaxanthin was prepared by reacting pyridine-3-carbonyl azide with the aminoacid (β-alanine) according to the following procedure: A 100 mL one-necked round bottom flask was charged with 1.707 g (13.87 mmol) of nicotinic acid and 45 mL of dry dichloromethane under nitrogen atmosphere. The solution was stirred and cooled in an ice bath while 3.357 g (17.51 mmol) of 1-ethyl-3-(3-dimethylaminopropyl) carbodiimide was added. After a further 15 min in the ice bath, 2.627 g (40.41 mmol) of sodium azide was added. The ice bath was removed, and the reaction mixture was stirred for 20 h at room temperature. The reaction was monitored by silica gel TLC using as mobile phase (70:20/5/5, v/v/v/v) benzene/ethanol/acetone/acetic acid. The reaction solution was washed with brine (1×30 mL) and the bottom organic layer was removed. The remaining aqueous layer was extracted with dichloromethane (2 mL). The dichloromethane fractions were combined and washed with water (4×30 mL), the bottom organic layer was dried over anhydrous magnesium sulfate and filtered. The solvent was removed under vacuum by rotary evaporation at 30° C. 2546 mg of crude was obtained and the content of pyridine-3-carbonyl azide in the crude was \tilde{ }80% w/w NMR (CDCl₃, 200 MHz): δ 7.44 (dd, 1H, J=4.7, 7.9 Hz), 8.30 (ddd, 1H, J=1.9, 2.0, 8.0 Hz), 8.84 (dd, 1H, J=1.6, 4.8 Hz), 9.17 (d, 1H, J=1.9 Hz). A 50 mL one-necked round bottom flask was charged with 5 mL of a solution of β-alanine (1.213 g, 13.5 mmol) in carbonate buffer 0.5 M pH 10 under nitrogen atmosphere and at room temperature. 2.0 g (13.5 mmol) of pyridine-3-carbonyl azide (2.4 g of the crude) was dissolved in 1 mL of dichloromethane, and the solution was added in small aliquots every two minutes to the aminoacid solution. The pH was measured with a test strip after each addition and adjusted with carbonate buffer 0.5M, if needed, to pH 8. The reaction was monitored by silica gel TLC with the mobile phase (70/20/5/5, v/v/v/v) benzene/ethanol/acetone/acetic acid. After 40 minutes, 1.9 mL of a solution 10 M of hydrochloric acid was added until reaching pH 4 and it was cooled to 4° C. (rate of cooling: 1° C. per minute). The product crystallized and the solid was isolated by filtration. The crystals were washed with a hydrochloric acid aqueous solution pH 4 (3×5 mL) and dried. 1.127 g (5.8 mmol) of 3-(pyridin-3-ylformamido)propanoic acid were obtained, 43% yield. ¹H NMR (D₂O, 200 MHz): δ 2.60 (t, 2H), 3.58 (t, 2H), 7.61 (m, 1H), 8.22 (m, 1H), 8.64 (m, 1H), 8.80 (s, 1H).

Crude free astaxanthin was obtained by de-esterification of a purified algae extract (Haematococcus pluvialis) containing \tilde{ }10 w/w total astaxanthin as free, mono-esterified and di-esterified. The content of free astaxanthin in the final de-esterification crude is 55% w/w.

198.9 mg (0.33 mmol) of free ((all-E))-3S,3′S-astaxanthin (365.6 mg of final de-esterification crude), 458 mg (2.35 mmol)₃-(pyridin-3-ylformamido)propanoic acid and 8 mL of dichloromethane were added to a 50 mL one-necked round bottom flask under nitrogen atmosphere. The solution is stirred and cooled in an ice bath. After 15 minutes, 1253 mg (6.53 mmol) of 1-ethyl-3-(3-dimethylaminopropyl) carbodiimide and 809 mg (6.6 mmol) of 4-dimethylaminopyridine were added. The reaction was allowed to reach room temperature and the mixture was stirred for 24 hours until the free astaxanthin was consumed. The reaction was monitored by silica gel TLC with the mobile phase hexane/ethyl acetate/dichloromethane/acetic acid (29/48/20/3, v/v/v/v). The reaction solution was extracted with 0.25M citric acid solution (4×15 mL) and water (3×30 mL). The dichloromethane layer was dried over anhydrous magnesium sulfate. The solvent was removed under reduced pressure by rotary evaporation. The residue was washed with distilled hexane (2×2 mL), and then dissolved in a 30/9 v/v mixture of methanol/hexane. Hexane was added to reach an hexane/methanol ratio 60/40 v/v. The methanol layer was extracted with hexane (4×3 mL). The methanol phase was concentrated by rotary evaporation under reduced pressure and it was purified by preparative HPLC. The chromatographic conditions were mobile phase acetonitrile/water 95/5, flow 15 mL/min, column C₁₈ Shimpack 250×20 mm, 10 mm. 131.4 mg (0.138 mmol) of compound CCXXI was obtained, 42.0% yield.

¹H NMR (CDCl₃, 200 MHz): δ 1.25 (s, 6H), 1.38 (s, 6H), 1.93 (s, 6H), 2.00 (s, 6H), 2.01 (s, 6H), 2.09 (m, 4H), 2.73 (t, 4H, J=5.8 Hz), 3.80 (m, 2H), 3.98 (m, 2H), 5.66 (dd, 2H, J=6.9; 12.6 Hz), 6.17 (m, 2H), 6.30 (m, 2H), 6.30 (m, 2H), 6.43 (m, 2H), 6.46 (m, 2H), 6.66 (m, 2H), 6.68 (m, 2H), 7.37 (ddd, 2H, J=0.5; 4.8; 7.9 Hz), 8.08 (t, 2H, J=5.8 Hz), 8.29 (dt, 2H, J=7.9; 1.9 Hz), 8.71 (dd, 2H, J=4.8; 1.9 Hz), 9.17 (d, 2H, J=1.9 Hz).

¹³C NMR (CDCl₃, 50 MHz): δ 12.5, 12.7, 14.0, 26.2, 30.4, 35.0, 35.9, 37.2, 42.3, 53.4, 71.4, 122.7, 123.1, 124.5, 127.9, 129.9, 130.7, 133.9, 134.3, 135.1, 135.5, 136.6, 139.9, 142.7, 148.7, 151.9, 162.1, 165.5, 171.3, 194.9.

FT-IR (cm⁻¹): 3334, 3035, 2964, 2926, 2868, 1742, 1670, 1591, 1541, 1472, 1367, 1306, 1244 1180, 970, 709.

LRMS (ESI) m/z (relative intensity): 988.35 (M⁺+H+K) (48%), 972.30 (M⁺+H+Na) (31%), 950.50 (M⁺+2H) (21%). Exact calculated mass: 948.50.

### Example 3. Synthesis of CCXXV (Mixture of CCXXII, CCXXIII and CCXXIV), (all-E)-3S,3′S-astaxanthin-di-N-nicotinoyl-alaninate ((1S)-3,5,5-trimethyl-2-oxo-4-[(1E,3E,5E,7E,9E,11E,13E,15E,17E)-3,7,12,16-tetramethyl-18-[(4S)-2,6,6-trimethyl-3-oxo-4-({2-[(pyridin-3-yl)formamido]propanoyl}oxy)cyclohex-1-en-1-yl]octadeca-1,3,5,7,9,11,13,15,17-nonaen-1-yl]cyclohex-3-en-1-yl 2-[(pyridin-3-yl)formamido]propanoate)

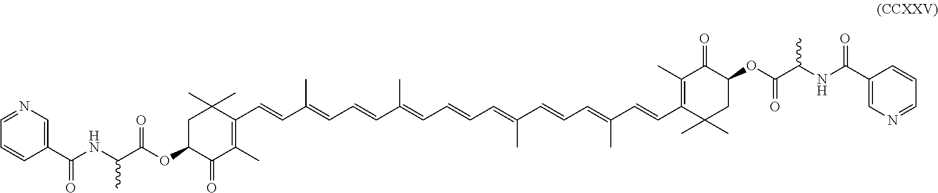

The precursor (2S)-2-(pyridin-3-ylformamido)propanoic acid used for the esterification of astaxanthin was prepared by reacting pyridine-3-carbonyl azide with L-alanine according to the following procedure: A 25 mL one-necked round bottom flask was charged with 3.5 mL of a solution of L-alanine (0.891 g, 10.0 mmol) in carbonate buffer 0.83 M under nitrogen atmosphere at room temperature. 1.5 g (10.1 mmol) of pyridine-3-carbonyl azide (2.12 g of the crude containing \tilde{ }80% w/w of the azide) was dissolved in 2 mL of dichloromethane, and it was added in small aliquots every two minutes to the L-alanine solution. The pH was measured with strip test and adjusted with carbonate buffer, if needed, to pH 8. The reaction was monitored by silica gel TLC with the mobile phase benzene/ethanol/acetone/acetic acid (70/20/5/5, v/v/v/v). After 40 minutes, the reaction solution was extracted with dichloromethane (3×4 mL). The organic layer was discarded. 1 mL of a solution 10 M of hydrochloric acid was slowly added to aqueous layer, until pH 4 was reached and it was cooled to 4° C. (rate of cooling: 1° C. per minute). The product crystallized and the solid was isolated by filtration. The crystals were washed (3×0.4 mL) with a hydrochloric acid aqueous solution pH 4 and dried. 1.050 g (5.4 mmol) of (2.5)-2-(pyridin-3-ylformamido)propanoic acid was obtained, 54% yield.

0.201 g (0.337 mmol) of free (all-E)-3S,3′S-astaxanthin (0.391 g of final de-esterification crude), 0.515 g (2.65 mmol) (2S)-2-(pyridin-3-ylformamido)propanoic and 5 mL of dichloromethane were charged in a 50 mL one-necked round bottom flask under nitrogen atmosphere. The solution is stirred and cooled in an ice bath. 1.274 g (6.65 mmol) of 1-Ethyl-3-(3-dimethylaminopropyl) carbodiimide was added. After 18 minutes, 0.898 g (7.35 mmol) of 4-dimethylaminopyridine was added. The reaction was allowed to reach room temperature and the mixture was stirred for 24 hours until the free astaxanthin was consumed. The reaction was monitored by silica gel TLC with the mobile phase hexane/ethyl acetate/dichloromethane/acetic acid (29/48/20/3, v/v/v/v). The reaction solution was extracted with 0.25M citric acid solution (4×15 mL) and water (3×20 mL). The dichloromethane layer was dried over anhydrous magnesium sulfate. The solvent is removed under reduced pressure. The residue was washed with distilled hexane (2×2 mL), and then dissolved in a 30/9 mixture of methanol/hexane v/v. Hexane and water were added to reach hexane/methanol/water with a solvent ratio composition of 60/39/1 v/v/v. The methanol layer was extracted with hexane (4×3 mL), concentrated under reduced pressure by rotary evaporation. HPLC analysis (acetonitrile/water 95/5, 1 mL/min, Agilent Eclipse XDB-C_{18 4.6} mm×12.5 mm, 5μμ) of the crude showed three peaks corresponding to the diester product with the following retention times and relative area % at 480 nm: 10.9 m, 25.8%; 12.6 m, 49.0% and 14.6 min, 25.2% (area ratio 1:2:1). The peaks correspond to the three diastereomers expected to be formed by racemization of the acylating agent. The diastereomers were separated by preparative HPLC. The chromatographic conditions were mobile phase acetonitrile/water 95:5, flow 15 mL/min, column C₁₈ Shimpack 250×20 mm, 10 mm. The total mass of the three isolated diastereomers was 0.255 g (0.269 mmol), 80% yield. The most abundant diastereomer was characterized by NMR, FTIR and MS.

¹H NMR (CDCl₃, 200 MHz): δ 1.24 (s, 6H), 1.38 (s, 6H), 1.62 (d, 3H, J=5.8 Hz), 1.69 (d, 3H, J=5.8 Hz), 1.91 (s, 6H), 1.99 (s, 6H), 2.00 (s, 6H), 2.06 (m, 4H), 2.40 (m, 2H), 4.92 (m, 2H), 5.59 (m, 2H), 6.16 (m, 2H), 6.30 (m, 4H), 6.42 (m, 2H), 6.43 (m, 2H), 6.67 (m, 2H), 6.68 (m, 2H), 7.40 (dd, 2H, J=0.8, 4.8, 7.8 Hz), 8.15 (dt, 2H, J=0.8, 4.2, 12.2 Hz), 8.70 (dd, 2H, J=1.5, 4.8 Hz), 9.05 (d, 2H, J=1.8 Hz).

¹³C NMR (CDCl₃, 50 MHz): δ 12.48, 12.72, 13.99, 18.22, 18.42, 26.21, 30.36, 30.40, 37.10, 42.24, 48.64, 48.74, 71.94, 72.25, 122.82, 122.89, 123.36, 124.51, 127.94, 128.00, 129.62, 129.77, 130.63, 133.80, 134.36 134.4, 135.09, 135.15, 135.25, 135.34, 136.62, 139.74, 142.42, 142.51, 148.17, 148.20, 152.17, 152.23, 161.13, 161.37, 164.89, 164.95, 165.07, 165.13, 172.05, 172.51, 193.43, 193.61

FT-IR (cm⁻¹): 3339, 3038, 2966, 2934, 2870, 1747, 1674, 1593, 1541, 1472, 1367, 1306, 1244, 1180, 968, 709.

LRMS (ESI) m/z (relative intensity): 972.50 (M⁺+H+Na) (68%), 950.50 (M⁺+2H) (32%). Exact calculated mass: 948.50.

### Example 4. Synthesis of CCXXIX (Mixture of CCXXVI, CCXXVII and CCXXVIII), (all-E)-3S,3′S-astaxanthin-di-N-nicotinoyl-valinate, (1S)-3,5,5-trimethyl-2-oxo-4-[(1E,3E,5E,7E,9E,11E,13E,15E,17E)-3,7,12,16-tetramethyl-18-[(4S)-2,6,6-trimethyl-4-{[(2S)-3-methyl-2-[(pyridin-3-yl)formamido]butanoyl]oxy}-3-oxocyclohex-1-en-1-yl]octadeca-1,3,5,7,9,11,13,15,17-nonaen-1-yl]cyclohex-3-en-1-yl (2S)-3-methyl-2-[(pyridin-3-yl)formamido]butanoate

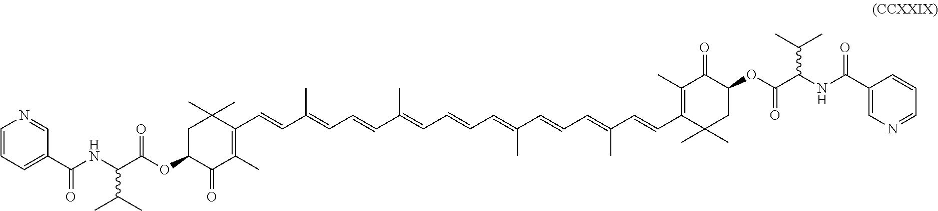 [SMILES: CC1=C(/C=C/C(C)=C/C=C/C(C)=C/C=C/C=C(C)/C=C/C=C(C)/C=C/C2=C(C)C(=O)[C@@H](OC(=O)C(NC(=O)c3cccnc3)C(C)C)CC2(C)C)C(C)(C)C[C@H](OC(=O)C(NC(=O)c2cccnc2)C(C)C)C1=O]

The precursor (2.5)-3-methyl-2-(pyridin-3-ylformamido)butanoic acid used for the esterification of astaxanthin was prepared by reacting pyridine-3-carbonyl azide with L-valine according to the following procedure: A 50 mL one-necked round bottom flask was charged with 3.5 mL of a solution of L-valine (119 mg, 0.566 mmol) in carbonate buffer 0.5 M pH 10 under nitrogen atmosphere at room temperature. 144 mg (0.97 mmol) of pyridine-3-carbonyl azide (180 mg of the crude containing \tilde{ }80% w/w of the azide) was dissolved in 2 mL of dichloromethane, and it was added to the L-valine solution, in aliquots every two minutes. The pH was measured with strip test and adjusted with carbonate buffer, if needed, to pH 8. The reaction was monitored by silica gel TLC with the mobile phase benzene/ethanol/acetone/acetic acid (70/20/5/5, v/v/v/v). After 40 minutes, 1.8 mL of a solution 10 M of hydrochloric acid was slowly added to aqueous layer, until pH 4 was reached and it was cooled to 4° C. (rate of cooling: 1° C. per minute). The product crystallized and the solid was isolated by filtration. The crystals were washed (3×5 mL) with a hydrochloric acid aqueous solution pH 4 and dried. 62 mg (0.28 mmol) of (2.5)-3-methyl-2-(pyridin-3-ylformamido)butanoic acid was obtained, 49% yield.

22.42 mg (0.037 mmol) of free (all-E)-3S,3′S-astaxanthin (43.70 mg of final de-esterification crude), 62 mg (0.279 mmol) (2.5)-3-methyl-2-(pyridin-3-ylformamido)butanoic acid and 1.5 mL of dichloromethane were charged in a 10 mL one-necked round bottom flask under nitrogen atmosphere. The solution is stirred and cooled in an ice bath. After 10 minutes, 162 mg (0.845 mmol) of 1-ethyl-3-(3-dimethylaminopropyl) carbodiimide and 96 mg (0.786 mmol) of 4-dimethylaminopyridine were added. The reaction was allowed to reach room temperature and the mixture was stirred for 24 hours until the free astaxanthin was consumed. The reaction was monitored by silica gel TLC with the mobile phase (29/48/20/3, v/v/v/v) hexane/ethyl acetate/dichloromethane/acetic acid. The reaction solution was extracted with 0.25M citric acid solution (1×10 mL) and water (3×10 mL). The dichloromethane layer was dried over anhydrous magnesium sulfate and the solvent was removed under reduced pressure. The residue was washed with distilled hexane (3×0.5 mL), and then dissolved in 1 mL of methanol. Hexane and water were added to reach hexane/methanol/water with a solvent ratio composition of 59/39/2 v/v/v. The methanol layer was extracted with hexane (2×2.5 mL), concentrated under reduced pressure by rotary evaporation.

HPLC analysis (acetonitrile/water 95/5, 1 mL/min, Agilent Eclipse XDB-C18 4.6mm×12.5 mm, 5 μm) of the crude showed three peaks corresponding to the diester product with the following retention times and relative area % at 480 nm: 12.6 m, 25.7%; 15.0 m, 49.1% and 17.6 min, 25.2% (area ratio 1:2:1). The peaks correspond to the three diastereomers expected to be formed by racemization of the acylating agent. The diastereomers were separated by preparative HPLC. The chromatographic conditions were mobile phase acetonitrile/water 95:5, flow 15 mL/min, column C₁₈ Shimpack 250×20 mm, 10 mm. The total mass of the three isolated diastereomers was 12.1 mg (0.012 mmol), 32% yield. The most abundant diastereomer was characterized by NMR, FTIR and MS.

¹H NMR (CDCl₃, 200 MHz): δ 1.09 (d, 6 H, J=6.9 Hz), 1.15 (d, 3H, J=5.8 Hz), 1.17 (d, 3H, J=5.8 Hz), 1.23 (s, 3H), 1.25 (s, 6H), 1.36 (s, 3H), 1.37 (s, 3H), 1.82 (s, 6H), 1.94 (s, 6H), 1.99 (s, 6H), 2.07 (m, 4H), 2.45 (m, 2H), 4.87 (dd, 1 H, J=4.6, 8.6 Hz), 4.95 (dd, 1 H, J=4.6, 8.6 Hz), 5.55 (m, 2H), 6.14-6.8 (m, 14H), 7.42 (ddd, 2H, J=0.5, 4.8, 8 Hz), 8.16 (dm, 2H, J=8 Hz), 8.75 (dd, 1H, J=4.8; 1.9 Hz), 8.76 (dd, 1H, J=4.8; 1.9 Hz), 9.05 (d, 1H, J=1.9 Hz), 9.07 (d, 1H, J=1.9 Hz).

¹³C NMR (CDCl₃, 50 MHz): δ 26.30, 26.35, 29.68, 30.43, 31.53, 31.57, 37.17, 37.18, 42.50, 42.56, 57.55, 57.75, 71.95, 72.24, 122.93, 122.99, 123.47, 123.49, 124.57, 128.19, 128.23, 129.97, 130.05, 130.69, 133.85, 134.44, 134.47, 135.09, 135.15, 135.03, 135.14, 135.27, 135.31, 136.69, 136.71, 139.78, 139.81, 142.44, 148.07, 148.18, 152.43, 152.48, 161.03, 161.06, 165.40, 165.58, 170.82, 171.35, 193.21, 193.40.

FT-IR (cm⁻¹): 3332, 3034, 2964, 2930, 2872, 1744, 1669, 1591, 1553, 1535, 1468, 1452, 1371, 1310, 1242, 1195, 1159, 1092, 1028, 968, 918, 829, 732, 708.

LRMS (ESI) m/z (relative intensity): 1044.5 (M⁺+H+K) (29%), 1028.55 (M⁺+H+Na) (41%), 1006.55 (M⁺+2H) (32%). Exact calculated mass: 1004.57

### Example 5. Conversion of Prodrug to Parent Compound

Formation of astaxanthin in resuspended Caco-2 cells previously frozen.

Human carboxylesterase 1 (hCE-1) and carboxylesterase 2 (hCE-2) are serine esterases involved in both drug metabolism and activation of prodrugs. The expression pattern of human carboxylesterase in Caco-2 (human colon adenocarcinoma) cell line is similar to human liver that expresses a much higher level of hCE-1 and lower level of hCE-2 (Drug Metab. Pharmacokinet. 21 (3): 173-185 (2006)). Caco-2 cell homogenates was therefore used as an in vitro model of liver metabolism to demonstrate conversion of the prodrugs of the present invention into the parent compound.

Therefore, the formation of astaxanthin from compounds CCXX, CCXXI, CCXXII and CCXXVI was tested with Caco-2 cells that were frozen at −80° C. and thawed before testing. The freezing/thawing process without cryopreservants compromised the integrity of the membrane, as confirmed by positive staining of the dead cells with a dye exclusion method using trypan blue. Dead cells are not selective to compounds passing through the compromised membrane, eliminating the transport and permeation barriers to access the cell content and metabolic enzymes including the carboxylesterases.

The Caco-2 (colorectal adenocarcinoma) cellular line was obtained from Cordoba Cell Bank (CCB, CIQUIBIC, Cordoba, Argentina), #ATCC Passage: 18. #CCB Passage: 4. Culture conditions: Low Glucose DMEM (1 g/L)+Penicillin-Streptomycin+20% Fetal Bovine Serum (SFB). Cells were cultured in a 37° C. incubator with 5% CO₂ on cell culture dishes. Once they reached confluence, cells were washed twice with Phosphate-buffered saline (PBS) and lifted after trypsinization. For trypsinization, 1 mL of trypsin was added, incubated for a few minutes in the 37° C. incubator, neutralized with supplemented culture medium and cells were collected in 15 mL tubes (Falcon). The tubes were centrifuged for 5 min at 1000 rpm, the supernatant was removed, and the cells were resuspended in supplemented medium. To determine the amount of viable (living) cells, a ½ dilution with trypan blue dye was made and counted in an automatic cell counter (more than 90% of live cells were measured). Finally, the tubes were centrifuged again, and cells were resuspended at a concentration of 5 million/mL in non-supplemented DMEM and placed in a −80° C. freezer. To determine the amount of non-viable cells after freezing and thawing, a ½ dilution with trypan blue dye was made and they were counted in an automatic cell counter and the dead cells were above 95%. Pools of unviable (“leaking”) cell homogenates were subsequently used for incubations with the compounds of interest.

A 1 mL frozen aliquot containing 5 million/mL of unviable Caco-2 cells was thawed and warmed at 37° C. A 0.010 mL aliquot of a stock solution of the compound in DMSO (2.5 mM) was added to the cells and incubated at 37 ° C. At selected timepoints, a 0.2 mL aliquot was removed and mixed with 0.5 mL of methanol containing retinyl acetate (0.011 mM) as an internal standard. The methanolic mixture was vigorously vortexed and 1 mL of chloroform was added to extract unreacted compound, astaxanthin and related compounds as well as the internal standard. After vigorous vortexing, the mixture was centrifuged at 8000 rpm for 10 min at 4° C. The chloroform phase was removed and dried under nitrogen flow. 1 mL of the sample solvent (THF/acetonitrile with 1% Formic acid) was added to the residue, mixed vigorously and filtered through a PVDF 0.22 um filter in a HPLC vial. The filtrate was stored in the autosampler at 5° C. and injected in the HPLC to measure the amount of internal standard, unreacted compound, free astaxanthin and its related compounds. The HPLC equipment was a UHPLC Nexera XR (Shimadzu)/PDA SPD-M3OA (Shimadzu) with a Shimpack GIST C18 100×2.1 mm, 2 um column at 40° C. Two HPLC methods were used, gradient and isocratic depending on the prodrug. Gradient method: flow 0.2 mL/m, mobile phase A (MPA): water with formic acid 0.1%, mobile phase B (MPB): acetonitrile with formic acid 0.1%, gradient: time 0 m 60/40, 2.5 m 10/90, 10.4 m 10/90. UV(PDA) detector 380 and 325 nm (for retinyl acetate). Isocratic method: same conditions but the solvent composition was constant, MPA/MPB 20/80.

Conversion to trans-astaxanthin was observed for CCXX, CCXXI and CCXXII. Compound CCXX showed an expectedly higher conversion efficiency to trans-astaxanthin than CCXXI and CCXXII. Compound CCXXVI was stable over the same time frame with only a small amount of conversion to astaxanthin monoester.

### Experimental Models

NAFLD/NASH: The murine STAM model is a model that recapitulates the same disease progression as human NASH/HCC (See, e.g., WO2011/013247; Takakura et al. Anticancer Res, 2014; 34(9):4849-55). In this model, male C57BL/6 mice aged two days are given a single dose of streptozotocin to reduce insulin secretory capacity. At four weeks of age the mice start a high-fat diet. This model has a background of late type 2 diabetes which progresses into fatty liver, NASH, fibrosis and consequently liver cancer (HCC). The disease progresses in a relatively short period of time, and liver cancer is developed in 100% of animals at 20 weeks of age. The STAM™ model is able to reproduce many of the pathological features of human NASH including:
1. Ballooning degeneration of cells, a characteristic pathological feature of human NASH.
2. Burned-out NASH, in which lipid droplets decrease as fibrosis progresses.
3. Progression of fibrosis occurring around the central vein.
4. A mild rise in ALT (a liver injury marker).
5. Increase in NASH markers such as CK-18.
6. Increase in human HCC markers such as: glutamine synthase, glypican-3 and AFP.

NASH was induced in male C57BL76 mice by a single subcutaneous injection of 200 pg of streptozotocin (STZ, Sigma, MO, USA) at 2 days after birth and continuous feeding after 4 weeks of age (day 28±2) with a high-fat diet given ad libitum.

Following induction of NASH, the mice are randomized into individual study groups of 8 mice each at 6 weeks of age (day 42±2) based on body weight, the day before the start of treatment. One day following randomization, the mice are administered a once-daily oral treatment from 6 weeks of age plus one day (day 43±2, treatment Day 1) to 9 weeks of age (hereinafter known as the “treatment period”). One study group of mice receives vehicle only and serve as a vehicle control group, a second study group serves as a positive control and receives telmisartan, and the remaining study groups receive different xanthophyll derivatives or different doses of xanthophyll derivatives of the present invention.

Individual body weights are measured daily during the treatment period. Survival, clinical signs and behavior of mice are also monitored daily. All mice are sacrificed at 9 weeks of age. Blood samples are collected from all mice and the liver from each mouse is removed for analysis. The plasma from each blood sample is analyzed for alanine aminotransferase (ALT) as an indicator of liver function and disease progression. The livers from all sacrificed mice are weighed in grams. The removed livers are fixed in formalin and embedded in paraffin, and cross sections are then prepared. Liver cross-sections are subjected to hematoxylin and eosin (H&E) staining using standard techniques for histological assessment of hepatic steatosis, lobular inflammation and hepatocellular ballooning. The level of steatohepatitis severity in each liver cross-section is indicated by NAFLD Activity Scores of 0-5 in 3 randomly selected fields of H&E-stained liver cross-sections at ×50 magnification for evaluation of steatosis and ×200 magnification each for evaluation of inflammation and evaluation of ballooning. The NAFLD Activity Score is the unweighted sum of the following: 1) hepatic steatosis score (0-3); 2) lobular inflammation score (0-2); 3) hepatocellular ballooning score (0-2). Comparison between the study group and positive control group is performed using a two-tailed, heteroscedastic (two-sample unequal variance) Student's T-Test. A P-value of <0.05 is considered statistically significant following a Bonferroni post-hoc statistical correction analysis for multiple groups (Bonferroni Multiple Comparison Test).

As another animal model of non-alcoholic steatohepatitis, a male C57Bl/6J mouse are used. Mice are fed a cholesterol-containing cocoa butter diet ad libitum to induce non-alcoholic steatohepatitis.

For an alcoholic steatohepatitis model, alcohol is added to a Lieber deCarli (LdC) diet, in which normal mouse chow is replaced by a high-fat, nutritionally complete liquid diet ad libitum. Mice are typically given 3 days of the LdC diet for accustomization purposes, and then alcohol is added at increasing concentrations of 2.1%, 4.2%, and 6.4% v/v for 3-day blocks, respectively.

Mouse models of diet-induced obesity (DIO) and nonalcoholic steatohepatitis (NASH): Xanthophyll derivative of the present invention can inhibit inflammation and fibrosis in the liver and adipose tissue. Obesity-associated metabolic abnormalities, inflammation and fibrosis can be developed in diet-induced obesity (DIO) and nonalcoholic steatohepatitis (NASH) mouse models ((J Nutr Biochem. 2017; 43: 27-35, B. Kim et al.). Obese individuals have a high risk of developing nonalcoholic fatty liver disease (NAFLD). When adipocytes become hypertrophied, macrophages infiltrate into the adipose tissue and produce pro inflammatory cytokines such as tumor necrosis factor α (TNFα) and interleukin (IL)-6. The cytokines stimulate the release of free fatty acids from adipocytes, resulting in high influx of free fatty acids into the liver, leading to the development of liver steatosis. Steatosis can progress to nonalcoholic steatohepatitis (NASH), which is characterized by inflammation and fibrosis. Liver fibrosis is a scarring process that can progress to cirrhosis, the primary cause for hepatocellular carcinoma. Tissue macrophages can be classified as classically activated, proinflammatory M1 and alternatively activated, anti-inflammatory M2 type. CD11c is a well-known marker of M1 macrophages, which produce proinflammatory cytokines, such as TNFα, IL-6 and monocyte chemoattractant protein-1 (MCP-1). M2 macrophages are the major resident macrophages in lean adipose tissue and are characterized by high expression of CD206, arginase-1 (Arg-1), macrophage galactose-type calcium-type lectin 1/CD301a and IL-10. Xanthophyll derivative of the present invention can prevent obesity-associated inflammation and fibrosis as demonstrated in mouse models of DIO and NASH. For example, xanthophyll derivative of the present invention can decrease the expression of fibrogenic genes such as LUM, COL6A1 and COL6A3. Also, the xanthophyll derivative of the present invention can decrease the expression of fibrogenic genes, including TGFβ1, LUM, TNC, COL1A1, COL6A1 and COL6A3.

Obesity-associated metabolic abnormalities, inflammation and fibrosis can be developed in the diet-induced obesity (DIO) and nonalcoholic steatohepatitis (NASH) mouse models as disclosed in J Nutr Biochem. 2017, 43: 27-35, B. Kim et al. Briefly, male C57BL/6J mice are fed a low-fat (6% fat, w/w), a high-fat/high-sucrose control (HF/HS; 35% fat, 35% sucrose, w/w), or a HF/HS containing a xanthophyll derivative of the present invention (XHF/HS) for 30 weeks. To induce NASH, another set of mice is fed a HF/HS diet containing 2% cholesterol (HF/HS/HC) a HF/HS/HC with xanthophyll derivative of the present invention (XHF/HS/HC) for 18 weeks. Body weights and food consumption are recorded weekly, and blood samples are collected monthly from the lateral tail vein. At the end of the feeding period, mice are starved and anesthetized. Blood is collected in a tube containing EDTA and centrifuged for plasma collection. Liver, epididymal adipose tissue (eAT), retroperitoneal adipose tissue (rAT), gastrocnemius muscle and spleen are harvested and snap frozen in liquid nitrogen or fixed in 10% formalin. Plasma and tissue samples are stored at −80° C. until use. Mice fed the HF/HS diet or the HF/HS/HC diet are referred to as DIO or NASH mice. Plasma total cholesterol, triglyceride and glucose are compared. Macrophage infiltration and fibrosis in eAT of DIO mice are assessed by measuring the expression of macrophage markers (F4/80, CD68, CD11c, MCP-1, CD206, Arg-1, IL-10, Caspase (Carp)-3 and Casp-9 mRNA) by qRT-PCR analysis and phenotypes in eAT. The expression of genes involved in fibrosis (collagen type I, α1 (COL1A1), collagen type VI, α1 (COL6A1) and COL6A3) is measured in eAT. The expression of lysyl oxidase-like 2 (LOXL2), an enzyme associated with cross-linking elastin and collagen for fibrosis, and hypoxia-inducible factor 1-α (HIF1α) are measured and compared. Hepatic mRNA levels of markers of macrophages (F4/80 and CD68 CD11c and MCP-1 and CD206) and fibrosis (VIM), COL1A1, COL6A1, COL6A3, matrix metalloproteinase-2 (MMP2) and HIF1α) in both models are measured and compared. Histological analysis is used to determine the presence of micro-vesicular lipid droplets in the liver. Collagen accumulation in the liver is examined using Gomori's trichrome staining.

Human in vitro NASH model based on 3D microtissue technology: The Insphero 3D-INSIGHT™ human in vitro NASH model is engineered to incorporate the primary human hepatocytes, hepatic stellate cells, Kupffer cells (KCs) and liver endothelial cells (LECs) (See, e.g., US Patent Application US2019/0316093 Messner et al.; Poster P06-032, Eurotox 2019, Simon Strobel et al.). This model includes all the liver cell types that play a crucial role in disease initiation and progression. Upon treatment with free fatty acids and lipopolysaccharides (LPS) in diabetic medium, these microtissues show key physiological aspects of NASH. Lipotoxic NASH stimuli increased lipid accumulation in hepatocytes as microtissue secretion of pro-inflammatory markers, such as TNF-α, IL-6, IL-8, MCP-1, MIP-1α, and IP-10. Furthermore, lipotoxic stress stimuli increased expression of pro-fibrotic markers, such as collagen type I and III, and release of pro-collagen type I. This human 3D NASH model recapitulates key biological aspects of full spectrum of NAFLD diseases, including steatosis, inflammation, and fibrosis. Compatible with high-throughput screening approaches, this model is a powerful tool for assessing efficacy of NASH drugs.

A protocol for 10 days treatment with free fatty acids (FFA) and LPS in medium containing high levels of sugars is developed to recapitulate NASH pathogenesis in vitro, and analyzed characteristic markers of NASH (lipid loading, activation of proinflammatory markers, and initiation of fibrosis. To demonstrate lipid-loading within the tissues, the microtissues are fixed with 4% PAF and stained with Nile Red. Confocal microscopy is performed to visualize lipid-stained microtissues. To measure the triglyceride levels within the microtissues, the Glycerol-Triglyceride-GloTM kit (Promega, not yet commercially available) is used. Release of pro-inflammatory cytokines/chemokines is measured with the Human Magnetic Luminex Assay (R&D systems). The following control and treatments are applied to microtissues together with NASH stimuli, as indicated, to investigate their effect on NAFLD and NASH disease progression:
A) Control: medium with normal glucose level (no NASH induction)
B) Vehicle: vehicle treatment with NASH induction (NASH induction medium+FFA+LPS pulse)
C) Positive control: Elafibranor treatment with NASH induction
D) Test: Different xanthophyll derivatives or different doses of xanthophyll derivatives of the present invention treatment with NASH induction.

Luminex analysis of secreted cytokines and chemokines (IL-6, IL-8, TNF-α, IP-10, MIP-1α and MCP-1) are expected to show that NASH stimuli increase the secretion of the pro-inflammatory markers in the NASH treated samples as compared to the control (no NASH induction) after day 5 of treatment. Furthermore, treatment with xanthophyll derivatives of the present invention should decrease inflammatory markers secretion in NASH induced microtissues. Progression of fibrosis by anti-fibrotic drug treatment is reduced as demonstrated by immunohistochemical staining of collagen I after 7 days of treatment. In another approach, with xanthophyll derivatives of the present invention is expected to show a dose dependent down regulation of the pro-fibrotic genes alpha-SMA (Acta2), Collagen 3A1 and Collagen 4A1.

### Mitochondrial Respiration during the Activation of Hepatic Stellate Cells

Upon liver injury, quiescent hepatic stellate cells (qHSCs) transdifferentiate to myofibroblast-like activated HSCs (aHSCs), which are primarily responsible for the accumulation of extracellular matrix proteins during the development of liver fibrosis. Therefore, aHSCs may exhibit different energy metabolism from qHSCs to meet their high energy demand (J Nutr Biochem. 2019, 71: 82-89, M. Bae et al.). To characterize the energy metabolism of qHSCs and aHSCs, mouse primary HSCs are cultured on uncoated plastic dishes for 7 days for spontaneous activation in the presence or absence of different xanthophyll derivatives or different doses of xanthophyll derivatives of the present invention. qHSCs (one day after isolation) and aHSCs treated with or without different xanthophyll derivatives or different doses of xanthophyll derivatives of the present invention for 7 days are used to determine parameters related to mitochondrial respiration using a Seahorse XFe24 Extracellular Flux analyzer. aHSCs has significantly higher basal respiration, maximal respiration, ATP production, spare respiratory capacity, and proton leak than those of qHSCs. However, xanthophyll derivatives of the present invention should prevent most of the changes occurring during HSC activation and can improve mitochondrial cristae structure with decreased cristae junction width, lumen width, and the area in primary mouse aHSCs. Furthermore, qHSCs isolated from mice fed with xanthophyll derivatives of the present invention should have lower mitochondrial respiration and glycolysis than control qHSCs.

Cellular ROS Accumulation HSCs: Cellular ROS levels can be measured in LX-2 cells as previously described in US Patent Application US2016/0287534A1; Lee et al. J. Nutr. Biochem. 2013, vol. 25, iss. 4, pp. 404-411. Briefly, LX-2 cells are plated in a black 24-well plate (Wallac Oy, Turku, Finland). When cells reach \tilde{ }90% confluency, they are pre-incubated with 5, 10 or 25 μM xanthophyll derivative for 24 h and subsequently stimulated with 2 ng/mL TGFβ1 or 10 μM tert-butyl hydrogen peroxide (tBHP, Sigma, St. Louis, Mo.) for additional 24 h. Cells are then incubated with 5 μM dichlorofluorescein (Sigma, St. Louis, Mo.) for 30 min and fluorescence is read at an excitation wavelength of 485 nm and an emission wavelength of 530 nm. The data are expressed as fluorescent intensity per μg of cell protein.

Expression of HDAC₉ and Myocyte Enhancer Factor2(MEF2) in HSCs (see e.g., US Patent Application US2016/0287534A1, Ji-Young Lee): Briefly, HDACs are a class of enzymes that remove acetyl groups from lysine residues in histones. HDACs, particularly class II HDACs, i.e., HDAC4, 5, 6, 7, 9, and 10, have been suggested to play a critical role in the activation of HSCs. Alterations in mRNA expression of the classical HDACs are examined during HSC activation. The expression of histone acetyltransferases (HATS), including p300 and general control non-repressible 5, which acetylate histones is measured. The expression of MEF2, a transcription factor, known to induce HDAC₉ expression is also measured.

Pulmonary Fibrosis: Six to eight-week-old female C57B1/6J (WT) mice are used. Animals are anesthetized intraperitoneally before tracheotomy. A single injection containing 3.5 U/kg of bleomycin (BLM) diluted in saline, or saline only (control group), is instilled intratracheally. Fourteen days after bleomycin, animals are euthanized to perform bronchoalveolar lavage and collect lung samples for biochemical and histologic analysis. The total content of lung collagen is measured by the Masson's trichome stain following the manufacturer's instructions. For the treatment group, the mice are orally administered with a xanthophyll composition of the invention.

### Primary Biliary Cirrhosis and Primary Sclerosing Cholangitis

CCl4 Fibrosis Model: Fibrosis can be induced in BALB/c male mice by bi-weekly administration of CCl₄ administered by intraperitoneal injection. CCl₄ is formulated 1:1 in oil and is injected IP at 1 mL/kg. After 2-4 weeks of fibrosis induction the xanthophyll derivatives can be administered daily by oral gavage for 2-6 weeks of treatment while continuing CCl₄ administration. At study termination livers can be formalin fixed and stained with Sirius Red stain for histopathological evaluation of fibrosis. Total collagen content can be measured by colorimetric determination of hydroxyproline residues by acid hydrolysis of collagen. Serum alanine aminotransferase (ALT) and aspartate aminotransferase (AST) can be measured by a clinical chemistry analyzer.

Intrahepatic Cholestasis Model: Experimental intrahepatic cholestasis induced by 17a-ethynylestradiol (EE2) treatment in rodents is a widely used in vivo model to examine the mechanisms involved in estrogen-induced cholestasis. Intrahepatic cholestasis can be induced in adult male mice by subcutaneous injection of 10 mg/kg 17a-ethynylestradiol (E2) daily for 5 days. Testing of xanthophyll derivatives can be performed by administration of compounds during E2 induction of cholestasis. Cholestatic effects can be quantitated by assessing liver/body weight ratio and measuring serum total bile acids and alkaline phosphatase levels can be measured using reagents and controls from Diagnostic Chemicals Ltd. and the Cobas Mira plus CC analyzer (Roche Diagnostics). For histology and mitosis measurements, liver samples from each mouse can be fixed in 10% neutral buffered formalin. Slides are stained with hematoxylin and eosin using standard protocols and examined microscopically for structural changes. Hepatocyte proliferation is evaluated by immunohistochemical staining for Ki67.

Rat ANIT Model: A compound described herein is evaluated in a chronic treatment model of cholestasis over a range of doses. This model is used to evaluate the suitability of the use of xanthophyll derivatives described herein for the treatment of cholestatic liver disorders such as bile acid malabsorption, bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, and parenteral nutrition-associated liver disease.

Rats are treated with alpha-naphthylisothiocyanate (ANIT) (0.1% w/w) in food for 3 days prior to treatment with a xanthophyll derivative described herein, at a range of doses. A noncholestatic control group is fed standard chow diet without ANIT and serves as the noncholestatic control animals (“Control”). After 14 days of oral dosing, rat serum is analyzed for levels of analytes. LLQ, lower limit of quantitation. Mean±SEM; n=5.

Levels of hepatobiliary injury indicators are measured in rat serum, such as elevated levels of circulating aspartate aminotransferase (AST), alanine aminotransferase (ALT), bilirubin and bile acids. ANIT exposure induces profound cholestasis and hepatocellular damage. A xanthophyll derivative that improves many of these indicators is useful in the treatment of the aforementioned diseases or conditions.

Reductions in the accumulation of bile acids in the liver, enhancements in bile acid excretion in the biliary tract and inhibition of bile acid synthesis is consistent with the pharmacological action of a xanthophyll derivative. An improvement in the serum conjugated bilirubin (a direct indicator for hepatic function) implies recovery from cholestasis with improved bile excretion.

### Inflammatory Bowel Disease (IBD)

Mouse Chronic DSS Colitis Model: The chronic Dextran Sodium Sulfate (DSS)-induced mouse can be used to test the therapeutic potential of xanthophyll derivatives against inflammatory bowel disease (IBD). Chronic colitis can be induced by feeding mice DSS in drinking water. For example, 2% DSS in drinking water for 5 days and regular drinking water for 5 days, then this feeding cycle can be repeated two more times with higher concentrations of DSS, 2.5% and 3%, respectively for a total of three cycles. Colitis develops approximately after the first cycle of DSS feeding, which can be monitored by loss of body weight, stool consistency and rectal bleeding. A xanthophyll derivative can be tested by administering to mice at the same time of starting 2% DSS water feeding. Alternatively, testing of a xanthophyll derivative can be performed post the first feeding cycle of 2% DSS water and regular water. During the period of administering the xanthophyll derivative to mice, the therapeutic effects can be monitored by observations on body weights, stool consistency and rectal bleeding. After euthanasia, the disease development and effects of the xanthophyll derivative can be further quantified by measuring colon weight and length, colon histology by H&E staining for inflammation and structural changes in mucosa, and protein and RNA expression of genes related to the disease.

Adoptive T-Cell Transfer Colitis Mouse Model: The adoptive T-cell transfer colitis model is accepted as a relevant mouse model for human inflammatory bowel disease (IBD). To induce colitis in this model, the CD4 T-lymphocyte population is isolated from the spleens of donor mice, subsequently a subpopulation of CD4+CD45RB high T-cells is purified by cell sorting using flow cytometry. The purified CD4+CD45RB high T-cells are injected into the peritoneal cavity of the recipient SCID mice. Colitis develops approximately three to six weeks after T-cell transfer, which can be monitored by loss of body weight (although loss of body weight can be variable), inconsistent stool or bloody diarrhea. Testing of a xanthophyll derivative can be initiated at the same time of injecting purified CD4+CD45RB high T-cells to the recipient SCID mice. Alternatively, the xanthophyll derivative can be administered two or three weeks post T-cell transfer, when colitis has already developed in the model. During the period of administering the xanthophyll derivative to mice, the therapeutic effects can be monitored by observations on body weights, stool consistency and rectal bleeding. After euthanasia, the disease development and effects of the xanthophyll derivative can be further quantified by measuring colon weight and length, colon and ileum histology by H&E staining for inflammation and structural changes in mucosa, and protein and RNA expression of genes related to the disease.

Mdr1a−/− Mouse Model: The Mdr1a−/− mouse model is a spontaneous colitis model that has been used in testing new therapies for human IBD. Loss of the Mdr1a gene in this model leads to impaired intestinal barrier function, which results in increased infiltration of gut bacteria and subsequent colitis. Under proper housing conditions, Mdr1a−/− mice can develop colitis at about 8 to 13 weeks of age. During disease progression, a disease activity index (DAI) summing the clinical observation scores on rectal prolapse, stool consistency and rectal bleeding can be used to monitor the disease. Testing of a xanthophyll derivative can be started at the initial stage of disease, generally with DAI score less than 1.0. Alternatively, administration of a xanthophyll derivative can be initiated when colitis has developed, typically with a DAI score above 2.0. Therapeutic effects of the xanthophyll derivative can be monitored by measuring the DAI, and testing can be terminated when desired disease severity has been achieved, generally with a DAI score around 5.0. After euthanasia, the disease development and effects of the xanthophyll derivative can be further quantified by measuring colon weight and length, colon histology by H&E staining for inflammation and structural changes in mucosa, and protein and RNA expression of genes related to the disease.

### Antioxidative Activity of Xanthophyll Derivatives to DPPH Radicals Scavenging and Lipid Peroxidation

DPPH radical scavenging activity: The experiment is performed following the previously described method in Yamaguchi, T., et al. Biosci. Biotechnol. Biochem. 1998; 62:1201-1204. Briefly, an aliquot of xanthophyll derivative (final concentration of 10 μM) in THF is added to DPPH (final concentration of 1 mM) in ethanol/100 mM Tri-HCl buffer (pH 7.5) (3:1, v/v). The mixture is allowed to stand for 40 min at room temperature in the dark. The absorbance at 517 nm by DPPH is measured by HPLC using a TSKgel-Octyl-80Ts column (4.6×150 mm, Tosoh, Tokyo, Japan) with a mobile phase of methanol/water (70:30, v/v) at a flow rate of 1 ml/min. The same amount of THF, without xanthophyll derivative, is added as a blank.

Microsomal lipid peroxidation: Fresh microsomes are prepared by the previously described method by Palozza, S. et al. Radic. Biol. Med. 1992; 13:127-136. Briefly, fresh microsomes are obtained from Wister rats (6 weeks, 180-200 g) by tissue homogenization with 5 volumes of ice-cold phosphate buffer (pH 7.4) containing 5% EDTA and 5% BHT. Microsomal vesicles are isolated by removal of the nuclear fraction at 19,000 rpm for 10 min, and the mitochondrial fraction is removed at 28,000 rpm for 10 min. The microsomal fraction is sedimented at 61,000 rpm for 60 min, washed once in 0.15 M KCl, and collected again at 61,000 rpm for 30 min. The membranes are homogenized again into 0.1M Tris-HCl buffer (pH 7.4), and stored at !80° C. Microsomal proteins are determined by a BCA assay kit (Pierce). Xanthophyll derivatives (final concentration of 10 nmol/mg protein) in THF are added to the microsomes, and lipid peroxidation is initiated by the addition of 50 mM AAPH. The same amount of THF, without xanthophyll derivatives, is added as the control. Reaction mixtures are shaken in air at 37° C. Lipid peroxidation products are determined as malondialdehyde (MDA) formation by measurement at 532 nm.

Rabbit erythrocyte membrane ghost system: Rabbit erythrocyte membrane ghosts are prepared by following the previously reported method of Osawa, T. et al. Biosci. Biotechnol. Biochem. 1995; 59:1609-1612. Commercially available rabbit blood (100 ml) is diluted with 150 ml of isotonic buffer solution (10 mM phosphate buffer/152 mM NaCl). After centrifugation (3500 rpm, 10 min), the blood is washed three times with 10 ml of isotonic buffer solution and lysed in 10 mM phosphate buffer, pH 7.4. Erythrocyte membrane ghosts are pelleted by centrifugation (11,000 rpm, 40 min), and the precipitate is diluted to give a suspension. Xanthophyll derivatives (final concentration of 10 nmol/mg protein) in THF are added to the erythrocyte membrane ghosts, and peroxidation is induced by tert-butyl hydroperoxide. The same amount of THF, without xanthophyll derivatives, is added to the control. After incubation at 37° C. for 20 min, 1 ml of 2.0M TCA/1.7M HCl and 2 ml of 0.67% TBA solution are added to stop the reaction. The quantity of TBA-reacting substance (TBARS) is determined at 532 nm.

Fatty acid peroxidation reaction: An appropriate amount of xanthophyll derivatives (10 uM) is added to 1 mM DHA or methyl linoleate solution. Peroxidation is initiated by adding AMVN (1 mM). Reaction solutions in THF are incubated under air in the dark at 37° C. with continuous shaking. At regular intervals, aliquots of the sample (50 μL) are withdrawn and stored at −80° C. immediately. Lipid hydroperoxide levels are determined by HPLC monitored at 234 nm. As a preliminary experiment, a major DHA peroxidation product fraction is isolated by HPLC monitored at 234 nm and identified by measuring the hydroperoxide activity of each isolated fraction using a Lipid Hydroperoxide assay kit (Cayman).

DHA hydroperoxides preparation: DHA hydroperoxides (DHA-OOH) are prepared by the reaction of soybean lipoxygenase with DHA. A 83.6-mg sample of DHA (purity 70%, NOF Co., Japan) and 8 mg lipoxygenase (100 mg, Sigma type I-B) are added to 220 mL of 200 mM borate buffer (pH 9.0). The reaction is carried out for 15 min by stirring in a dish filled with O₂ at room temperature. The reaction is terminated by HCl addition to pH<4.0, and the formed peroxides are extracted twice with an equal amount of chloroform/methanol (1:1). The collected chloroform layer is evaporated. The obtained peroxides are quantified by lipid hydroperoxide kit (Cayman) compared to a standard curve prepared by authentic 13-HPODE.

Linoleic acid hydroperoxides preparation. Methyl linoleate hydroperoxide (MLOOH) is prepared by the reaction of soybean lipoxygenase with methyl linoleate (ML). A 200-mg sample of ML and sodium deoxycholate (1.62 g) is dissolved in 240 mL of 200 mM borate buffer (pH 9.0). Lipoxygenase (100 mg, Sigma type I-B) is added to the solution and incubated for 3 h at room temperature. The formed peroxide is extracted twice with an equal amount of chloroform/methanol (1:1). The collected chloroform layer is evaporated. The obtained peroxide is purified by thin layer chromatography (TLC) and developed with n-hexane/ether (6:4). The peroxide is extracted with CHCl₃ and then the solvents are evaporated. The amount of MLOOH is calculated from the molar coefficient, ε(234nm)=25000M⁻¹ cm⁻¹, using the value of linoleic acid hydroperoxide.

### Effects of Xanthophyll Derivatives on Oxidative Stress and ROS Generation

Cell culture and fluorescence assay of the ROS generated in SH-SY5Y cells: SH-SY5Y cells, a human dopaminergic neuroblastoma cell line, are cultured in Dulbecco's modified Eagle's medium (DMEM) containing 5% fetal bovine serum (FBS) in a CO₂ incubator at 37° C. The generation of the ROS in the SH-SY5Y cells is measured as accumulated oxidized carboxy-H₂DCFDA within a certain period of time as described by Ouyang and Shen (Neurochem. 2006; 97:234-244). Briefly, the cells (when they reach approximately 80% confluence) are seeded on six-well plates are washed twice with serum-free DMEM and thereafter incubated for 4 h in DMEM without serum in the presence of 100 nM xanthophyll derivatives. After washing with DMEM without serum, the cells are loaded with carboxy-H₂DCFDA for 30 min, prior to exposure to 50 μM 6-OHDA for 45 min. Followed by treatment with 6-OHDA, the cells are washed once with PBS (+) and PBS (−), respectively, and then collected into vials. The fluorescence of dichlorofluorescein (DCF) in the supernatant is measured by an EPICS Elite Flow Cytometer (Beckman Coulter, Inc., USA). The data are analyzed using the Bonferroni/Dunn multiple comparison procedure.

Measuring cellular oxidative stress is HaCaT cells: HaCaT cells are seeded in 96-well plates (NuncTM black with optical bottom, Thermo Fisher Scientific, Inc., Waltham, Mass.) with a density at 2×10⁴ cells per well and 100 μL medium in each well, and grown overnight before treatments. Stock solutions of xanthophyll derivatives and other test compounds are prepared and diluted to 2× of final treatment concentrations in DMEM medium in 96-well storage plates. HaCaT Cells are treated in triplicates by adding 100 μL of diluted solutions of test compounds (2×) to each well containing 100 μl medium already. Cells are incubated in the presence of test compounds for 3 hrs. At the end of 3 hr incubation, cells are washed once with 200 μL DPBS with Ca⁺⁺ and Mg⁺⁺ (Life Technologies) and then fed with 100 μL DMEM medium each well containing H₂O₂ at 250 μM (as a ROS (reactive oxygen species) inducer, Sigma). After 30 min incubation, cells are washed once with 200 μL DPBS with Ca⁺⁺ and Mg⁺⁺ and CelIROX deep red reagent (2.5 mM stock solution, Life Technologies) is added to the medium to a final concentration of 5 μM. After 1 hr incubation at 37° C., 5% CO₂, Cells are washed for 3 times with DPBS, fluorescence signal is read at Ex. 640 nm and Em. 665 nm in Biotek Synergy H₄ microplate reader (BioTek, Winooski, Vt.). Untreated cells serve as controls (normalized to 1). All the treated samples are normalized to the untreated control. Hydroxytyrosol at 0.005%, or other antioxidant serves as a positive control.

### Ischemia-Reperfusion Injury

An in vivo mouse model of ischemia-reperfusion injury provides data pertaining to long-term effects of ischemia-reperfusion on the myocardium and demonstrates the protective effects of mitochondria-targeted prodrugs of the instant invention. The use of such a model is well-known in the art (See e.g., Shishido, et al. Circulation 2003; 108:2905-2910) with ischemia-reperfusion carried out by occluding the left anterior descending coronary artery (LAD) for 45 minutes, followed by 24 hours of reperfusion. End points measured include infarct size, in addition to isolation of mitochondria from cardiac tissue, and measurement of mitochondrial functional parameters. Mitochondrial-targeted xanthophyll derivatives are administered via bolus IV injection 1 hour prior to the LAD occlusion protocol, at an initial dose of 0.2 mg/kg, which is equivalent to a plasma concentration of 14 1.J.M based on established mouse toxicology models (See e.g., Diehl, et al. J. Appl. Toxicol. 2001; 21:15-23).

### Crystallization and Polymorphism

The compounds of the present invention may have one or more kinds of crystal form or non-crystalline form. As a method for producing different crystal forms, they can be produced by a method to obtain a crystal polymorphism in an active ingredient of a medicine. Examples of the method for producing the crystal forms include a method to dissolve the compound of the present invention in a solvent and to precipitate the compound by adding a poor solvent; a method to precipitate the compound by distill the solvent off; a method to precipitate the compound by lowering temperature and so on, and these methods may be combined. The crystal of the compound of the present invention can be precipitated selectively and easily by adding a desired seed crystal. The noncrystalline form can be produced by using a method to obtain a noncrystalline form in an active ingredient of a medicine. Examples of the method for producing a noncrystalline form include a method to enhance the precipitation speed at the time to produce the crystal form described above, a method to add a crystallization inhibitor such as a polymer at the time to dissolve, a method to melt using a biaxial extruder by adding a melting point depressant and thus controlling physical state.

### Salt and Cocrystal Formation

Salts and co-crystals of the compounds of the invention, include without limitation, pharmaceutically acceptable salts prepared by treating the free base with appropriate acids, such as organic or inorganic acids, including without limitation, malic acid, hydrochloric acid, sulfuric acid, fumaric acid, phosphoric acid, tartaric acid, maleic acid, malonic acid, adipic acid, benzenesulfonic acid, and the like. For example, the process for forming a salt or co-crystal can be carried out in a solvent system in which both reactants (e.g., a xanthophyll derivative free base and the respective acid) are sufficiently soluble. In one method, to achieve crystallization or precipitation, a solvent or solvent mixture in which the resulting salt and co-crystal is only slightly soluble or not soluble at all is used. Alternatively, a solvent in which the desired salt and co-crystal is very soluble can be used, and then an anti-solvent (or a solvent in which the resulting salt is poorly soluble) is added to the solution. Other variants for salt formation or crystallization includes concentrating the salt and co-crystal solution (e.g., by heating, under reduced pressure if necessary, or by slowly evaporating the solvent, for example, at room temperature), or seeding with the addition of seed crystals, or setting up water activity required for hydrate formation.

All references, including publications, PCT and patent applications, and patents, cited herein are hereby incorporated by reference to the same extent as if each reference were individually and specifically indicated to be incorporated by reference and were set forth in its entirety herein.

1. A compound of any one formula selected from any one of (XVI)-(XXX) or a pharmaceutically acceptable salt or co-crystal thereof: 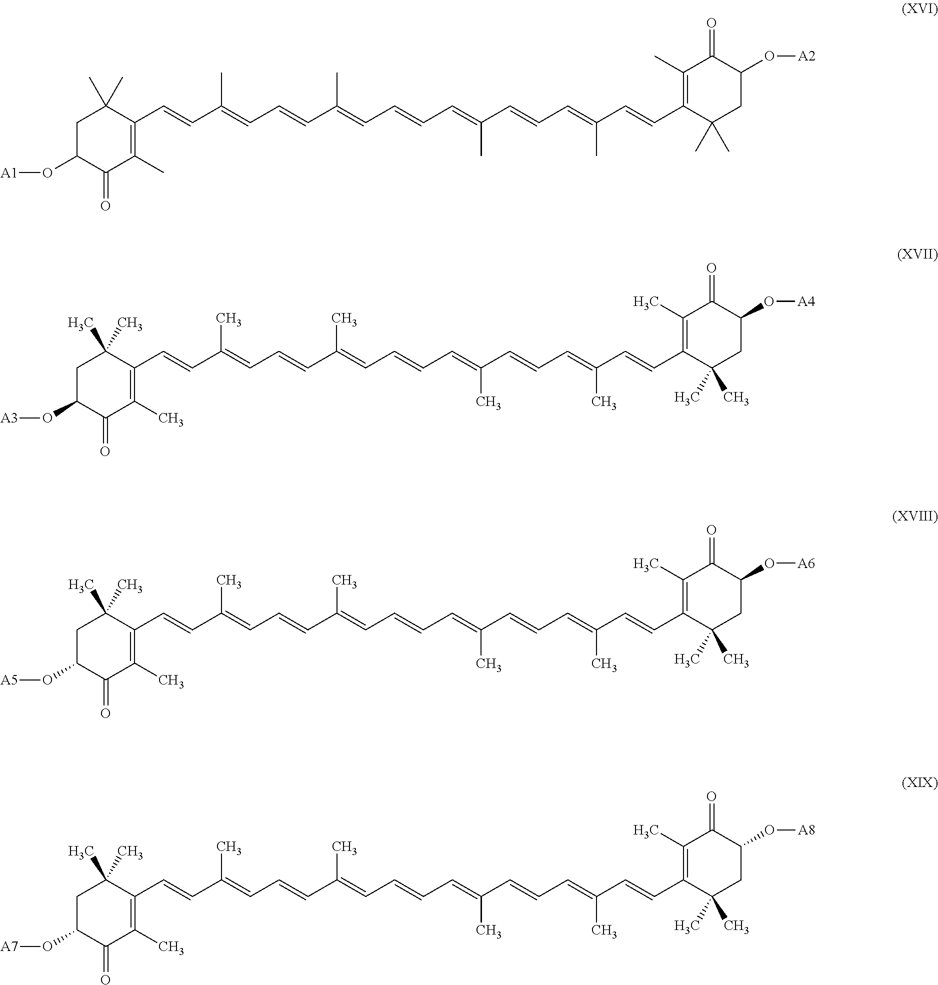 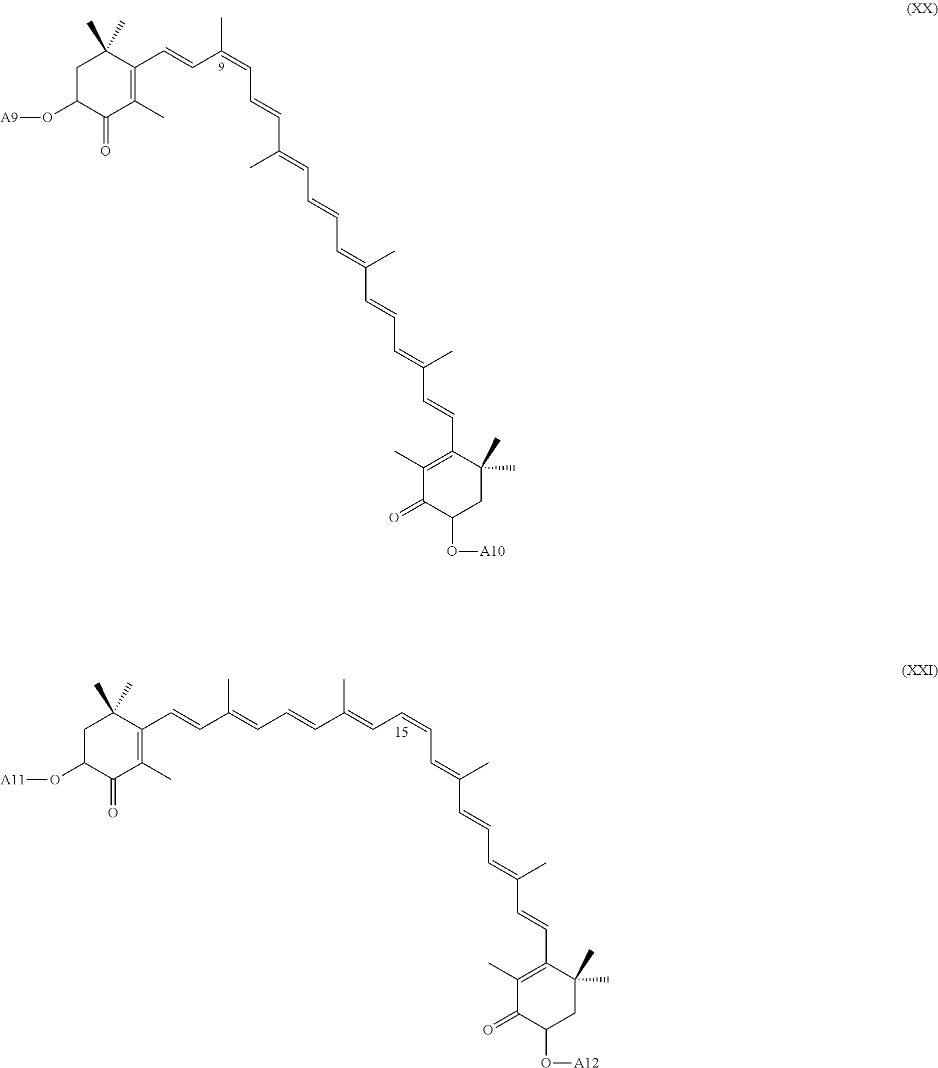 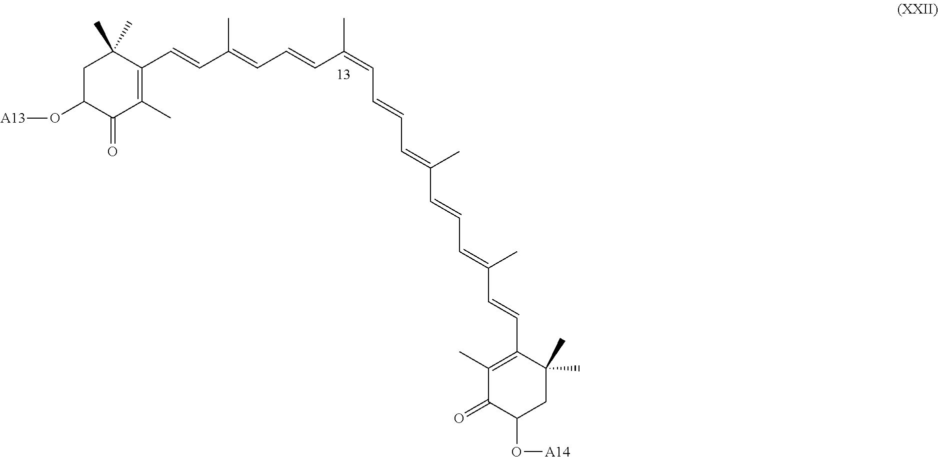 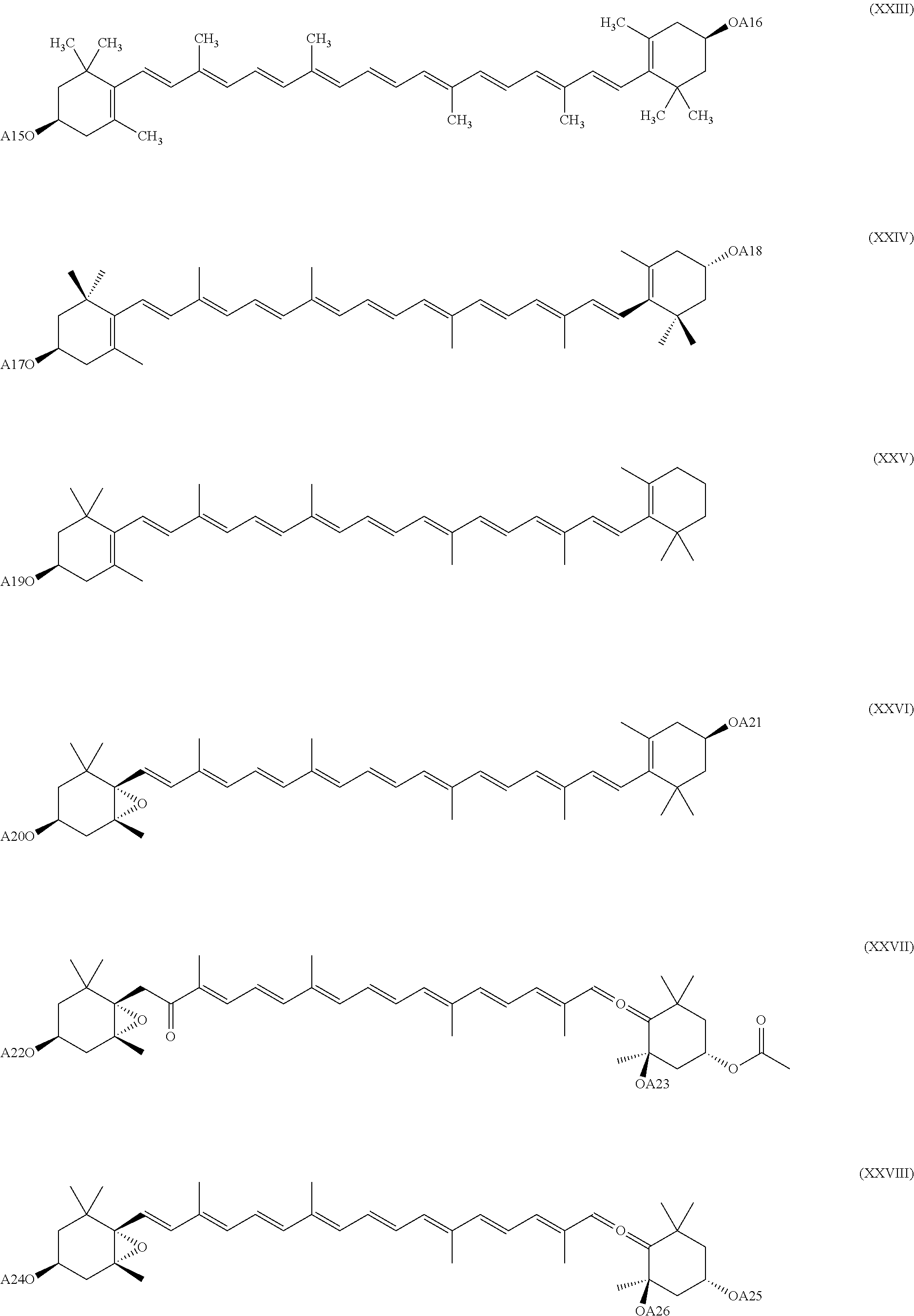 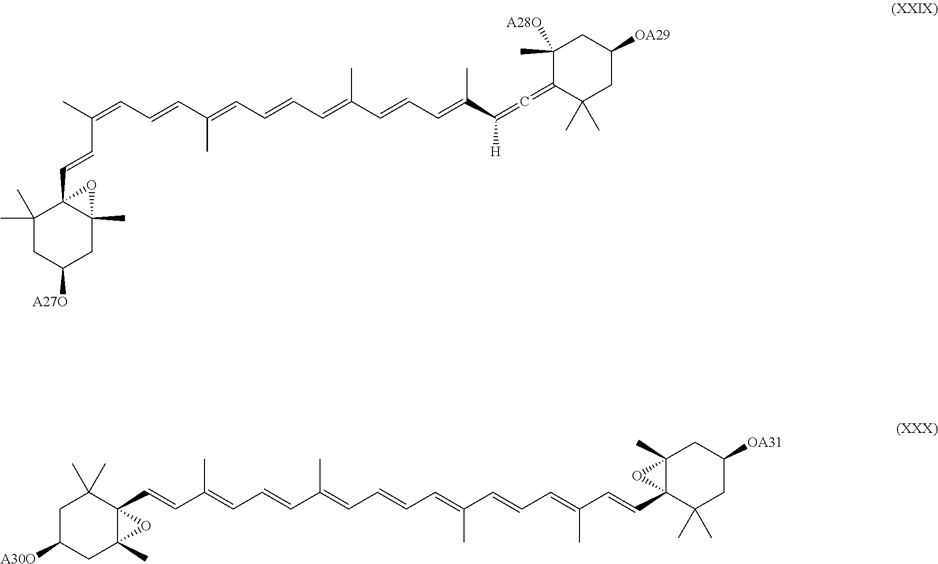
wherein each substituent A1-A31 is independently R1 or —H, with the proviso that at least one substituent on each of said formula is R1; and wherein R1 is selected from: acyl; amide, imide; ester; or —NO₂.

2-58. (canceled)
